(19) United States
(12) Patent Application Publication
(10) Pub. No.: US 2023/0225987 A1
(43) Pub. Date: Jul. 20, 2023

(54) TOPICAL PHARMACEUTICAL COMPOSITIONS

(71) Applicant: Dermavant Sciences GmbH, Basel (CH)

(72) Inventors: Piyush JAIN, Durham, NC (US); Robert Michael LEMING, Morganton, NC (US); Glenn TABOLT, Varina, NC (US); Alan Scott COLBORN, Durham, NC (US)

(73) Assignee: Dermavant Sciences GmbH, Basel (CH)

(21) Appl. No.: 18/002,511

(22) PCT Filed: Jun. 24, 2021
(86) PCT No.: PCT/US2021/038794

§ 371 Date: Dec. 20, 2022

Related U.S. Application Data
(60) Provisional application No. 63/043,340, filed on Jun. 24, 2020.
(60) Provisional application No. 63/043,360, filed on Jun. 24, 2020.

(51) Int. Cl. A61K 31/05; A61K 47/06; A61K 9/107; A61K 9/00

(52) U.S. Cl. CPC A61K 31/05; A61K 47/06; A61K 9/1075; A61K 9/0014

(57) ABSTRACT

The present invention relates to topical pharmaceutical emulsion compositions comprising a therapeutically effective amount of 3,5-Dihydroxy-4-isopropyl-trans-stilbene or a pharmaceutically acceptable salt thereof, an oil phase, a water phase, a surfactant, and an antioxidant, and wherein the emulsion composition is homogeneous and/or the active is solubilized in the oil phase. The invention also relates to methods of treating a dermatological condition or disorder in a patient by administering the present compositions to the skin of the patient.

### CROSS-REFERENCE TO RELATED APPLICATIONS

This application claims priority to and benefit of U.S. Provisional Application No. 63/043,340 filed Jun. 24, 2020 and of U.S. Provisional Application No. 63/043,360 filed Jun. 24, 2020, the entire contents of which are hereby incorporated by reference.

### FIELD OF THE INVENTION

The present invention relates to topical pharmaceutical compositions.

### BACKGROUND OF THE INVENTION

A challenge for the formulation chemist is to prepare a physically stable topical pharmaceutical composition where the active ingredient is also found to be chemically stable.

Such pharmaceutical compositions should:
(i) not irritate the skin,
(ii) be specifically adapted to deliver the active ingredient onto or into the skin so as to treat a particular dermatological condition or disorder,
(iii) be cosmetically elegant to ensure that the patient complies with the prescribed treatment regimen,
(iv) provide penetration of the active ingredient to the appropriate layers of the skin and engage the desired target, and
(v) minimize systemic exposure while achieving local dermal/epidermal delivery.

One active ingredient of interest to be formulated in a physically and chemically stable topical composition is 3,5-Dihydroxy-4-isopropyl-trans-stilbene which has the following formula:

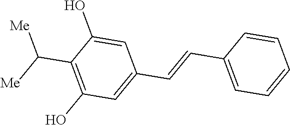 [SMILES: CC(C)c1c(O)cc(/C=C/c2ccccc2)cc1O]

This compound is also known as 5-[(E)-2-phenylethenyl]-2-(propan yl)benzene-1,3-diol or 2-(1-Methyethyl)-5-[(1E)-2-phenylethenyl]-1,3-benzenediol.

3,5-Dihydroxy-4-isopropyl-trans-stilbene is believed to have been originally disclosed by Paul et al., Journal of Chemical Ecology 1981 7(3): 589-597 as an antibiotic. Li et al, Applied and Environmental Microbiology 1995 61 (12): 4329-4333 also isolated the compound, but from a different bacterial strain and further demonstrated its fungicidal activity. The fungicidal activity of the compound was also described in WO 1995/003695 (Agro-Biotech Corporation). The compound is further described in WO 2001/042231 (Welichem Biotech Inc.) and in U.S. Pat. No. 7,868,047 and as being suitable for the treatment of various key dermatological conditions including psoriasis and inflammation. Example 3 of the U.S. Pat. No. 7,868,047 describes a cream formulation with an active ingredient being made in Galax Base. Applicants have been unable to ascertain any compendial notations or availability of a commercial cream base called “Galax” and therefore its composition remains unknown.

3,5-Dihydroxy-4-isopropyl-trans-stilbene is known to be sensitive to oxidation and photo degradation (see e.g., Gao et al., Journal of Polymer Research 2011 18: 1501-1508). Accordingly, there remains a need in the art for a topical composition comprising 3,5-Dihydroxy-4-isopropyl-trans-stilbene that is both chemically and physically stable, which delivers the active ingredient to the desired site of action in the epidermis and/or dermis, and which does not irritate the skin in use.

U.S. application Ser. No. 15/807,682 (Publication No. 2018/0064656) filed Nov. 9, 2017, is herein incorporated by reference.

### BRIEF DESCRIPTION OF THE DRAWINGS

FIG. 1A illustrates the appearance of a non-uniform emulsion that was characteristic of Formulation 12. FIG. 1B illustrates the appearance of a non-uniform emulsion that was characteristic of Formulation 12 compared with the physically stable formulation characterized by Formulation 17, 21-24.

FIG. 2 illustrates the amount of 3,5-Dihydroxy-4-isopropyl-trans-stilbene delivered into the epidermis and dermis from Formulations 1, 12, 17 and 21-24.

FIG. 3 illustrates the amount of 3,5-Dihydroxy-4-isopropyl-trans-stilbene delivered into the receiving fluid over 15 hours from Formulations 1, 12, 17, and 21-24.

FIG. 4 illustrates the amount of 3,5-Dihydroxy-4-isopropyl-trans-stilbene delivered into the dermis at 3, 6, 9, 12 and 15 hours from Formulations 12 and 21.

FIG. 5 illustrates the amount of 3,5-Dihydroxy-4-isopropyl-trans-stilbene delivered into the receiving fluid over 72 hours from Formulations 12 and 21.

FIG. 6 illustrated the percent change of mRNA Cyp1A1 in human ex vivo skin after Th17 stimulation for Formulations 12, 17, 21 and 22.

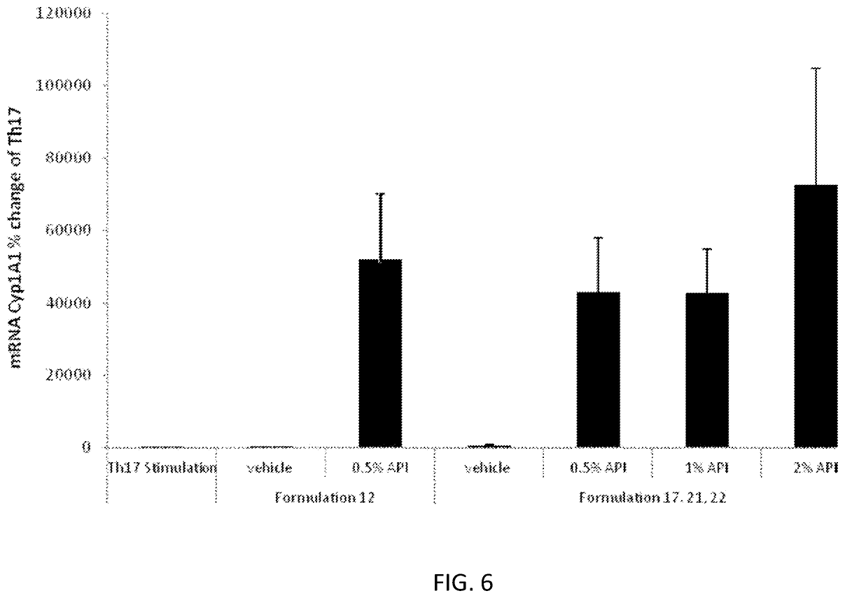

FIG. 7 illustrates the amount of 3,5-Dihydroxy-4-isopropyl-trans-stilbene delivered into skin of Gottingen mini pigs after 7 days of repeat dosing.

FIG. 8 illustrates the amount of 3,5-Dihydroxy-4-isopropyl-trans-stilbene in the plasma of Gottingen mini pigs after 7 days of repeat dosing. Formulation in FIGS. 7 and 8 correspond to Formulations 12 and 22.

### SUMMARY OF THE INVENTION

In one embodiment, the present invention provides for a topical pharmaceutical emulsion composition comprising the active ingredient 3,5-Dihydroxy-4-isopropyl-trans-stilbene or a pharmaceutically acceptable salt thereof, an oil phase, a water phase, a surfactant, and an antioxidant, and wherein the emulsion composition is homogeneous.

In one embodiment, the present invention provides for a topical pharmaceutical emulsion composition comprising the active ingredient 3,5-Dihydroxy-4-isopropyl-trans-stilbene or a pharmaceutically acceptable salt thereof, an oil phase, a water phase, a surfactant, and an antioxidant, and wherein the emulsion composition is uniform.

The present disclosure provides for formulations which effectively solubilize 3,5-Dihydroxy-4-isopropyl-trans-stilbene, or a pharmaceutically acceptable salt thereof, resulting in a homogeneous and uniform emulsion. Homogeneity and uniformity can be determined using microscopy, hot stage optical microscopy, XRD, IR microscopy and fluorescent microscopy and by analyzing transmission profile, droplet size, droplet shape, viscosity and/or stability index.

In another embodiment, the active is solubilized in the oil phase of the emulsion composition. In another embodiment, the oil phase contains mineral oil and/or petrolatum. In another embodiment, if the oil phase contains mineral oil or petrolatum, then a second oil phase component is present, and if the oil phase contains both mineral oil and petrolatum than a third oil phase component is present. In one embodiment, the oil phase is substantially free from petrolatum. In another embodiment, the oil phase contains ≤3%, or ≤2%, or ≤1% petrolatum. In another embodiment, if the oil phase contains both mineral oil and petrolatum at least a third oil phase component is present which is an ester or an ester of glycerin, suitably an ester of glycerin such as a medium chain triglyceride. In another embodiment, if the oil phase contains mineral oil then a second oil phase component is present which is an ester or an ester of glycerin, suitably an ester of glycerin such as a medium chain triglyceride.

In one embodiment, the present invention provides for a topical pharmaceutical emulsion composition comprising an effective amount of the active ingredient 3,5-Dihydroxy-4-isopropyl-trans-stilbene or a pharmaceutically acceptable salt thereof, an oil phase, a water phase, a surfactant, and an antioxidant, wherein the emulsion composition is homogeneous and the 3,5-Dihydroxy-4-isopropyl-trans-stilbene or pharmaceutically acceptable salt thereof is solubilized in the oil phase of the emulsion composition, and provided that if the oil phase contains mineral oil or petrolatum, then a second oil phase component is present, and if the oil phase contains both mineral oil and petrolatum then a third oil phase component is present. In one embodiment, the oil phase is substantially free from petrolatum. In another embodiment, the oil phase contains ≤3%, or ≤2%, or ≤1% petrolatum. In another embodiment, if the oil phase contains both mineral oil and petrolatum at least a third oil phase component is present which is an ester and/or and ester of glycerin, suitably an ester of glycerin, such as a medium chain triglyceride. In another embodiment, if the oil phase contains mineral oil then a second oil phase component is present which is an ester and/or an ester of glycerin, suitably an ester of glycerin, such as a medium chain triglyceride.

In one embodiment, the present invention provides for a topical pharmaceutical emulsion composition comprising the active ingredient 3,5-Dihydroxy-4-isopropyl-trans-stilbene or a pharmaceutically acceptable salt thereof, an oil phase, a water phase, a surfactant, and an antioxidant, and wherein the active is solubilized in the oil phase of the emulsion composition. In another embodiment, the emulsion composition is homogeneous.

In another embodiment, the present invention provides for a topical pharmaceutical emulsion composition comprising the active ingredient 3,5-Dihydroxy-4-isopropyl-trans-stilbene or a pharmaceutically acceptable salt thereof, an oil phase, a water phase, a surfactant, and an antioxidant, and wherein the emulsion composition is homogeneous and/or the active is solubilized in the oil phase of the emulsion composition.

In another embodiment, the present invention provides for a topical pharmaceutical emulsion composition comprising the active ingredient 3,5-Dihydroxy isopropyl-trans-stilbene or a pharmaceutically acceptable salt thereof, an oil phase, a water phase, a surfactant, and an antioxidant, and wherein the average the average droplet size of the discontinuous phase is about 0.1 micron to about 35 microns. In another embodiment, the average the average droplet size of the discontinuous phase is about 0.1 micron to about 25 microns. In another embodiment, the average the average droplet size of the discontinuous phase is about 0.1 micron to about 15 microns. In another embodiment, the average droplet size of the discontinuous phase is about 0.1 micron to about 10 microns. In another embodiment, the average droplet size of the discontinuous phase is about 0.1 micron to about 5 microns. In another embodiment, the average droplet size of the discontinuous phase is about or is about 0.1 micron to about 1 micron. In another embodiment, the average droplet size of the discontinuous phase is from about 0.1 to about 0.75 microns. In another embodiment, the average droplet size of the discontinuous phase is about 0.5 microns.

In another embodiment, the present invention provides for a topical pharmaceutical emulsion composition comprising the active ingredient 3,5-Dihydroxy-4-isopropyl-trans-stilbene or a pharmaceutically acceptable salt thereof, an oil phase, a water phase, a surfactant, and an antioxidant, and wherein the average particle size of the oil phase is about 0.1 micron to about 35 microns, and the emulsion composition is homogeneous and/or the active is solubilized in the oil phase of the emulsion composition.

In another embodiment, the present invention provides for a topical pharmaceutical emulsion composition comprising the active ingredient 3,5-Dihydroxy-4-isopropyl-trans-stilbene or a pharmaceutically acceptable salt thereof, an oil phase, a water phase, a surfactant, and an antioxidant, and wherein the average particle size of the oil phase is about 0.1 micron to about 10 microns, and the emulsion composition is homogeneous and/or the active is solubilized in the oil phase of the emulsion composition.

In another embodiment, the invention provides a method of treating a dermatological condition or disorder in a patient in need thereof, the method comprising administering to said patient a topical pharmaceutical emulsion composition comprising the active ingredient 3,5-Dihydroxy-4-isopropyl-trans-stilbene or a pharmaceutically acceptable salt thereof, an oil phase, a water phase, a surfactant, and an antioxidant, and wherein the emulsion composition is homogeneous.

In another embodiment, the invention provides a method of treating a dermatological condition or disorder in a patient in need thereof, the method comprising administering to said patient a topical pharmaceutical emulsion composition comprising the active ingredient 3,5-Dihydroxy-4-isopropyl-trans-stilbene or a pharmaceutically acceptable salt thereof, an oil phase, a water phase, a surfactant, and an antioxidant, and wherein the emulsion composition is uniform.

In another embodiment, the active is solubilized in the oil phase of the emulsion composition. In another embodiment, the oil phase contains mineral oil and/or petrolatum. In another embodiment, if the oil phase contains mineral oil or petrolatum, then a second oil phase component is present, and if the oil phase contains both mineral oil and petrolatum than a third oil phase component is present. In one embodiment, the oil phase is substantially free from petrolatum. In another embodiment, the oil phase contains ≤3%, or ≤2%, or ≤1% petrolatum. In another embodiment, if the oil phase contains both mineral oil and petrolatum at least a third oil phase component is present which is an ester or an ester of glycerin, suitably an ester of glycerin such as a medium chain triglyceride. In another embodiment, if the oil phase contains mineral oil then a second oil phase component is present which is an ester or an ester of glycerin, suitably an ester of glycerin such as a medium chain triglyceride.

In another embodiment, the invention provides a method of treating a dermatological condition or disorder in a patient in need thereof, the method comprising administering to said patient a topical pharmaceutical emulsion composition comprising an effective amount of the active ingredient 3,5-Dihydroxy-4-isopropyl-trans-stilbene or a pharmaceutically acceptable salt thereof, an oil phase, a water phase, a surfactant, and an antioxidant, wherein the 3,5-Dihydroxy-4-isopropyl-trans-stilbene or pharmaceutically acceptable salt thereof is solubilized in the oil phase of the emulsion composition, and provided that if the oil phase contains mineral oil or petrolatum, then a second oil phase component is present, and if the oil phase contains both mineral oil and petrolatum than a third oil phase component is present. In one embodiment, the oil phase is substantially free from petrolatum. In another embodiment, the oil phase contains ≤3%, or ≤2%, or ≤1% petrolatum. In another embodiment, if the oil phase contains both mineral oil and petrolatum then at least a third oil phase component is present which comprises an ester and/or an ester of glycerin, suitably an ester of glycerin, such as a medium chain triglyceride. In another embodiment, if the oil phase contains mineral oil then a second oil phase component is present which is an ester and/or an ester of glycerin, suitably an ester of glycerin, such as a medium chain triglyceride. In one embodiment, the dermatological condition or disorder is an inflammatory dermatological condition or disorder. In another embodiment, the inflammatory dermatological condition or disorder is atopic dermatitis and/or psoriasis, and/or acne.

In another embodiment, the invention relates to the use of a topical pharmaceutical emulsion composition comprising an effective amount of the active ingredient 3,5-Dihydroxy-4-isopropyl-trans-stilbene or a pharmaceutically acceptable salt thereof, an oil phase, a water phase, a surfactant, and an antioxidant, and wherein the emulsion is homogeneous, for use in the treatment or prophylaxis of a dermatological condition or disorder in a human patient.

In another embodiment, the invention relates to the use of a topical pharmaceutical emulsion composition comprising an effective amount of the active ingredient 3,5-Dihydroxy-4-isopropyl-trans-stilbene or a pharmaceutically acceptable salt thereof, an oil phase, a water phase, a surfactant, and an antioxidant, wherein the 3,5-Dihydroxy-4-isopropyl-trans-stilbene or pharmaceutically acceptable salt thereof is solubilized in the oil phase of the emulsion composition, and provided that if the oil phase comprises mineral oil or petrolatum, then a second oil phase component is present, and if the oil phase contains both mineral oil and petrolatum than a third oil phase component is present. In one embodiment, the oil phase is substantially free from petrolatum. In another embodiment, the oil phase contains ≤3%, or ≤2%, or ≤1% petrolatum. In another embodiment, if the oil phase contains both mineral oil and petrolatum at least a third oil phase component is present which is an ester and/or an ester of glycerin, suitably an ester of glycerin such as a medium chain triglyceride. In another embodiment, if the oil phase contains mineral oil then a second oil phase component is present which is an ester and/or an ester of glycerin, suitably an ester of glycerin, such as a medium chain triglyceride.

In another embodiment, the invention relates to a topical pharmaceutical emulsion composition comprising an effective amount of the active ingredient 3,5-Dihydroxy-4-isopropyl-trans-stilbene or a pharmaceutically acceptable salt thereof, an oil phase, a water phase, a surfactant, and an antioxidant, wherein the 3,5-Dihydroxy-4-isopropyl-trans-stilbene or pharmaceutically acceptable salt thereof is solubilized in the oil phase of the emulsion composition, and provided that if the oil phase contains mineral oil or petrolatum, then a second oil phase component is present, and if the oil phase contains both mineral oil and petrolatum than a third oil phase component is present for use in the treatment or prophylaxis of a dermatological condition or disorder in a human patient. In one embodiment, the oil phase is substantially free from petrolatum. In another embodiment, the oil phase contains ≤3%, or ≤2%, or ≤1% petrolatum. In another embodiment, if the oil phase contains both mineral oil and petrolatum at least a third oil phase component is present which is an ester and/or an ester of glycerin, suitably an ester of glycerin such as a medium chain triglyceride. In another embodiment, if the oil phase contains mineral oil then a second oil phase component is present which is an ester and/or and ester of glycerin, suitably an ester of glycerin, such as a medium chain triglyceride.

In another embodiment, the invention relates to a method of reducing irritancy in a topical pharmaceutical emulsion composition containing the active ingredient 3,5-Dihydroxy-4-isopropyl-trans-stilbene or a pharmaceutically acceptable salt thereof in use in a patient in need thereof, the method comprising administering to the patient a pharmaceutical emulsion composition comprising an oil phase, a water phase, a surfactant, and an antioxidant, wherein the 3,5-Dihydroxy-4-isopropyl-trans-stilbene or pharmaceutically acceptable salt thereof is solubilized in the oil phase of the emulsion composition, and provided that if the oil phase contains mineral oil or petrolatum, then a second oil phase component is present, and if the oil phase contains both mineral oil and petrolatum than a third oil phase component is present. In one embodiment, the oil phase is substantially free from petrolatum. In another embodiment the oil phase contains ≤3%, or ≤2%, or ≤1% petrolatum. In another embodiment, if the oil phase contains both mineral oil and petrolatum at least a third oil phase component is present which is an ester and/or an ester of glycerin, suitably an ester of glycerin, such as a medium chain triglyceride. In another embodiment, if the oil phase contains mineral oil then a second oil phase component is present which is an ester and/or an ester of glycerin, suitably an ester of glycerin such as a medium chain triglyceride.

In one embodiment, the emulsion composition of the instant invention is compared to that of Formulation 1 or 12 (with comparable % w/w active).

In another embodiment, the invention relates to a method of improving the residency time of the active ingredient 3,5-Dihydroxy-4-isopropyl-trans-stilbene or a pharmaceutically acceptable salt thereof in the skin of a patient in need thereof, the method comprising administering to said patient a pharmaceutical emulsion composition comprising an effective amount of 3,5-Dihydroxy-4-isopropyl-trans-stilbene or a pharmaceutically acceptable salt thereof, an oil phase, a water phase, a surfactant, and an antioxidant, wherein the emulsion composition is homogeneous.

In another embodiment, the invention relates to a method of improving the residency time of the active ingredient 3,5-Dihydroxy-4-isopropyl-trans-stilbene or a pharmaceutically acceptable salt thereof in the skin of a patient in need thereof, the method comprising administering to said patient a pharmaceutical emulsion composition comprising an effective amount of 3,5-Dihydroxy-4-isopropyl-trans-stilbene or a pharmaceutically acceptable salt thereof, an oil phase, a water phase, a surfactant, and an antioxidant, wherein the 3,5-Dihydroxy-4-isopropyl-trans-stilbene or pharmaceutically acceptable salt thereof is solubilized in the oil phase of the emulsion composition, and provided that if the oil phase contains mineral oil or petrolatum, then a second oil phase component is present, and if the oil phase contains both mineral oil and petrolatum than a third oil phase component is present. In one embodiment, the oil phase is substantially free from petrolatum. In another embodiment, the oil phase contains ≤3%, or ≤2%, or ≤1% petrolatum. In another embodiment, if the oil phase contains both mineral oil and petrolatum at least a third oil phase component is present which is an ester and/or an ester of glycerin, suitably an ester of glycerin such as a medium chain triglyceride. In another embodiment, if the oil phase contains mineral oil then a second oil phase component is present which is which is an ester and/or an ester of glycerin, suitably an ester of glycerin such as a medium chain triglyceride. In one embodiment, the emulsion composition of the instant invention is compared to that of the Formulation 1 or 12 (with comparable % w/w of active ingredient).

In another embodiment, the invention relates to a method of improving the residency time of the active ingredient 3,5-Dihydroxy-4-isopropyl-trans-stilbene or a pharmaceutically acceptable salt thereof in the skin of a patient in need thereof, the method comprising administering to said patient a pharmaceutical emulsion composition comprising an effective amount of 3,5-Dihydroxy-4-isopropyl-trans-stilbene or a pharmaceutically acceptable salt thereof, an oil phase, a water phase, a surfactant, and an antioxidant, and wherein the emulsion is homogeneous and/or the active is solubilized in the oil phase. In another embodiment, the average droplet size of the discontinuous phase is about 0.1 micron to about 35 microns. In another embodiment, the average droplet size of the discontinuous phase is about 0.1 micron to about 5 microns. In another embodiment, the average droplet size of the discontinuous phase is about 0.1 micron to about 1 micron.

In another embodiment, the invention relates to a method of reducing side effects in a patient who is administered a composition containing 3,5-Dihydroxy-4-isopropyl-trans-stilbene or a pharmaceutically acceptable salt thereof, the method comprising administering to the patient a pharmaceutical emulsion composition comprising an oil phase, a water phase, a surfactant, and an antioxidant, and wherein the emulsion is homogeneous and/or the active is solubilized in the oil phase. In another embodiment, the average droplet size of the discontinuous phase is about 0.1 micron to about 35 microns. In another embodiment, the average droplet size of the discontinuous phase is about 0.1 micron to about 5 microns. In another embodiment, the average droplet size of the discontinuous phase is about 0.1 micron to about 1 micron.

In another embodiment, the invention relates to a method of reducing side effects in a patient who is administered a composition containing 3,5-Dihydroxy-4-isopropyl-trans-stilbene or a pharmaceutically acceptable salt thereof, the method comprising administering to the patient a pharmaceutical emulsion composition comprising an oil phase, a water phase, a surfactant, and an antioxidant, and wherein the 3,5-Dihydroxy-4-isopropyl-trans-stilbene or pharmaceutically acceptable salt thereof is solubilized in the oil phase of the emulsion composition, and provided that if the oil phase contains mineral oil or petrolatum, then a second oil phase component is present, and if the oil phase contains both mineral oil and petrolatum than a third oil phase component is present. In one embodiment, the oil phase is substantially free from petrolatum. In another embodiment, the oil phase contains ≤3%, or ≤2%, or ≤1% petrolatum. In another embodiment, if the oil phase contains both mineral oil and petrolatum at least a third oil phase component is present which is an ester and/or and ester of glycerin, suitably an ester of glycerin such as a medium chain triglyceride. In another embodiment, if the oil phase contains mineral oil then a second oil phase component is present which is an ester and/or and ester of glycerin, suitably an ester of glycerin such as a medium chain triglyceride. In one embodiment, the emulsion composition of the instant invention is compared to that of Formulation 1 or 12, or a similar formulation with equivalent active ingredient present.

In one embodiment, the present invention provides for a topical pharmaceutical emulsion composition comprising an effective amount of 3,5-Dihydroxy-4-isopropyl-trans-stilbene or a pharmaceutically acceptable salt thereof, an oil phase, a water phase, a surfactant, and an antioxidant, wherein the 3, 5-Dihydroxy-4-isopropyl-trans-stilbene or pharmaceutically acceptable salt thereof is solubilized in the oil phase of the emulsion composition. In one embodiment, the oil phase contains mineral oil and/or petrolatum. In another embodiment, if the oil phase contains mineral oil, then a second oil phase component other than petrolatum is present in the composition. In another embodiment, if the oil phase contains petrolatum, then a second oil phase component other than mineral oil is present in the composition. In any of these embodiments it is preferable that the resulting emulsion does not contain non-uniform areas or waxy regions upon storage.

In one embodiment, the present invention provides for a topical pharmaceutical emulsion composition comprising an effective amount of 3,5-Dihydroxy isopropyl-trans-stilbene or a pharmaceutically acceptable salt thereof, an oil phase, a water phase, a surfactant, and an antioxidant, wherein the 3,5-Dihydroxy-4-isopropyl-trans-stilbene or pharmaceutically acceptable salt thereof is solubilized in the oil phase of the emulsion composition, and wherein if the oil phase contains mineral oil, then a second oil phase component other than petrolatum is present in the composition.

In an embodiment, the invention provides for a topical pharmaceutical emulsion composition comprising 3,5-Dihydroxy-4-isopropyl-trans-stilbene or a pharmaceutically acceptable salt thereof, an oil phase, a water phase, a surfactant, and an antioxidant, wherein the 3,5-Dihydroxy-4-isopropyl-trans-stilbene or pharmaceutically acceptable salt thereof is solubilized in the oil phase of the emulsion composition, and wherein if the oil phase contains mineral oil then a second oil phase component other than petrolatum is present in the composition, or if the oil phase contains both mineral oil and petrolatum at least a third oil phase component is present. In another embodiment, the oil phase is substantially free from mineral oil and/or petrolatum.

In one embodiment, the present invention provides for a topical pharmaceutical O/W emulsion composition comprising an effective amount of 3,5-Dihydroxy-4-isopropyl-trans-stilbene or a pharmaceutically acceptable salt thereof, an oil phase, a water phase, a surfactant, and an antioxidant, wherein the 3,5-Dihydroxy-4-isopropyl-trans-stilbene or pharmaceutically acceptable salt thereof is solubilized in the oil phase of the emulsion composition, and the composition is formulated as a cream or lotion. In another embodiment, the cream or lotion is homogeneous and uniform in appearance.

In certain embodiments, the topical pharmaceutical oil-in-water emulsion composition comprises: about 0.1% to about 5% by weight of 3,5-dihydroxy-4-isopropyl-trans-stilbene or a pharmaceutically acceptable salt thereof, an oil phase in an amount of about 5% to about 35% by weight, based on the total weight of the composition and is selected from the group consisting of cocoyl caprylocaprate, isostearyl isostearate, octyldodecanol, jajoba oil, sesame oil, walnut oil, isopropyl palmitate, isopropyl myristate, safflower oil, mineral oil, olive oil, almond oil, and combinations thereof, a water phase in an amount of about 25% to about 85% by weight, based on the total weight of the composition, a surfactant in an amount of about 0.1% to about 20% by weight, based on the total weight of the composition, and an antioxidant.

In certain embodiments, the topical pharmaceutical oil-in-water emulsion composition comprises: about 0.1% to about 5% by weight of 3,5-dihydroxy-4-isopropyl-trans-stilbene or a pharmaceutically acceptable salt thereof, mineral oil in an amount of about 5% to about 15% by weight, based on the total weight of the composition, a co-solvent in an amount of about 1% to about 30% by weight, based on the total weight of the composition, a water phase in an amount of 25% to about 85% by weight, based on the total weight of the composition, a surfactant in an amount of about 0.1% to about 20% by weight, based on the total weight of the composition, comprising a mixture of polysorbate 80, nonionic emulsifying wax (Polawax NF), Steareth 2, and Steareth 20, and an antioxidant.

In certain embodiments, the topical pharmaceutical oil-in-water emulsion composition comprises: about 0.1% to about 5% by weight of 3,5-dihydroxy-4-isopropyl-trans-stilbene or a pharmaceutically acceptable salt thereof, an oil phase in an amount of about 5% to about 45% by weight, based on the total weight of the composition, a water phase in an amount of about 25% to about 85% by weight, based on the total weight of the composition, a surfactant in an amount of about 1% to about 20% by weight, based on the total weight of the composition and is selected from the group consisting of oleoyl polyoxyl-6 glycerides, PEG32 Stearate, PEG 75, PEG 20 cetostearyl ether, ceteareth-12, ceteareth-15, ethylene glycol palmitostearate, cetostearyl alcohol, glycerol monostearate, stearyl alcohol, cetyl alcohol, steareth-2, steareth-20, steareth-21, and combinations thereof, and an antioxidant.

In certain embodiments, the topical pharmaceutical oil-in-water emulsion composition comprises: about 0.1% to about 5% by weight of 3,5-dihydroxy-4-isopropyl-trans-stilbene or a pharmaceutically acceptable salt thereof, an oil phase in an amount of about 5% to about 45% by weight, based on the total weight of the composition, a water phase in an amount of about 25% to about 85% by weight, based on the total weight of the composition, a surfactant in an amount of about 0.1% to about 20% by weight, based on the total weight of the composition, an antioxidant, and a co-solvent in an amount of greater than 30% by weight, based on the total weight of the composition.

### DETAILED DESCRIPTION OF THE INVENTION

In addition to creating a physically and chemically stable pharmaceutical formulation, the present invention also provides for a pharmaceutical formulation which is non-irritating to the skin upon application and use, or is one which is less irritating than any previous formulations used in the development of the active ingredient to date. Another aspect of the invention is a formulation that not only has superior skin penetration and target engagement of the appropriate receptors, but also has significant non-systemic exposure of the active ingredient to the patient upon application and use.

The active ingredient 3,5-Dihydroxy-4-isopropyl-trans-stilbene has caused a number of issues with respect to formulating the compound in a topical formulation. Many formulations exhibited “waxy” regions, seen only in the presence of the active ingredient, and which further were dependent upon the concentration of the active ingredient in the formulation. Waxy regions may also be referred to herein as non-uniform emulsions. In one embodiment, the uniform emulsion includes none, or less than 1% of the active ingredient in crystalline form. As consistency and manufacturability are necessary for commercial batches, a final product with container/tube content homogeneity under higher temperatures and storage is needed.

Various approaches to correct these problems have been investigated. One approach is disclosed in a patent application to GlaxoSmithKline Intellectual Property Development Limited, Sonti et al., PCT/IB2016/052955 and U.S. application Ser. No. 15/158,858 both filed May 19, 2016, whose disclosures are incorporated by reference herein. In these applications, one approach was taken to decrease the amount of mineral oil and/or petrolatum present, in the formulation (if at all present to start with), and to use as an oil phase component, suitably an ester and/or an ester of glycerin, such as a medium chain triglyceride which would assist in solubilizing the active ingredient. This approach produced a uniform and homogeneous emulsion.

However, it is also possible to eliminate the waxy regions, without affecting penetration and release profiles by other modifications to the formulation as further described herein.

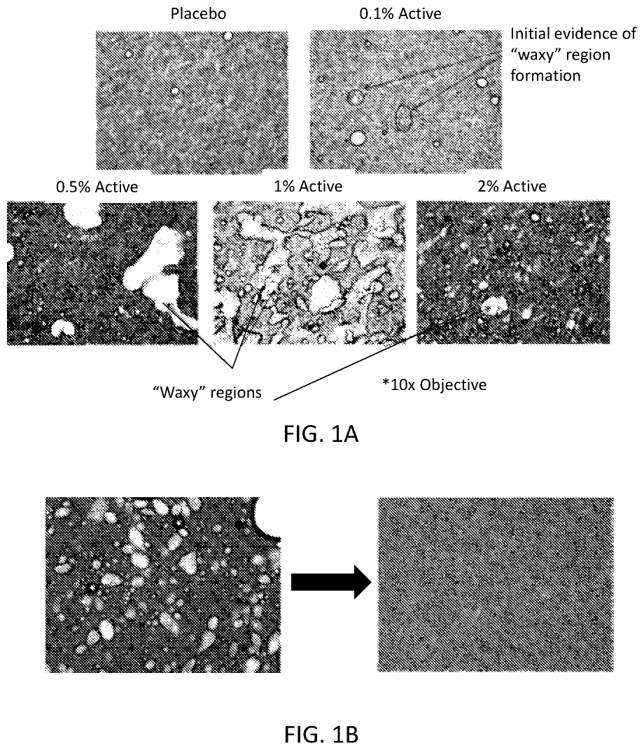

The active ingredient was believed to cause the emulsion destabilization by perhaps interacting at the surfactant interface (liquid crystals). As can be seen in the formulations shown in FIG. 1A the presence of the active ingredient appears to be altering the system in some manner. The non-uniform emulsions illustrated in FIG. 1A 1 were determined to be dependent on the concentration of the active ingredient. Thus, one approach to correcting the waxy regions is to optimize the surfactant system rather than to change the oil phase components as described in Sonti et al. supra above.

In embodiments described herein, the active ingredient, 3,5-Dihydroxy-4-isopropyl-trans-stilbene or a pharmaceutically acceptable salt thereof, is not solubilized in the oil phase, but rather is solubilized in the water phase alone. In embodiments described herein, the oil phase is solely mineral oil and the active ingredient is solubilized in a co-solvent, which is present in an amount of about 1% to about 30%. The co-solvent may be propylene glycol, Transcutol P or a combination thereof.

In embodiments described herein, the active ingredient, 3,5-Dihydroxy isopropyl-trans-stilbene or a pharmaceutically acceptable salt thereof, is solubilized in the water phase alone or in both the water phase and oil phase. In embodiments described herein, co-solvents are included in amounts greater than 30%. High concentrations of co-solvents in the emulsions described herein improve the solubility of the active ingredient in the water phase alone or in both the water phase and oil phase.

In an embodiment, the invention provides for a topical pharmaceutical emulsion composition comprising the active ingredient 3,5-Dihydroxy-4-isopropyl-trans-stilbene or a pharmaceutically acceptable salt thereof, an oil phase, a water phase, a surfactant, and an antioxidant, and wherein the emulsion composition is homogeneous.

In an embodiment, the invention provides for a topical pharmaceutical emulsion composition comprising the active ingredient 3,5-Dihydroxy-4-isopropyl-trans-stilbene or a pharmaceutically acceptable salt thereof, an oil phase, a water phase, a surfactant, and an antioxidant, and wherein the emulsion composition is homogeneous or uniform, upon ICH storage conditions.

In an embodiment, the invention provides for a topical pharmaceutical emulsion composition comprising the active ingredient 3,5-Dihydroxy-4-isopropyl-trans-stilbene or a pharmaceutically acceptable salt thereof, an oil phase, a water phase, a surfactant, and an antioxidant, and wherein the emulsion composition is homogeneous. In one embodiment, the 3,5-Dihydroxy-4-isopropyl-stilbene or pharmaceutically acceptable salt thereof is solubilized in the oil phase of the emulsion composition. In another embodiment, if the oil phase contains mineral oil or petrolatum, then a second oil phase component is present, and if the oil phase contains both mineral oil and petrolatum than a third oil phase component is present. In one embodiment, the oil phase is substantially free from petrolatum. In another embodiment, the oil phase contains ≤3%, or ≤2%, or ≤1% petrolatum. In another embodiment, if the oil phase contains both mineral oil and petrolatum then at least a third oil phase component is present which is an ester and/or an ester of glycerin, suitably an ester of glycerin, such as a medium chain triglyceride. In another embodiment, if the oil phase contains mineral oil, then a second oil phase component is present which is an ester and/or an ester of glycerin, suitably an ester of glycerin, such as a medium chain triglyceride. The second oil phase component and the third oil phase component are used as co-solvents for the active ingredient in the oil phase of the emulsion composition. That is, the second and third oil phase components act as oil miscible co-solvents.

In one embodiment, the amount of the active ingredient solubilized in the oil phase of the emulsion composition is present in an amount of ≥50% w/w, or ≥60% w/w, or ≥70% w/w, or ≥80% w/w, or ≥90% w/w or ≥95% w/w or ≥98% w/w, based on the percent by weight of the active ingredient. In a preferred embodiment≥95% or ≥98% w/w of the active ingredient is solubilized in the oil phase of the emulsion, producing a homogeneous composition.

In an alternative embodiment, the 3,5-Dihydroxy-4-isopropyl-trans-stilbene or a pharmaceutically acceptable salt thereof is solubilized in the water phase of the emulsion composition. Since 3,5-Dihydroxy-4-isopropyl-trans-stilbene is not soluble in water, a water miscible organic solvent (i.e., a water miscible co-solvent may be used to solubilize the active ingredient in the water phase of the emulsion). Suitably, the active ingredient solubilized in the water phase of the emulsion composition is present in an amount ≥10% w/w, or ≥20% w/w, or ≥30% w/w, or ≥40% w/w, or ≥50% w/w, or ≥60% w/w, or ≥70% w/w, or ≥80% w/w, or ≥90% w/w or ≥95% w/w, based on the percent by weight of the active ingredient. In one embodiment, a water miscible co-solvent is selected from Propylene glycol, Transcutol, or PEG 400, or combinations or mixtures thereof.

When used herein, the term “D90” refers to the oil droplet size diameter of which 90% of the droplets are less than a particular size. Alternatively, the term “D90” is defined as the size in microns below which 90 percent of the oil droplets reside on a volume basis. In an embodiment, the D90 of the average droplet size of the discontinuous phase in the composition is about 0.1 micron to about 35 microns. In another embodiment, the D90 of the average droplet size of the discontinuous phase in the composition is between about 0.1 and about 30 microns. In another embodiment, the D90 of the average droplet size of the discontinuous phase in the composition is between about 0.1 and about 25 microns. In another embodiment, the D90 of the average droplet size of the discontinuous phase in the composition is between about 0.1 and about 20 microns. In another embodiment, the D90 of the average droplet size of the discontinuous phase in the composition is between about 0.1 and about 15 microns. In another embodiment, the D90 of the average droplet size of the discontinuous phase in the composition is between about 0.1 and about 10 microns. In another embodiment, the D90 of the average droplet size of the discontinuous phase in the composition is between about 0.1 and 5 microns. In another embodiment, the D90 of the average droplet size of the discontinuous phase in the composition is between about 0.5 and 1 micron. In an embodiment, the D90 of the average droplet size of the discontinuous phase in the composition is about 0.5 micron to about 5 microns. In an embodiment, the D90 of the average droplet size of the discontinuous phase in the composition is about 1 micron to about 5 microns. In an embodiment, the D90 of the average droplet size of the discontinuous phase in the composition is about 1 micron to about 10 microns. In an embodiment, the D90 of the average droplet size of the discontinuous phase in the composition is about 1 micron to about 15 microns. In an embodiment, the D90 of the average droplet size of the discontinuous phase in the composition is about 1 micron to about 20 microns. In an embodiment, the D90 of the average droplet size of the discontinuous phase in the composition is about 1 micron to about 25 microns. In an embodiment, the D90 of the average droplet size of the discontinuous phase in the composition is about 1 micron to about 30 microns. In an embodiment, the D90 of the average droplet size of the discontinuous phase in the composition is about 1 micron to about 35 microns. In an embodiment, the D90 of the average droplet size of the discontinuous phase in the composition is about 5 micron to about 10 microns. In an embodiment, the D90 of the average droplet size of the discontinuous phase in the composition is about 5 micron to about 15 microns. In an embodiment, the D90 of the average droplet size of the discontinuous phase in the composition is about 5 micron to about 20 microns. In an embodiment, the D90 of the average droplet size of the discontinuous phase in the composition is about 15 micron to about 25 microns. In an embodiment, the D90 of the average droplet size of the discontinuous phase in the composition is about 15 micron to about 30 microns. In an embodiment, the D90 of the average droplet size of the discontinuous phase in the composition is about 15 micron to about 35 microns. In an embodiment, the D90 of the average droplet size of the discontinuous phase in the composition is about 10 micron to about 15 microns. In an embodiment, the D90 of the average droplet size of the discontinuous phase in the composition is about 10 micron to about 20 microns. In an embodiment, the D90 of the average droplet size of the discontinuous phase in the composition is about 10 micron to about 25 microns. In an embodiment, the D90 of the average droplet size of the discontinuous phase in the composition is about 10 micron to about 30 microns. In an embodiment, the D90 of the average droplet size of the discontinuous phase in the composition is about 10 micron to about 35 microns. In an embodiment, the D90 of the average droplet size of the discontinuous phase in the composition is about 15 micron to about 20 microns. In an embodiment, the D90 of the average droplet size of the discontinuous phase in the composition is about 15 micron to about 25 microns. In an embodiment, the D90 of the average droplet size of the discontinuous phase in the composition is about 15 micron to about 30 microns. In another embodiment, the D90 of the average droplet size of the discontinuous phase in the composition is about 15 to about 35 microns. In another embodiment, the D90 of the average droplet size of the discontinuous phase in the composition is about 20 to about 35 microns. In another embodiment, the D90 of the average droplet size of the discontinuous phase in the composition is about 25 to about 35 microns. In another embodiment, the D90 of the average droplet size of the discontinuous phase in the composition is about 30 to about 35 microns.

When used herein, the term “D50” refers to the median or 50^{th} percentile of which the droplets are less than a particular size. Alternatively, the term “D50” is defined as the size in microns below which 50 percent of the oil droplets reside on a volume basis. In an embodiment, the D50 of the average droplet size of the discontinuous phase in the composition is about 0.1 micron to about 35 microns. In another embodiment, the D50 of the average droplet size of the discontinuous phase in the composition is between about 0.1 and about 30 microns. In another embodiment, the D50 of the average droplet size of the discontinuous phase in the composition is between about 0.1 and about 25 microns. In another embodiment, the D50 of the average droplet size of the discontinuous phase in the composition is between about 0.1 and about 20 microns. In another embodiment, the D50 of the average droplet size of the discontinuous phase in the composition is between about 0.1 and about 15 microns. In another embodiment, the D50 of the average droplet size of the discontinuous phase in the composition is between about 0.1 and about 10 microns. In an embodiment, the D50 of the average droplet size of the discontinuous phase in the composition is about 0.1 micron to about 5 microns. In another embodiment, the average droplet size of the discontinuous phase according to the present invention have a D50 of about 0.1 micron to about 1 micron. In another embodiment, the D50 of the average droplet size of the discontinuous phase in the composition is between about 0.5 and 1 micron. In an embodiment, the D50 of the average droplet size of the discontinuous phase in the composition is about 0.5 micron to about 5 microns. In an embodiment, the D50 of the average droplet size of the discontinuous phase in the composition is about 1 micron to about 5 microns. In an embodiment, the D50 of the average droplet size of the discontinuous phase in the composition is about 1 micron to about 10 microns. In an embodiment, the D50 of the average droplet size of the discontinuous phase in the composition is about 1 micron to about 15 microns. In an embodiment, the D50 of the average droplet size of the discontinuous phase in the composition is about 1 micron to about 20 microns. In an embodiment, the D50 of the average droplet size of the discontinuous phase in the composition is about 1 micron to about 25 microns. In an embodiment, the D50 of the average droplet size of the discontinuous phase in the composition is about 1 micron to about 30 microns. In an embodiment, the D50 of the average droplet size of the discontinuous phase in the composition is about 1 micron to about 35 microns. In an embodiment, the D50 of the average droplet size of the discontinuous phase in the composition is about 5 micron to about 10 microns. In an embodiment, the D50 of the average droplet size of the discontinuous phase in the composition is about 5 micron to about 15 microns. In an embodiment, the D50 of the average droplet size of the discontinuous phase in the composition is about 5 micron to about 20 microns. In an embodiment, the D50 of the average droplet size of the discontinuous phase in the composition is about 15 micron to about 25 microns. In an embodiment, the D50 of the average droplet size of the discontinuous phase in the composition is about 15 micron to about 30 microns. In an embodiment, the D50 of the average droplet size of the discontinuous phase in the composition is about 15 micron to about 35 microns. In an embodiment, the D50 of the average droplet size of the discontinuous phase in the composition is about 10 micron to about 15 microns. In an embodiment, the D50 of the average droplet size of the discontinuous phase in the composition is about 10 micron to about 20 microns. In an embodiment, the D50 of the average droplet size of the discontinuous phase in the composition is about 10 micron to about 25 microns. In an embodiment, the D50 of the average droplet size of the discontinuous phase in the composition is about 10 micron to about 30 microns. In an embodiment, the D50 of the average droplet size of the discontinuous phase in the composition is about 10 micron to about 35 microns. In an embodiment, the D50 of the average droplet size of the discontinuous phase in the composition is about 15 micron to about 20 microns. In an embodiment, the D50 of the average droplet size of the discontinuous phase in the composition is about 15 micron to about 25 microns. In an embodiment, the D50 of the average droplet size of the discontinuous phase in the composition is about 15 micron to about 30 microns. In another embodiment, the D50 of the average droplet size of the discontinuous phase in the composition is about 15 to about 35 microns. In another embodiment, the D50 of the average droplet size of the discontinuous phase in the composition is about 20 to about 35 microns. In another embodiment, the D50 of the average droplet size of the discontinuous phase in the composition is about 25 to about 35 microns. In another embodiment, the D50 of the average droplet size of the discontinuous phase in the composition is about 30 to about 35 microns.

When used herein, the term “D10” refers to the oil droplet size diameter of which 10% of the droplets are less than a particular size. Alternatively, the term “D10” is defined as the size in microns below which 10 percent of the oil droplets reside on a volume basis.

Methods for measuring oil droplet size distribution are well known in the art. In one embodiment, oil droplet size diameter distribution oil droplet size in the composition according to the present invention may be measured using a laser diffraction techniques. Suitable laser diffraction apparatus includes, for example, the Sympatec HELOS/QUIXEL, or the Malvern Laser Diffractonamer obtainable from Malvern Instruments, Malvern, UK as well as others.

In one embodiment, the present invention provides for a topical pharmaceutical emulsion composition comprising the active ingredient 3,5-Dihydroxy-4-isopropyl-trans-stilbene or a pharmaceutically acceptable salt thereof, an oil phase, a water phase, a surfactant, and an antioxidant, and wherein the average droplet size of the discontinuous phase is about 0.1 micron to about 35 microns. In another embodiment, the average droplet size of the discontinuous phase is about 0.1 micron to about 25 microns. In another embodiment, the average droplet size of the discontinuous phase is about 0.1 micron to about 15 microns. In another embodiment, the average droplet size of the discontinuous phase is about 0.1 micron to about 10 microns. In another embodiment, the average droplet size of the discontinuous phase is about 0.1 micron to about 5 microns. In another embodiment, the average droplet size of the discontinuous phase is about 0.1 micron to about 1 micron. In another embodiment, the average droplet size of the discontinuous phase is from about 0.05 to about 35 microns. In another embodiment, the average droplet size of the discontinuous phase is from about 0.05 to about 5 micron. In another embodiment, the average droplet size of the discontinuous phase is from about 0.05 to about 1 micron. In another embodiment, the average droplet size of the discontinuous phase is from about 0.1 to about 0.75 microns.

In another embodiment, the D90 of the discontinuous phase is from about 15 to about 35 microns.

In another embodiment, the D90 of the discontinuous phase is from about 20 to about 35 microns.

In another embodiment, the D90 of the discontinuous phase is from about 25 to about 35 microns.

In another embodiment, the D90 of the discontinuous phase is from about 0.05 to about 5 microns.

In another embodiment, the D90 of the discontinuous phase is from about 0.05 to about 1 micron.

In another embodiment, the D90 of the discontinuous phase is from about 0.1 to about 0.75 micron.

In another embodiment, the average droplet size of the discontinuous phase is from about 15 to about 35 microns.

In another embodiment, the average droplet size of the discontinuous phase is from about 20 to about 35 microns.

In another embodiment, the average droplet size of the discontinuous phase is from about 25 to about 35 microns.

In another embodiment, the average droplet size of the discontinuous phase is from about 0.5 to about 1 micron.

In another embodiment, the average droplet size of the discontinuous phase is from about 0.1 to about 5 microns.

In another embodiment, the average droplet size of the discontinuous phase is from about 0.1 to about 1 micron.

In one embodiment, at least 90% of the droplets in the oil phase of the oil-in-water emulsion (e.g., the discontinuous phase) have a droplet size of about 0.1 micron to about 35 microns. In another embodiment, at least 95, 97, 98, or 99% of the droplets are about 0.1 micron to about 35 microns. In one embodiment, at least 90% of the droplets in the oil phase of the oil-in-water emulsion (e.g., the discontinuous phase) have a droplet size of about 15 micron to about 35 microns. In another embodiment, at least 95, 97, 98, or 99% of the droplets are about 15 micron to about 35 microns.

In one embodiment, at least 90% of the droplets in the oil phase of the oil-in-water emulsion (e.g., the discontinuous phase) have a droplet size of about 0.1 micron to 1 micron. In another embodiment, at least 95, 97, 98, or 99% of the droplets are about 0.1 micron to about 1 micron. In one embodiment, at least 90% of the droplets in the oil phase of the oil-in-water emulsion (e.g., the discontinuous phase) have a droplet size of about 1 micron to about 35 microns. In another embodiment, at least 95, 97, 98, or 99% of the droplets are about 1 micron to about 35 microns.

Alternatively, or at least in addition to, at least about 75%, or at least about 85%, or at least about 90% of the droplet size of the discontinuous phase oil in the oil-in-water emulsion have a size of about 0.1 micron to about 35 microns, about 0.1 micron to about 10 microns, about 0.1 micron to about 5 microns, about 0.1 micron to about 1 micron, or about 0.1 micron to about 0.75 microns. Alternatively, or at least in addition to, at least about 75%, or at least about 85%, or at least about 90% of the droplet size of the discontinuous phase oil in the oil-in-water emulsion have a size of about 15 microns to about 35 microns, about 5 microns to about 20 microns, about 10 microns to about 15 microns, or about 1 microns to about 10 microns. Any combination of the above percentages and droplet sizes may be used to define oil droplets in a composition of the present invention.

In one embodiment, the emulsion composition of the present invention suitably have at least one of the following characteristics: a D50 of mean droplet diameter of about 0.1 micron to about 35 microns when measured at 2-8 degrees C.; a D50 of mean droplet diameter of about 0.1 micron to about 35 microns when measured at 25 degrees C. and 60% RH, a D50 of mean droplet diameter of about 0.1 micron to about 35 microns when measured at 30 degrees C. at 6 months; or a D50 mean droplet diameter of about 0.1 micron to about 35 microns when measured at 30 degrees C. at 6 months.

In another embodiment, a topical pharmaceutical emulsion composition comprising the active ingredient 3,5-Dihydroxy-4-isopropyl-trans-stilbene or a pharmaceutically acceptable salt thereof, an oil phase, a water phase, a surfactant, and an antioxidant, and wherein the average droplet size of the oil phase is about 0.1 micron to about 35 microns and optionally the emulsion is homogeneous. In another embodiment, the active is solubilized in the oil phase.

In another embodiment, a topical pharmaceutical emulsion composition comprising the active ingredient 3,5-Dihydroxy-4-isopropyl-trans-stilbene or a pharmaceutically acceptable salt thereof, an oil phase, a water phase, a surfactant, and an antioxidant, and wherein the average droplet size of the oil phase is about 10 microns to about 35 microns; and wherein the emulsion is homogeneous, and the active is solubilized in the oil phase.

In another embodiment, a topical pharmaceutical emulsion composition comprising the active ingredient 3,5-Dihydroxy-4-isopropyl-trans-stilbene or a pharmaceutically acceptable salt thereof, an oil phase, a water phase, a surfactant, and an antioxidant, and wherein the average droplet size of the oil phase is about 0.1 micron to about 25 microns and optionally the emulsion is homogeneous. In another embodiment, the active is solubilized in the oil phase.

In another embodiment, a topical pharmaceutical emulsion composition comprising the active ingredient 3,5-Dihydroxy-4-isopropyl-trans-stilbene or a pharmaceutically acceptable salt thereof, an oil phase, a water phase, a surfactant, and an antioxidant, and wherein the average droplet size of the oil phase is about 1 micron to about 25 microns; and wherein the emulsion is homogeneous, and the active is solubilized in the oil phase.

In another embodiment, a topical pharmaceutical emulsion composition comprising the active ingredient 3,5-Dihydroxy-4-isopropyl-trans-stilbene or a pharmaceutically acceptable salt thereof, an oil phase, a water phase, a surfactant, and an antioxidant, and wherein the average droplet size of the oil phase is about 0.1 micron to about 5 microns and optionally the emulsion is homogeneous. In another embodiment, the active is solubilized in the oil phase.

In another embodiment, a topical pharmaceutical emulsion composition comprising the active ingredient 3,5-Dihydroxy-4-isopropyl-trans-stilbene or a pharmaceutically acceptable salt thereof, an oil phase, a water phase, a surfactant, and an antioxidant, and wherein the average droplet size of the oil phase is about 0.1 micron to about 5 microns to; and wherein the emulsion is homogeneous, and the active is solubilized in the oil phase.

In another embodiment, a topical pharmaceutical emulsion composition comprising the active ingredient 3,5-Dihydroxy-4-isopropyl-trans-stilbene or a pharmaceutically acceptable salt thereof, an oil phase, a water phase, a surfactant, and an antioxidant, and wherein the average droplet size of the oil phase is about 0.1 micron to about 1 micron and optionally the emulsion is homogeneous. In another embodiment, the active is solubilized in the oil phase.

In another embodiment, a topical pharmaceutical emulsion composition comprising the active ingredient 3,5-Dihydroxy-4-isopropyl-trans-stilbene or a pharmaceutically acceptable salt thereof, an oil phase, a water phase, a surfactant, and an antioxidant, and wherein the average droplet size of the oil phase is about 0.1 micron to about 1 micron; and the emulsion is homogeneous, and the active is solubilized in the oil phase.

The terms “emulsion” and “oil-in-water emulsion” as used herein, and unless otherwise stated or understood from the context used, refers to a colloidal dispersion system in which liquid oil is dispersed as droplets (the discrete phase, also referred to as “the discontinuous non-aqueous phase” or “the discontinuous phase”) in a continuous aqueous medium (the continuous phase, also referred to as “the continuous aqueous phase” or “the continuous phase”). In some embodiments, at least 50% of the active ingredient (w/w) is dissolved and remains in the emulsion. In some embodiments, at least 75% of the active ingredient (w/w) is dissolved and remains in the emulsion. In certain embodiments, as is further described herein, greater than 85% of the active ingredient is present in the discontinuous phase. Emulsions described herein are multiphase systems and are not a single phase system. If a gelling agent is added the emulsion created is often referred to as an emugel. The emulsions described herein are not foams and do not include foaming agents. Further, the emulsions described herein are prepared using additional energy, for example, through homogenization and are not self-assembling.

The singular forms “a,” “an,” and “the” include plural referents unless the context clearly dictates otherwise. Thus, for example, reference to an “excipient” includes a single excipient as well as two or more of the same or different excipients, and the like.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. Furthermore, the phrases “less than about” a value or “greater than about” a value should be understood in view of the definition of the term “about” provided herein.

The terms “administer,” “administering” and “administration” as used herein refer to either directly administering a compound (also referred to as an agent of interest) or pharmaceutically acceptable salt of the compound (agent of interest) or a composition to a subject.

The transitional term “comprising,” which is synonymous with “including,” “containing,” or “characterized by,” is inclusive or open-ended and does not exclude additional, un-recited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed subject matter. In some embodiments or claims where the term comprising is used as the transition phrase, such embodiments can also be envisioned with replacement of the term “comprising” with the terms “consisting of” or “consisting essentially of.”

The phrase “pharmaceutically acceptable” is employed herein to refer to those agents of interest/compounds, salts, compositions, dosage forms, etc., which are within the scope of sound medical judgment suitable for use in contact with the tissues of human beings and/or other mammals without excessive toxicity, irritation, allergic response, or other problem or complication, commensurate with a reasonable benefit/risk ratio. In some aspects, pharmaceutically acceptable means approved by a regulatory agency of the federal or a state government, or listed in the U.S. Pharmacopeia or other generally recognized pharmacopeia for use in mammals (e.g., animals), and more particularly, in humans.

The terms “patient” and “subject” are interchangeable and may be taken to mean any living organism which may be treated with compounds or compositions provided for herein. As such, the terms “patient” and “subject” may comprise, but is not limited to, any non-human mammal, primate or human. In some embodiments, the patient or subject is an adult, child, or infant. In some embodiments, the patient or subject is a human.

The term “composition” as used herein refers to a combination or a mixture of two or more different ingredients, components, or substances; e.g., a combination of antioxidants.

The term “treating” as used herein refers to methods of treating a skin disorder, and generally includes the administration of a compound or composition which reduces the frequency of, or delays the onset of, symptoms of a medical condition. This can include reversing, reducing, or arresting the symptoms, clinical signs, and underlying pathology of a condition in a manner to improve or stabilize a subject's condition.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

### 3,5-Dihydroxy-4-isopropyl-trans-stilbene

In an embodiment, the 3,5-Dihydroxy-4-isopropyl-trans-stilbene or a pharmaceutically acceptable salt thereof is present in the emulsion composition in an amount from about 0.01% to about 5% by weight, such as from about 0.05% to about 2% by weight, about 0.1% to about 1% by weight, or about 0.1% to about 5% by weight, based on the total weight of the composition. In another embodiment, the 3,5-Dihydroxy-4-isopropyl-trans-stilbene or a pharmaceutically acceptable salt thereof is present in an amount from about 0.1% to about 1.0% by weight, based on the total weight of the composition. In one embodiment, the 3,5-Dihydroxy-4-isopropyl-trans-stilbene or a pharmaceutically acceptable salt thereof is present in an amount of about 0.25% to about 0.50% by weight. In one embodiment, the 3,5-Dihydroxy-4-isopropyl-trans-stilbene or a pharmaceutically acceptable salt thereof is present in an amount of about 0.1% to about 5% by weight. In an embodiment, the 3,5-Dihydroxy-4-isopropyl-trans-stilbene or a pharmaceutically acceptable salt thereof is present in an amount of about 0.25%, 0.30%, 0.40%, 0.50%, 0.75%, 1% or 2% by weight, based on the total weight of the composition.

In certain embodiments, the topical pharmaceutical oil-in-water emulsion composition comprises: about 0.1% to about 5% by weight of 3,5-dihydroxy-4-isopropyl-trans-stilbene or a pharmaceutically acceptable salt thereof, mineral oil in an amount of about 5% to about 15% by weight, based on the total weight of the composition, a co-solvent in an amount of about 1% to about 30% by weight, based on the total weight of the composition, a water phase in an amount of 25% to about 85% by weight, based on the total weight of the composition, a surfactant in an amount of about 0.1% to about 20% by weight, based on the total weight of the composition, comprising a mixture of polysorbate 80, nonionic emulsifying wax (Polawax NF), Steareth 2, and Steareth 20, and an antioxidant.

In certain embodiments, the topical pharmaceutical oil-in-water emulsion composition comprises: about 0.1% to about 5% by weight of 3,5-dihydroxy-4-isopropyl-trans-stilbene or a pharmaceutically acceptable salt thereof, an oil phase in an amount of about 5% to about 45% by weight, based on the total weight of the composition, a water phase in an amount of about 25% to about 85% by weight, based on the total weight of the composition, a surfactant in an amount of about 0.1% to about 20% by weight, based on the total weight of the composition, an antioxidant, and a co-solvent in an amount of greater than 30% by weight, based on the total weight of the composition.

In certain embodiments, the topical pharmaceutical oil-in-water emulsion composition comprises: about 0.1% to about 5% by weight of 3,5-dihydroxy-4-isopropyl-trans-stilbene or a pharmaceutically acceptable salt thereof, an oil phase in an amount of about 5% to about 35% by weight, based on the total weight of the composition and is selected from the group consisting of cocoyl caprylocaprate, isostearyl isostearate, octyldodecanol, jajoba oil, sesame oil, walnut oil, isopropyl palmitate, isopropyl myristate, safflower oil, mineral oil, olive oil, almond oil, and combinations thereof, a water phase in an amount of about 25% to about 85% by weight, based on the total weight of the composition, a surfactant in an amount of about 0.1% to about 20% by weight, based on the total weight of the composition, and an antioxidant.

In certain embodiments, the topical pharmaceutical oil-in-water emulsion composition comprises: about 0.1% to about 5% by weight of 3,5-dihydroxy-4-isopropyl-trans-stilbene or a pharmaceutically acceptable salt thereof, an oil phase in an amount of about 5% to about 45% by weight, based on the total weight of the composition, a water phase in an amount of about 25% to about 85% by weight, based on the total weight of the composition, a surfactant in an amount of about 1% to about 20% by weight, based on the total weight of the composition and is selected from the group consisting of oleoyl polyoxyl-6 glycerides, PEG32 Stearate, PEG 75, PEG 20 cetostearyl ether, ceteareth-12, ceteareth-15, ethylene glycol palmitostearate, cetostearyl alcohol, glycerol monostearate, stearyl alcohol, cetyl alcohol, steareth-2, steareth-20, steareth-21, and combinations thereof, and an antioxidant.

### Oil Phase

The present topical pharmaceutical emulsion compositions comprise an oil phase. Suitably, the oil phase comprises one or more oils and/or fats.

Exemplary oils and fats include fatty acids, esters, esters of glycerin, fatty alcohols, waxes, sterols, unsaponifiables, siloxanes, silanes, lanolin, hydrocarbons, essential oils, vegetable oils, mineral oils, animal oils, edible oils, and mixtures thereof.

In an embodiment, the oil and/or fat is selected from the group consisting of an ester, an ester of glycerin, and mixtures thereof. In another embodiment, the oil and/or fat is at least an ester of glycerin.

In an embodiment, the oil phase comprises a fatty acid. Exemplary fatty acids include, but are not limited to, isostearic acid, oleic acid, stearic acid, linoleic acid, linolenic acid, myristic acid, palmitic acid, ricinoleic acid, arachidic acid, and mixtures thereof.

In an embodiment, the oil phase comprises an ester. Exemplary esters include, but are not limited to, coco-caprylate/caprate, diethyl sebacate, diisopropyl adipate, diisopropyl dilinoleate, ethyl oleate, ethylhexyl hydroxystearate, glycol distearate, glycol stearate, hydroxyoctacosanyl hydroxystearate, isopropyl isostearate (Crodamol IPIS), isopropyl myristate, isopropyl palmitate, isopropyl stearate, methyl glucose sesquistearate, methyl laurate, methyl salicylate, methyl stearate, myristyl lactate, octyl salicylate, oleyl oleate, PPG-20 methyl glucose ether distearate, propylene glycol diacetate, propylene glycol dicaprylate, propylene glycol monolaurate, propylene glycol monopalmitostearate, propylene glycol ricinoleate, triacetin, sucrose distearate, cocoyl caprylocaprate (Kollicream 3C), isostearyl isostearate (Crodamol ISIS), octyldodecanol (Kollicream OD), and mixtures thereof. In one embodiment, the ester is diethyl sebacate or diisopropyl adipate.

In an embodiment, the oil phase comprises an ester of glycerin. Exemplary esters of glycerin include, but are not limited to, caprylic/capric glycerides, caprylic/capric triglyceride, caprylic/capric/succinic triglyceride, capryl glucoside, cetearyl glucoside, cocoglycerides, decyl glucoside, lauryl glucoside, glyceryl citrate, glyceryl isostearate, glyceryl laurate, glyceryl monostearate, glyceryl oleate, glyceryl palmitate, glyceryl ricinoleate, glyceryl stearate, mono and diglyceride, PEG-12 glyceryl laurate, PEG-120 glyceryl stearate, polyglyceryl-3 oleate, polyoxyl glyceryl stearate, tallow glycerides, medium chain triglycerides (MCT), and mixtures thereof. In one embodiment, the oil phase of the emulsion comprises medium chain triglycerides. In one embodiment, the medium chain triglyceride carbon length is from C₆ to C₁₂. In another embodiment, the medium chain triglyceride carbon length is from C₆ to C₈.

In an embodiment, the oil phase comprises a fatty alcohol. Exemplary fatty alcohols include, but are not limited to, caprylic alcohol, decyl alcohol, lauryl alcohol, myristyl alcohol, behenyl alcohol, lanolin alcohol, arachidyl alcohol, oleyl alcohol (Kollicream OA), palm alcohol, isocetyl alcohol, cetyl alcohol, stearyl alcohol, and mixtures thereof. In one embodiment, the fatty alcohol is a mixture of cetyl alcohol and stearyl alcohol. Suitably, the ratio of cetyl alcohol to stearyl alcohol is about 2:1 to about 1:9.

In an embodiment, the oil phase comprises a wax. Exemplary waxes include, but are not limited to, beeswax, carnauba wax, dimethicone PEG-1 beeswax, dimethiconol beeswax, lanolin wax, microcrystalline wax, white wax, candelilla wax, paraffin wax, emulsifying wax, PEG-8 beeswax, yellow wax, cetyl esters wax, shellac wax, synthetic beeswax, and mixtures thereof.

In an embodiment, the oil phase comprises a sterol. Exemplary sterols include, but are not limited to, Brassica campestris sterols, C₁₀-C₃₀ cholesterol/lanosterol esters, canola sterols, cholesterol, lanolin cholesterols, Glycine soja sterols, PEG-20 phytosterol, phytosterols, and mixtures thereof.

In an embodiment, the oil phase comprises a siloxane and/or silane. Exemplary siloxanes and silanes include, but are not limited to, dimethicone, cyclomethicone, simethicone, phenyl dimethicone, cyclopentasiloxane, cyclotetrasiloxane, dimethyl siloxane, dimethicone cross polymer, and mixtures thereof.

In an embodiment, the oil phase comprises a hydrocarbon. Exemplary hydrocarbons include, but are not limited to, dodecane, petrolatum, squalane, squalene, paraffin, and mixtures thereof.

In an embodiment, the oil phase comprises an essential oil. Exemplary essential oils include, but are not limited to, primrose oil, rose oil, eucalyptus oil, borage oil, bergamot of 1, chamomile oil, citronella oil, lavender oil, peppermint oil, pine oil, pine needle oil, spearmint oil, tea tree oil, wintergreen oil, jajoba oil, and mixtures thereof.

In an embodiment, the oil phase comprises a vegetable oil. Exemplary vegetable oils include, but are not limited to, almond oil, aniseed oil, canola oil, castor oil, coconut oil, corn oil, avocado oil, cottonseed oil, olive oil, palm kernel oil, peanut oil, sunflower oil, safflower oil, soybean oil, sesame oil, walnut oil, and mixtures thereof.

In an embodiment, the oil phase may comprise a mineral oil. Exemplary mineral oils include, but are not limited to, mineral oil and light mineral oil. If the oil phase comprises mineral oil, there is another oil phase component present in the formulation. In one embodiment, the second oil phase component will not be petrolatum. In one embodiment, the second oil phase component will not be a petrolatum derivative. In one embodiment, the emulsion composition comprises an oil phase that is substantially free from mineral oil. In another embodiment, the oil phase is substantially free from petrolatum. In another embodiment, the oil phase is substantially free from a petrolatum derivative. In another embodiment, the emulsion composition comprises an oil phase that is substantially free from mineral oil and petrolatum. In another embodiment, the emulsion composition comprises an oil phase that is substantially free from mineral oil, petrolatum and a petrolatum derivative.

In an embodiment, the oil phase comprises an edible oil. Exemplary edible oils include, but are not limited to, cinnamon oil, clove oil, lemon oil and peppermint oil, and mixtures thereof.

In certain embodiments, the oil phase comprises medium chain triglycerides (MCT), isostearyl isostearate (Crodamol ISIS), and isopropyl palmitate (IPP).

In certain embodiments, the oil phase comprises medium chain triglycerides (MCT), isopropyl myristate (IPM), and safflower oil.

In certain embodiments, the oil phase comprises isopropyl palmitate (IPP) and mineral oil.

In certain embodiments, the oil phase comprises medium chain triglycerides (MCT) and mineral oil.

In certain embodiments, the oil phase comprises jajoba oil and olive oil.

In certain embodiments, the oil phase comprises jajoba oil and almond oil.

In certain embodiments, the oil phase comprises diisopropyl adipate (DIPA) and mineral oil.

In one embodiment, the oil phase of the emulsion comprises an ester of glycerin which is a medium chain triglycerides (MCT). Suitably, the MCT is present in an amount from about 2% to about 30% by weight, about 5% to about 45% by weight, about 5% to about 35% by weight, based on the total weight of the composition. In certain embodiments, the MCT is present in an amount of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% by weight, based on the total weight of the composition. In another embodiment, the oil phase of the emulsion comprises MCT in an amount from about 5% to about 30% by weight, based on the total weight of the composition. In another embodiment, the oil phase of the emulsion comprises MCT in an amount from about 5% to about 25% by weight, based on the total weight of the composition. In another embodiment, the oil phase of the emulsion comprises MCT in an amount from about 5% to about 20% by weight, based on the total weight of the composition. In another embodiment, the oil phase of the emulsion comprises MCT in an amount from about 5% to about 15% by weight, based on the total weight of the composition. In another embodiment, the MCT is present in an amount of about 10% by weight, based on the total weight of the composition.

In one embodiment, the oil phase comprises an oil and/or fat in an amount from about 2% to about 30% by weight, about 5% to about 45% by weight, about 5% to about 35% by weight, based on the total weight of the composition. In another embodiment, the oil phase comprises an oil and/or fat in an amount from about 5% to about 30% by weight, based on the total weight of the composition. In another embodiment, the oil phase comprises an oil and/or fat in an amount from about 5% to about 25% by weight, based on the total weight of the composition. In another embodiment, the oil phase comprises an oil and/or fat in an amount from about 5% to about 20% by weight, based on the total weight of the composition. In another embodiment, the oil phase comprises an oil and/or fat in an amount from about 5% to about 15% by weight, based on the total weight of the composition. In another embodiment, the oil phase comprises an oil and/or fat in an amount of about 10% by weight, based on the total weight of the composition. In certain embodiments, the oil phase comprises oil and/or fat in an amount of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% by weight, based on the total weight of the composition.

### Water Phase

The present topical pharmaceutical emulsion compositions comprise an aqueous or water phase comprising water. In one embodiment, the aqueous phase consists of water alone. In another embodiment, the aqueous phase includes one or more water miscible co-solvents. Suitably, the water phase is present in the composition in an amount from about 25% to about 85% by weight, based on the total weight of the composition. In an embodiment, the water phase is present in the composition in an amount from about 30% to about 80% by weight, based on the total weight of the composition. In another embodiment, the water phase is present in an amount from about 55% to about 75% by weight, based on the total weight of the composition.

In yet another embodiment, the ‘water phase’ is composed not of water but of aprotic solvents e.g., polar, leading to anhydrous formulations.

In one embodiment, the present invention provides for a topical pharmaceutical O/W emulsion composition comprising an effective amount of 3,5-Dihydroxy-4-isopropyl-trans-stilbene or a pharmaceutically acceptable salt thereof, an oil phase, a water phase, a surfactant, and an antioxidant, wherein the 3,5-Dihydroxy-4-isopropyl-trans-stilbene or pharmaceutically acceptable salt thereof is solubilized in the oil phase of the emulsion composition, and composition does not contain ethanol. In one embodiment, the present invention provides for a topical pharmaceutical O/W emulsion composition comprising an effective amount of 3,5-Dihydroxy-4-isopropyl-trans-stilbene or a pharmaceutically acceptable salt thereof, an oil phase, a water phase, a surfactant, and an antioxidant, wherein the 3,5-Dihydroxy-4-isopropyl-trans-stilbene or pharmaceutically acceptable salt thereof is solubilized in the oil phase of the emulsion composition, and composition does not contain isopropyl alcohol.

In one embodiment, the present invention provides for a topical pharmaceutical O/W emulsion composition comprising an effective amount of 3,5-Dihydroxy-4-isopropyl-trans-stilbene or a pharmaceutically acceptable salt thereof, an oil phase, a water phase, a surfactant, and an antioxidant, wherein the 3,5-Dihydroxy isopropyl-trans-stilbene or pharmaceutically acceptable salt thereof is solubilized in the oil phase of the emulsion composition, and composition contains less than 15% w/w short chain, lower molecular weight alcohols, such as a C1-4 alcohol. A short chain alcohol includes, ethanol, methanol, 1-propanol, 1,2-propanediol, and n-butanol. his includes, one embodiment, the amount of alcohol present in the composition is less than 10% w/w. In another embodiment, the amount of alcohol present in the composition is less than 5% w/w. In another embodiment, the amount of alcohol present in the composition is less than 2% w/w. In another embodiment, the amount of alcohol present in the composition is less than 1% w/w. In another embodiment, the composition is free from alcohol. In another embodiment, the composition is formulated as a cream or lotion.

### Surfactant

The topical pharmaceutical emulsion compositions comprise a surfactant. In an embodiment, the surfactant is a mixture of two or more surfactants. As used herein, a surfactant is a compound that lowers the surface tension between two liquids or between a liquid and a solid. Surfactants may also act as detergents, wetting agents, emulsifiers, foaming agents, and dispersants. As further used herein, an emulsifier is equivalent to a surfactant.

Suitably, the surfactant is present in the composition in an amount from about 0.1% to about 20% by weight, such as from about 5% to about 15% by weight, about 1 to about 10%, or about 0.5% to about 5%, based on the total weight of the composition.

In certain embodiments, the surfactant is present in an amount of about 1%, about 1.1%, about 1.2%, about 1.3%, about 1.4%, about 1.5%, about 1.6%, about 1.7%, about 1.8%, about 1.9%, about 2%, about 2.7%, about 3%, about 4%, about 5%, about 6%, about 7%, about 7.2%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% by weight, based on the total weight of the composition.

A surfactant's hydrophilic/lipophilic balance (HLB) describes the surfactant's affinity toward water or oil. The HLB scale ranges from 1 (totally lipophilic) to 20 (totally hydrophilic), with 1.0 representing an equal balance of both characteristics. Lipophilic surfactants tend to form water-in-oil (w/o) emulsions, and hydrophilic surfactants tend to form oil-in-water (o/w) emulsions. The HLB of a blend of two surfactants equals the weight fraction of surfactant “A” times its HLB value plus the weight fraction of surfactant B times its HLB value (weighted average).

In one embodiment, the surfactant comprises one or more non-ionic surfactants. In another embodiment, the surfactant comprises two or more non-ionic surfactants and the weighted average of the HLB values of the two or more non-ionic surfactants is from about 10 to about 20. In yet another embodiment, the surfactant comprises two or more non-ionic surfactants and the weighted average of the HLB values of the two or more non-ionic surfactants is from about 1 to about 10.

Suitable non-ionic surfactants according to the invention include, but are not limited to, ethoxylated fatty alcohol ethers, PEG castor oils, PEG esters, propylene glycol esters, glyceryl esters and derivatives, polymeric ethers, sorbitan derivatives, fatty alcohols, emulsifying waxes, and mixtures thereof. In one embodiment, non-ionic surfactants include, ethoxylated fatty alcohol ethers, PEG esters, propylene glycol esters, glyceryl esters and derivatives, polymeric ethers, sorbitan derivatives, fatty alcohols, emulsifying waxes, and mixtures thereof. In another embodiment, non-ionic surfactants include, ethoxylated fatty alcohol ethers, propylene glycol esters, glyceryl esters and derivatives, polymeric ethers, sorbitan derivatives, fatty alcohols, emulsifying waxes, and mixtures thereof.

In an embodiment, the non-ionic surfactant is an ethoxylated fatty alcohol ether. Exemplary ethoxylated fatty alcohol ethers include, but are not limited to, steareth-2, steareth-10, steareth-20, steareth-21, steareth-40, steareth-100, beheneth-10, ceteareth-2, ceteareth-3, ceteareth-5, ceteareth-6, ceteareth-10, ceteareth-12, ceteareth-15, ceteareth-20, ceteareth-21, ceteareth-22, ceteareth-25, ceteareth-30, ceteareth-31, ceteareth-32, ceteareth-33, ceteth-2, ceteth-10, ceteth-20, ceteth-23, choleth-24, isoceteth-20, laureth-2, laureth-3, laureth-4, laureth-5, laureth-9, laureth-10, laureth-12, laureth-15, laureth-20, laureth-21, laureth-22, laureth-23, nonoxynol-9, nonoxynol-15, octoxynol-1, octoxynol-9, oleth-2, oleth-5, oleth-10, oleth-20, C20-40 pareth-24, trideceth-10, PEG 20 cetostearyl ether (Cetamacrogol 1000), and mixtures thereof.

In an embodiment, the non-ionic surfactant is a PEG castor oil. Exemplary PEG castor oils include, but are not limited to, PEG-7 hydrogenated castor oil, PEG-25 hydrogenated castor oil, PEG-30 castor oil, PEG-33 castor oil, PEG-35 castor oil, PEG-36 castor oil, PEG-40 castor oil, PEG-40 hydrogenated castor oil, PEG-50 castor oil, PEG-54 hydrogenated castor oil, PEG-60 castor oil, PEG-60 hydrogenated castor oil, and mixtures thereof.

In an embodiment, the non-ionic surfactant is a PEG ester. Exemplary PEG esters include, but are not limited to, PEG-4 dilaurate, PEG-150 distearate, PEG-12 glyceryl laurate, PEG-120 glyceryl stearate, PEG-6 isostearate, PEG-4 laurate, PEG-8 laurate, PEG-20 methyl glucose sesquistearate, PEG-5 oleate, PEG-6 oleate, PEG-10 oleate, PEG-25 propylene glycol stearate, PEG-2 stearate, PEG-6 stearate, PEG-6-32 stearate, PEG-8 stearate, PEG-9 stearate, PEG-20 stearate, PEG-40 stearate, PEG-45 stearate, PEG-50 stearate, PEG-100 stearate, oleoyl polyoxyl-6 glycerides (Labrafil M 1944), PEG32 Stearate (Tefose 63), PEG 75 (Gelot 64), and mixtures thereof.

In an embodiment, the non-ionic surfactant is a propylene glycol ester. Exemplary propylene glycol esters include, but are not limited to, propylene glycol laurate, propylene glycol palmitostearate, propylene glycol ricinoleate, propylene glycol stearate, and mixtures thereof.

In an embodiment, the non-ionic surfactant is a glyceryl ester or derivative. Exemplary glyceryl esters and derivatives include, but are not limited to, glyceryl behenate, glyceryl dibchenate, glyceryl diolcate, glyceryl distearate, glyceryl isostearate, glyceryl laurate, glyceryl linoleate, glyceryl monostearate (also known as glycerol monostearate, glyceryl stearate, Kolliwax GMS II), glyceryl oleate, glyceryl palmitate, glyceryl ricinoleate, glyceryl stearate, PEG-23 glyceryl cocoate, PEG-6 caprylic/capric glycerides, PEG-7 glyceryl cocoate, polyglyceryl-10 diisostearate, polyglyceryl-2 diisostearate, polyglyceryl-3 diisostearate, polyglyceryl-6 diisostearate, PEG-12 glyceryl laurate, PEG-120 glyceryl stearate, ethylene glycol palmitostearate, and mixtures thereof.

In an embodiment, the non-ionic surfactant is a polymeric ether. Exemplary polymeric ethers include, but are not limited to, poloxamer 124, poloxamer 181, poloxamer 182, poloxamer 184, poloxamer 188, poloxamer 237, poloxamer 331, poloxamer 338, poloxamer 407, and mixtures thereof.

In an embodiment, the non-ionic surfactant is a sorbitan derivative. Exemplary sorbitan derivatives include, but are not limited to, polysorbate 20, polysorbate 40, polysorbate 60, polysorbate 65, polysorbate 80, sorbitan isostearate, sorbitan monolaurate, sorbitan monooleate, sorbitan monopalmitate, sorbitan monostearate (Span 60), sorbitan sesquioleate, sorbitan trioleate, sorbitan tristearate, sorbitan stearate/sorbitan laureate (Arlacel LC), and mixtures thereof.

In an embodiment, the non-ionic surfactant is a fatty alcohol. Exemplary fatty alcohols include, but are not limited to, isostearyl alcohol, caprylyl alcohol, decyl alcohol, lauryl alcohol, myristyl alcohol, behenyl alcohol, lanolin alcohol, arachidyl alcohol, oleyl alcohol, palm alcohol, isocetyl alcohol, cetyl alcohol, stearyl alcohol, cetearyl alcohol, and mixtures thereof. In one embodiment, the fatty alcohol is a mixture of cetyl alcohol and stearyl alcohol, known as cetearyl alcohol (also known as cetostearyl alcohol, Crodacol).

In an embodiment, the non-ionic surfactant is an emulsifying wax, e.g., a non-ionic emulsifying wax also known as emulsifying wax NF, or emulsifying wax BP. In an embodiment, the emulsifying wax is a mixture of cetearyl alcohol and polysorbate 60. In another embodiment, the emulsifying wax is a proprietary blend known as POLAWAX NF (Croda Inc, Edison, N.J., USA).

In one embodiment, the surfactant comprises one or more ethoxylated fatty alcohol ethers. In another embodiment, the ethoxylated fatty alcohol ether is a mixture of steareth-2 and steareth-20.

In one embodiment, the surfactant comprises a mixture of an ethoxylated fatty alcohol ether and a sorbitan derivative. In another embodiment, the mixture of an ethoxylated fatty alcohol ether and a sorbitan derivative is a mixture of steareth-2, steareth-20 and polysorbate 80.

In one embodiment, when there are 2 surfactants present in the formulation, each surfactant is present in an amount from about 0.5% to about 5% by weight, based on the total weight of the composition. In another embodiment, when there are 3 surfactants present in the formulation, each surfactant is present in an amount from about 0.5% to about 5% by weight, based on the total weight of the composition. Similarly, if there are 4 or more surfactants present they are each present in an amount from about 0.5% to about 5% by weight, based on the total weight of the composition. In one embodiment, when there are 2 surfactants present in the formulation, each surfactant is present in an amount from about 0.5% to about 15% by weight, based on the total weight of the composition. In another embodiment, when there are 3 surfactants present in the formulation, each surfactant is present in an amount from about 0.5% to about 15% by weight, based on the total weight of the composition. Similarly, if there are 4 or more surfactants present they are each present in an amount from about 0.5% to about 15% by weight, based on the total weight of the composition.

In one embodiment, the surfactant comprises a mixture of an ethoxylated fatty alcohol ether and an emulsifying wax. In another embodiment, the surfactant comprises a mixture of an ethoxylated fatty alcohol ether, a sorbitan derivative and an emulsifying wax. Suitably, the mixture of an ethoxylated fatty alcohol ether and an emulsifying wax is a mixture of steareth-2, steareth-20, and POLAWAX NF. Suitably, the mixture of an ethoxylated fatty alcohol ether, a sorbitan derivative and an emulsifying wax is a mixture of steareth-2, steareth-20, polysorbate 80 and POLAWAX NF. In an alternative embodiment, the surfactant comprises a mixture of an ethoxylated fatty alcohol ether and a fatty alcohol. Suitably, the mixture of an ethoxylated fatty alcohol ether and a fatty alcohol is a mixture of steareth-2, steareth-20, and cetearyl alcohol.

In another embodiment, the surfactant comprises a mixture of an ethoxylated fatty alcohol ether, a sorbitan derivative and a fatty alcohol. Suitably, the mixture of an ethoxylated fatty alcohol ether, a sorbitan derivative and a fatty alcohol is a mixture of steareth-2, steareth-20, polysorbate 80 and cetearyl alcohol.

In certain embodiments, the surfactant comprises cetostearyl alcohol, polysorbate 80, steareth 2, and steareth 20.

In certain embodiments, the surfactant comprises cetyl alcohol, polysorbate 80, steareth 2, and steareth 20.

In certain embodiments, the surfactant comprises Polawax, polysorbate 60, steareth 2, and steareth 21.

In certain embodiments, the surfactant comprises Polawax, glycerol monostearate, and PEG 20 cetostearyl ether.

In certain embodiments, the surfactant comprises Polawax, Tefose 63, and Labrifil M 1944.

In certain embodiments, the surfactant is not a phospholipid.

### Co-Solvent

The topical pharmaceutical emulsion compositions may further comprise a co-solvent. The function of the co-solvent is to help solubilize the 3,5-Dihydroxy-4-isopropyl-trans-stilbene or a pharmaceutically acceptable salt thereof in the oil phase and/or the water phase of the emulsion composition, as desired. An oil miscible co-solvent may be used to help solubilize the active ingredient in oil phase, and a water miscible co-solvent may be used to help solubilize the active ingredient in the water phase. In one embodiment, the co-solvent is used to help solubilize in the oil phase of the composition. In another embodiment, the co-solvent is used to help solubilize the 3,5-Dihydroxy-4-isopropyl-trans-stilbene or a pharmaceutically acceptable salt thereof in the water phase of the composition.

In an embodiment, the co-solvent is a mixture of two or more co-solvents.

Exemplary co-solvents include, but are not limited to, alcohols such as ethanol, including absolute ethanol, isopropanol, t-butyl alcohol, amyl alcohol, benzyl alcohol, cyclohexanedimethanol, diacetone alcohol, hexyl alcohol, tetrahydrofurfuryl alcohol, diethylene glycol monoethyl ether (also referred to as DGME, ethoxy diglycol, and sold under the tradename Transcutol), and mixtures thereof. Exemplary co-solvents include, but are not limited to, carboxylic acids such as acetic acid, multi carboxylic acid, and mixtures thereof. Exemplary co-solvents include, but are not limited to, diols such as 1,2-hexanediol, butylene glycol, diethylene glycol, di propylene glycol, ethyl hexanediol, ethylene glycol, hexylene glycol, pentylene glycol, propylene glycol, propylene glycol monolaurate, tetraethylene glycol, triethylene glycol, tripropylene glycol, polyethylene glycol, polyethylene glycol 400, and mixtures thereof. Exemplary co-solvents include, but are not limited to, polyols such as butanetriol, glycerol, 1,2,6-hexanetriol, and mixtures thereof. Exemplary co-solvents include, but are not limited to, esters such as dimethyl isosorbide, butyl stearate, C12-15 alkyl benzoate, C12-15 alkyl lactate, caprylic/capric tri glyceride, cetearyl ethylhexanoate, cetearyl isononanoate, cetyl octanoate, cetyl palmitate, coco-caprylate/caprate, cocoglycerides, decyl oleate, dibutyl adipate, dicaprylyl carbonate, diethylhexyl adipate, diethylhexyl succinate, diisopropyl adipate, dioctyl malate, di-PPG-2 myreth-10 adipate, di-PPG-3 myristyl ether adipate, ethyl oleate, ethylhexyl cocoate, ethylhexyl hydroxystearate, ethylhexyl palmitate, ethylhexyl pelargonate, ethylhexyl stearate, hexyl laurate, hexyldecyl laurate, hexyldecyl stearate, isocetyl stearate, isocetyl stearoyl stearate, isodecyl oleate, isopropyl myristate, isopropyl palmitate, isostearyl neopentanoate, isotridecyl isononanoate, lauryl lactate, myristyl lactate, myristyl myristate, octyldodecyl stearoyl stearate, oleyl erucate, oleyl oleate, pentaerythrityl tetracaprylate/caprate, pentaerythrityl tetraisostearate, PPG-2 myristyl ether propionate, propylene glycol dicaprylate/dicaprate, propylene glycol isostearate, propyl heptyl caprylate, stearyl octanoate dimethyl isosorbide, propylene carbonate, macrogol-15-hydroxystearate (sold under the name “Solutol HS 15” by BASF), hydrogenated castor oil, PEG 40 (sold under the name “Cremophor RH40” by BASF), PPG-15 stearyl ether (sold under the name “Arlamol E” by Uniquema), oleyl macrogol 6 glycerides (sold under the name “Labrafil M1944CS” by the Gattefosse), octyldodecanol (sold under the name “Eutanol G”), N-methyl-2-pyrrolidone (sold under the name “Pharmasolve”), and mixtures thereof.

The one or more solvent or co-solvent agents are generally present in the compositions of the invention in an amount that is, firstly, sufficient to obtain the required solubility of the active ingredient to be formulated and, secondly, compatible with the need to preserve sustained chemical stability of this same active ingredient.

In one embodiment, the co-solvent is propylene glycol. In another embodiment, the cosolvent is a mixture of propylene glycol and di ethylene glycol monoethyl ether.

In another embodiment, the co-solvent is PEG-400. In another embodiment, the co-solvent is a mixture of one or more of propylene glycol, transcutol, PEG 400, and glycerine.

While not definitive, in general water soluble co-solvents include, but are not limited to, alcohols such as ethanol, isopropanol, t-butyl alcohol, amyl alcohol, benzyl alcohol, cyclohexanedimethanol, diacetone alcohol, hexyl alcohol, tetrahydrofurfuryl alcohol and diethylene glycol monoethyl ether; carboxylic acids such as acetic acid or multi carboxylic acids; dials such as 1,2-hexanediol, butylene glycol, diethylene glycol, di propylene glycol, ethyl hexanediol, ethylene glycol, hexylene glycol, pentylene glycol, propylene glycol, tetraethylene glycol, triethylene glycol, tripropylene glycol and polyethylene glycol; polyols such as butanetriol, glycerol, 1,2,6-hexanetriol, macrogol-15-hydroxystearate (sold under the name “Solutol HS15” by BASF), PEG 40 (sold under the name “Cremophor RH40” by BASF), oleyl macrogol 6 glycerides (sold under the name “Labrafil M1944CS” by the Gattefosse), N-methyl-2-pyrrolidone (sold under the name “Pharmasolve”), and combinations and mixtures thereof.

While not definitive, in general, oil soluble co-solvents include, but are not limited to propylene glycol monolaurate, esters such as butyl stearate, C12-15 alkyl benzoate, C12-15 alkyl lactate, caprylic/capric triglyceride, cetearyl ethylhexanoate, cetearyl isononanoate, cetyl octanoate, cetyl palmitate, coco-caprylate/caprate, cocoglycerides, decyl oleate, dibutyl adipate, dicaprylyl carbonate, diethylhexyl adipate, di-ethylhexyl succinate, diisopropyl adipate, dioctyl malate, di-PPG-2 myreth-10 adipate, di-PPG-3 myristyl ether adipate, ethyl olcate, ethylhexyl cocoate, ethylhexyl hydroxystearate, ethylhexyl palmitate, ethylhexyl pelargonate, ethylhexyl stearate, hexyl laurate, hexyldecyl laurate, hexyldecyl stearate, isocetyl stearate, isocetyl stearoyl stearate, isodecyl oleate, isopropyl myristate, isopropyl palmitate, isostearyl neopentanoate, isotridecyl isononanoate, lauryl lactate, myristyl lactate, myristyl myristate, octyldodecyl stearoyl stearate, oleyl erucate, oleyl oleate, pentaerythrityl tetracaprylate/caprate, pentaerythrityl tetraisostearate, PPG-2 myristyl ether propionate, propylene glycol dicaprylate/dicaprate, propylene glycol isostearate, propylheptyl caprylate, and stearyl octanoatedimethyl isosorbide, propylene carbonate, hydrogenated castor oil, PPG-15 stearyl ether (sold under the name “Arlamol E” by Uniquema), octyldodecanol (sold under the name “Eutanol G”), and combinations and mixtures thereof.

Suitably, the total amount of one or more co-solvent is present in the composition in an amount greater than 30% by weight, such as from about 30% to about 50% by weight, about 30% to about 45%, or about 30% to about 40%, based on the total weight of the composition. In certain embodiments, the total amount of co-solvent is present in the composition in an amount of about 37%. In certain embodiments, the total amount of co-solvent is present in the composition in an amount of about 45%.

Suitably, the one or more co-solvent is present in the composition in an amount from about 1% to about 30% by weight, such as from about 5% to about 20% by weight, about 2% to about 10%, or about 10% to about 20%, based on the total weight of the composition. In certain embodiments, the co-solvent is present in the composition in an amount of about 2%. In certain embodiments, the co-solvent is present in the composition in an amount of about 10%.

### Antioxidant

The present topical pharmaceutical emulsion compositions comprise an antioxidant. In an embodiment, the antioxidant is a mixture of two or more antioxidants.

Exemplary antioxidants include, but are not limited to, butylated hydroxytoluene (BHT), butylated hydroxyanisole (BHA), tocopherol, propyl gallate, vitamin E TPGS and tert-Butylhydroquinone (TBHQ), and mixtures thereof. In an embodiment, the antioxidant is selected from the group consisting of butylated hydroxytoluene, propyl gallate and tocopherol, and mixtures thereof.

In one embodiment, the antioxidant is butylated hydroxytoluene. In another embodiment, the antioxidant is propyl gallate. In yet another embodiment, the antioxidant is a mixture of butylated hydroxytoluene and propyl gallate.

In an embodiment, the antioxidant is used in conjunction with a chelating agent to prevent or minimize metal-catalyzed reactions, such as reactions catalyzed by iron, nickel, copper, magnesium, calcium, zinc or aluminum ions.

Suitably, the antioxidant is present in the composition in an amount from about 0.001% to about 5% by weight, based on the total weight of the composition. In an embodiment, the antioxidant is present in an amount from about 0.01% to 1% by weight, such as about 0.05% by weight or about 0.1% by weight, based on the total weight of the composition.

### Dermatologically Acceptable Excipients

The present topical pharmaceutical emulsion compositions may further comprise one or more additional dermatologically acceptable excipients. Exemplary additional dermatologically acceptable excipients include, but are not limited to, a pH adjusting agent, a chelating agent, a preservative, a co-solvent, a penetration enhancer, a humectant, a thickening or gelling or viscosity building agent, a fragrance, a colorant, and mixtures thereof.

In one embodiment, the additional dermatologically acceptable excipient is a preservative. In one embodiment, the additional dermatologically acceptable excipient is at least one co-solvent. In one embodiment, the additional dermatologically acceptable excipient is selected from the group consisting of a pH adjusting agent, a chelating agent, a preservative and a co-solvent, and mixtures thereof. In another embodiment, the additional dermatologically acceptable excipient comprises a mixture of a pH adjusting agent, a chelating agent, a preservative and a co-solvent.

In an embodiment, the emulsion is an oil-in-water emulsion. In another embodiment, the emulsion is a water-in-oil emulsion.

Suitably, the emulsion may be formulated as a cream. The cream may be an oil-in-water cream or a water-in-oil cream. In one particular embodiment, the cream is an oil-in-water cream.

In another embodiment, the emulsion may be formulated as a lotion. The lotion may be an oil-in-water lotion or a water-in-oil lotion.

### pH Adjusting Agent

The present topical pharmaceutical emulsion compositions may further comprise a pH adjusting agent.

In an embodiment, the pH adjusting agent is an acid, an acid salt, or a mixture thereof. Suitably, the acid is selected from the group consisting of lactic acid, acetic acid, maleic acid, succinic acid, citric acid, benzoic acid, boric acid, sorbic acid, tartaric acid, edetic acid, phosphoric acid, nitric acid, sulphuric acid and hydrochloric acid, and mixtures thereof.

In another embodiment, the pH adjusting agent is a buffer. Suitably, the buffer is selected from the group consisting of citrate/citric acid, acetate/acetic acid, phosphate/phosphoric acid, propionate/propionic acid, lactate/lactic acid, ammonium/ammonia and edetate/edetic acid. In one embodiment, the pH adjusting agent is a buffer which is citrate/citric acid.

Suitably, the pH adjusting agent is present in the composition in an amount from about 0.01% to about 10% by weight, based on the total weight of the composition. In an embodiment, the pH of the composition is adjusted with a pH adjusting agent to a pH of from about 4 to about 7, such as from about 4.5 to about 6.5.

### Chelating Agents

The present topical pharmaceutical emulsion compositions may further comprise a chelating agent. In an embodiment, the chelating agent is a mixture of two or more chelating agents. As described herein, the compositions of the invention may comprise a mixture of a chelating agent and an antioxidant, where both excipients act to prevent or minimize oxidative degradation reactions in the composition.

Exemplary chelating agents include, but are not limited to, citric acid, glucuronic acid, sodium hexametaphosphate, zinc hexametaphosphate, ethylene diamine tetraacetic acid (EDTA), phosphonates, salts thereof, and mixtures thereof. Ethylene diamine tetraacetic acid is also known as edetic acid.

In one embodiment, the chelating agent is EDTA or a salt thereof, such as potassium, sodium or calcium salts of EDT A. In an embodiment, the EDTA or a salt thereof is disodium EDTA. In another embodiment, the chelating agent is citric acid. In yet another embodiment, the compositions of the invention comprise a mixture of a chelating agent and an antioxidant which is a mixture of EDTA or a salt thereof and propyl gal late. In a further embodiment, the compositions of the invention comprise a mixture of a chelating agent and an antioxidant which is a mixture of EDT A or a salt thereof and BHT. In one embodiment, the compositions of the invention comprise a mixture of a chelating agent and an antioxidant which is a mixture of disodium EDTA and BHT.

In yet a further embodiment, the compositions comprise a mixture of a chelating agent and an antioxidant which is a mixture of citric acid and propyl gallate. In an embodiment, the compositions of the invention comprise a mixture of a chelating agent and an antioxidant which is a mixture of citric acid and BHT.

Suitably, the chelating agent is present in the composition in an amount from about 0.01% to about 1% by weight, based on the total weight of the composition. In one embodiment, the chelating agent is present in the composition in an amount of about 0.1% by weight, based on the total weight of the composition.

### Preservatives

The present topical pharmaceutical emulsion compositions may further comprise a preservative. In an embodiment, the preservative is a mixture of two or more preservatives.

Exemplary preservatives include, but are not limited to, benzyl alcohol, imidazolidinyl urea, diazolidinyl urea, dichlorobenzyl alcohol, chloroxylenol, methyl paraben, ethyl paraben, propyl paraben, butyl paraben, phenoxyethanol, sorbic acid, benzoic acid, salts thereof, and mixtures thereof.

In an embodiment, the preservative is selected from the group consisting of benzyl alcohol, phenoxyethanol and benzoic acid, and mixtures thereof.

In one embodiment, the preservative is benzyl alcohol. In another embodiment, the preservative is phenoxyethanol. In yet another embodiment, the preservative is benzoic acid.

Suitably, the preservative is present in the composition in an amount from about 0.01% to about 2% by weight, based on the total weight of the composition. In one embodiment, the preservative is present in the composition in an amount of about 0.25% by weight, based on the total weight of the composition.

### Penetration Enhancer

The present topical pharmaceutical emulsion compositions may further comprise a penetration enhancer. In an embodiment, the penetration enhancer is a mixture of two or more penetration enhancers. The co-solvent or mixture of two or more co-solvents described herein may function as a penetration enhancer.

Exemplary penetration enhancers include, but are not limited to, fatty acids, fatty acid esters, fatty alcohols, pyrrolidones, sulfoxides, alcohols, diols and polyols, and mixtures thereof.

Exemplary fatty acids include, but are not limited to, oleic acid, capric acid, hexanoicacid, lauric acid, linoleic acid, linolenic acid, propionic acid and vaccenic acid, and mixtures thereof.

Exemplary fatty acid esters include, but are not limited to, glycerol monolaurate, glycerol monooleate, glycerol monolinoleate, isopropyl isostearate, isopropyl palmitate, isopropyl myristate, diethylsebacate, sorbitan monopalmitate, sorbitan oleate, sorbitan dilaurate, sorbitan trioleate, propylene glycol monolaurate and sucrose monolaurate, and mixtures thereof.

Exemplary fatty alcohols include, but are not limited to, cetyl alcohol, stearyl alcohol, decanol, tridecanol, lauryl alcohol, linolenyl alcohol and oleyl alcohol, and mixtures thereof.

Exemplary pyrrolidones include, but are not limited to, N-methyl pyrrolidone, 2-pyrrolidone and N-cyclohexyl-2-pyrrolidone, and mixtures thereof.

Exemplary sulfoxides include, but are not limited to, dimethyl sulfoxide and decylmethyl sulfoxide, and mixtures thereof.

Exemplary alcohols include, but are not limited to, lower (C1-C6) alcohols and diethylene glycol monoethyl ether, and mixtures thereof.

Exemplary diols include, but are not limited to, 1,2-hexanediol, butylene glycol, diethylene glycol, dipropylene glycol, ethyl hexanediol, ethylene glycol, hexylene glycol, pentylene glycol, propylene glycol, propylene glycol monolaurate, tetraethylene glycol, triethylene glycol, tripropylene glycol, polyethylene glycol and polypropylene glycol, and mixtures thereof.

Exemplary polyols include, but are not limited to, butanetriol, glycerol and 1,2,6-hexanetriol, and mixtures thereof.

Suitably, the penetration enhancer is present in the composition in an amount from about 0.5% to about 40% by weight, such as from about 1% to about 20% by weight or from about 5% to about 15% by weight, based on the total weight of the composition.

### Gelling Agent

The present topical pharmaceutical compositions may further comprise a gelling agent. In an embodiment, the gelling agent is a mixture of two or more gelling agents.

Exemplary gelling agents include, but are not limited to, agar, alginate, arabinoxylan, carrageenan, carboxymethylcellulose, hydroxyethylcellulose, hydroxypropylcellulose, hydroxypropyl methylcellulose, cellulose, curdlan, gelatin, gellan, β-glucan, tragacanth gum, guar gum, gum arabic, locust bean gum, pectin, starch, a carbomer, acrylate copolymers, silica, xanthan gum, salts thereof, or a combination or mixture thereof. In some embodiments, the gelling agent is the carbomer Carbopol 980 polymer. In some embodiments, the gelling agent is hydroxyethylcellulose (HEC).

Suitably, the gelling agent is present in the composition in an amount from about 0.1% to about 2% by weight, based on the total weight of the composition. In one embodiment, the gelling agent is present in the composition in an amount from about 0.2% to about 1% by weight, based on the total weight of the composition.

The 3,5-Dihydroxy-4-isopropyl-trans-stilbene or pharmaceutically acceptable salt thereof is applied to the patient for a dermatological disease or disorder in a dermatologically acceptable formulation. These formulations include any of the various known excipients which may be applied topically and which will permit even spreading of the active ingredient over the affected area, rapid drying, and/or increased penetration. Examples of suitable formulations will include solutions, milks, creams, ointments, gels, lotions, sprays, aerosols, foam, or suspensions.

### Cream

In one embodiment, the dermatologically acceptable formulation is a cream. In one embodiment, the cream is an oil-in-water cream. Suitably, the oil-in-water cream comprises an oil phase, a water phase, a surfactant and an antioxidant.

In one embodiment, the pH of the composition is adjusted to a pH of between about 2 to about 6, such as about 4 to about 6. In another embodiment, the pH of the composition is adjusted to a pH of between about 4.5 to about 5.5.

The composition may further comprise a co-solvent, a humectant, a chelating agent, a preservative, a fragrance, a colorant or a penetration enhancer, or a combination or mixture thereof.

### Viscosity

In certain embodiments, the viscosity of the topical pharmaceutical emulsion composition is from about 10,000 mPa·s to about 300,000 mPa·s, about 20,000 mPa·s to about 250,000 mPa·s, about 30,000 mPa·s to about 200,000 mPa·s, about 40,000 mPa·s to about 150,000 mPa·s, or about 50,000 mPa·s to about 100,000 mPa·s. In certain embodiments, the viscosity of the topical pharmaceutical emulsion composition is about 50,000 mPa·s.

The present invention also provides for a pharmaceutical product comprising a combination of therapeutic agents, for simultaneous, separate or sequential use in the treatment of conditions for which administration of 3,5-Dihydroxy-4-isopropyl-trans-stilbene or a pharmaceutically acceptable salt thereof is indicated.

In the context of this specification, the term “simultaneously” when referring to simultaneous administration of the relevant drugs means at exactly the same time, as would be the case, for example in embodiments where the drugs are combined in a single preparation. In other embodiments, “simultaneously” can mean one drug is administered a short duration after another, wherein “a short duration” means a duration which allows the drugs to have their intended synergistic effect.

In light of the foregoing, the present invention also relates to combination therapy, which may be a comprised of a simultaneous or co-administration, or serial administration of a combination of compounds or pharmaceutical compositions of the present invention with other active drug or therapeutic agents, and where such administration also is determined by one of ordinary skill in the art.

In such an aforementioned combination composition, the dosage form of the present invention, each of the active drug components are contained in effective dosage amounts.

In another aspect, the present invention relates to a combination therapy, where the second therapeutic agent may be administered before, concurrent with or after administration of the 3,5-Dihydroxy-4-isopropyl-trans-stilbene or a pharmaceutically acceptable salt thereof whether in the same formulation or in a separate formulation and whether or not the second therapeutic agent is administered by the same topical route, e.g., it may be given orally, intravenously intramuscularly, opthalmically, vaginally, rectally, etc.

In other words, the 3,5-Dihydroxy-4-isopropyl-trans-stilbene or a pharmaceutically acceptable salt thereof may be administered together, contemporaneously or sequentially in either order to the site of administration, or to a desired site of action. The order of administration is not deemed necessary, provided that if topically administered they are in contact at some point together at the site of administration or desired site of action. If both are present in the same vehicle they provide ease of administration to the patient, and perhaps increased compliance, but it is not required for the invention herein.

In another embodiment, the topical pharmaceutical compositions have greater than 90% of the original concentration of 3,5-Dihydroxy-4-isopropyl-trans-stilbene or a pharmaceutically acceptable salt thereof remaining after storage of the composition for 3 months at 40° C.

In one embodiment, the present invention provides a topical pharmaceutical emulsion composition comprising 3,5-Dihydroxy-4-isopropyl-trans-stilbene or a pharmaceutically acceptable salt thereof, an oil phase, a water phase, a surfactant, and an antioxidant, and wherein the emulsion is homogeneous. In another embodiment, the 3,5-Dihydroxy-4-isopropyl-trans-stilbene or pharmaceutically acceptable salt thereof is solubilized in the oil phase of the emulsion composition. In another embodiment, the composition is an oil-in-water cream.

In one embodiment, the present invention provides a topical pharmaceutical emulsion composition comprising 3,5-Dihydroxy-4-isopropyl-trans-stilbene or a pharmaceutically acceptable salt thereof, an oil phase, a water phase, a surfactant, an antioxidant and a preservative, and wherein the emulsion composition is homogeneous. In another embodiment, the 3,5-Dihydroxy-4-isopropyl-trans-stilbene or pharmaceutically acceptable salt thereof is solubilized in the oil phase of the emulsion composition. In another embodiment, the composition is an oil-in-water cream.

In one embodiment, the present invention provides a topical pharmaceutical emulsion composition comprising 3,5-Dihydroxy-4-isopropyl-trans-stilbene or a pharmaceutically acceptable salt thereof, an oil phase, a water phase, a surfactant, an antioxidant, a preservative and a co-solvent, and wherein the emulsion composition is homogeneous. In another embodiment, the 3,5-Dihydroxy-4-isopropyl-trans-stilbene or pharmaceutically acceptable salt thereof is solubilized in the oil phase of the emulsion composition. In another embodiment, the composition is an oil-in-water cream.

In one embodiment, the present invention provides a topical pharmaceutical emulsion composition comprising 3,5-Dihydroxy-4-isopropyl-trans-stilbene or a pharmaceutically acceptable salt thereof, an oil phase, a water phase, a surfactant, an antioxidant, a preservative, a co-solvent and a pH adjusting agent, and wherein the emulsion is homogeneous. In another embodiment, the 3,5-Dihydroxy-4-isopropyl-trans-stilbene or pharmaceutically acceptable salt thereof is solubilized in the oil phase of the emulsion composition. In another embodiment, the composition is an oil-in-water cream.

Suitably, the 3,5-Dihydroxy-4-isopropyl-trans-stilbene or pharmaceutically acceptable salt thereof is solubilized in the oil phase of the emulsion composition. Suitably, the composition is an oil-in-water cream.

In all of the compositions described herein, the amount of 3,5-Dihydroxy-4-isopropyl-trans-stilbene or a pharmaceutically acceptable salt thereof which may be present in a composition may range from about 0.25% to about 2% by weight of composition. In one embodiment, the amount may be 0.25%, 0.3%, 0.4%, 0.5%, 0.75%, 1.0%, 1.25%, 1.5%, 1.75% or 2.0% by weight, based on the total weight of the composition. In another embodiment, the amount is about 0.25% to about 0.50% by weight, based on the total weight of the composition. In another embodiment, the amount is about 0.25% by weight, based on the total weight of the composition. In another embodiment, the amount is about 0.50% by weight, based on the total weight of the composition. In another embodiment, the amount is about 0.75% by weight, based on the total weight of the composition. In another embodiment, the amount is about 1.0% by weight, based on the total weight of the composition.

While not wishing to be limited to this explanation it is believed that when the oil phase contains components that the active ingredient is not soluble in, such as mineral oil and/or petrolatum as exemplified herein by Formulations 1 and 12 the active ingredient is solubilized in the co-solvents, such as but not limited to propylene glycol and/or diethylene glycol monoethyl ether. Subsequently, when the aqueous phase is added to the oil phase, the active ingredient might be soluble in both the aqueous and/or the oil phase depending on where and how much of the co-solvent(s) partition into the system. Surprisingly, it was determined that formulations including greater than 30% of co-solvents allows for the active ingredient to be solubilized in either the oil phase or the water phase or both phases in the same formulation.

In certain embodiments, the topical emulsion composition comprises about 0.5% to about 1% 3,5-Dihydroxy-4-isopropyl-trans-stilbene; about 15.035% to about 45.735% of a water phase; a co-solvent containing about 7.765% to about 23.295% propylene glycol, about 7.765% to about 23.295% PEG 400, and about 7.765% to about 23.295% Transcutol P (diethylene glycol monoethyl ether); and optionally an oil phase containing about 5% to about 15% medium chain triglycerides; and optionally a surfactant containing about 3.6% to about 10.8% nonionic emulsifying wax (Polawax NF), about 0.75% to about 2.25% polysorbate 80, about 0.9% to about 2.7% Steareth 2 (Brij S2), and about 0.55% to about 1.65% Steareth 20 (Brij S20). In certain embodiments, the topical emulsion composition comprises the formulation as described as CR51ACT in Table 7.

In certain embodiments, the topical emulsion composition comprises about 0.5% to about 1% 3,5-Dihydroxy-4-isopropyl-trans-stilbene; about 18.915% to about 56.745% of a water phase; a co-solvent containing about 3.885% to about 11.655% propylene glycol, about 7.765% to about 23.295% PEG 400, and about 7.765% to about 23.295% Transcutol P (diethylene glycol monoethyl ether); and optionally an oil phase containing about 5% to about 15% medium chain triglycerides; and optionally a surfactant containing about 3.6% to about 10.8% nonionic emulsifying wax (Polawax NF), about 0.75% to about 2.25% polysorbate 80, about 0.9% to about 2.7% Steareth 2 (Brij S2), and about 0.55% to about 1.65% Steareth 20 (Brij S20). In certain embodiments, the topical emulsion composition comprises the formulation as described as CR47ACT in Table 7.

In certain embodiments, the topical emulsion composition comprises about 0.5% to about 1% 3,5-Dihydroxy-4-isopropyl-trans-stilbene; about 15.035% to about 45.735% of a water phase; a co-solvent containing about 7.765% to about 23.295% propylene glycol, about 7.765% to about 23.295% PEG 400, about 3.885% to about 11.655% Transcutol P (diethylene glycol monoethyl ether), and about 3.85% to about 11.655% dimethyl isorbide (DMI); and optionally an oil phase containing about 5% to about 15% medium chain triglycerides; and optionally a surfactant containing about 3.6% to about 10.8% nonionic emulsifying wax (Polawax NF), about 0.75% to about 2.25% polysorbate 80, about 0.9% to about 2.7% Steareth 2 (Brij S2), and about 0.55% to about 1.65% Steareth 20 (Brij S20). In certain embodiments, the topical emulsion composition comprises the formulation as described as CR51ACT in Table 7.

The embodiments described herein do not include the formulations of CR48, CR49, CR52, CR53, or CR54, which are set forth in Tables 7, 9A-F of U.S. Provisional Application No. 63/043,360. Such formulations were not homogeneous or stable.

In certain embodiments, the topical emulsion composition comprises about 0.5% to about 1% 3,5-Dihydroxy-4-isopropyl-trans-stilbene; about 15.035% to about 45.735% of a water phase; a co-solvent containing about 7.765% to about 23.295% propylene glycol, about 7.765% to about 23.295% PEG 400, about 3.885% to about 11.655% Transcutol P (diethylene glycol monoethyl ether), and about 3.885% to about 11.655% dimethyl isorbide (DMI); and optionally an oil phase containing about 1% to about 3% medium chain triglycerides and about 4% to about 12% mineral oil; and optionally a surfactant containing about 3.6% to about 10.8% nonionic emulsifying wax (Polawax NF), about 0.75% to about 2.25% polysorbate 80, about 1.05% to about 3.15% Steareth 2 (Brij S2), and about 0.4% to about 1.2% Steareth 20 (Brij S20). In certain embodiments, the topical emulsion composition comprises the formulation as described as CR45ACT in Table 9A.

In certain embodiments, the topical emulsion composition comprises about 0.5% to about 1% 3,5-Dihydroxy-4-isopropyl-trans-stilbene; about 15.035% to about 45.735% of a water phase; a co-solvent containing about 7.765% to about 23.295% propylene glycol, about 7.765% to about 23.295% PEG 400, about 3.885% to about 11.655% Transcutol P (diethylene glycol monoethyl ether), and about 3.885% to about 11.655% dimethyl isorbide (DMI); and optionally an oil phase containing about 1% to about 3% medium chain triglycerides and about 4% to about 12% mineral oil; and optionally a surfactant containing about 3.6% to about 10.8% nonionic emulsifying wax (Polawax NF), about 0.75% to about 2.25% polysorbate 60, about 1.05% to about 3.15% Steareth 2 (Brij S2), and about 0.4% to about 1.2% Steareth 21 (Brij S721). In certain embodiments, the topical emulsion composition comprises the formulation as described as CR55ACT in Table 9A.

In certain embodiments, the topical emulsion composition comprises about 0.5% to about 1% 3,5-Dihydroxy-4-isopropyl-trans-stilbene; about 15.035% to about 45.735% of a water phase; a co-solvent containing about 7.765% to about 23.295% propylene glycol, about 7.765% to about 23.295% PEG 400, and about 7.765% to about 23.295% Transcutol P (diethylene glycol monoethyl ether); and optionally an oil phase about 5% to about 15% mineral oil; and optionally a surfactant containing about 3.6% to about 10.8% nonionic emulsifying wax (Polawax NF), about 0.75% to about 2.25% polysorbate 60, about 1.1% to about 3.3% Steareth 2 (Brij S2), and about 0.35% to about 1.05% Steareth 21 (Brij S721). In certain embodiments, the topical emulsion composition comprises the formulation as described as CR58ACT in Table 9B.

In certain embodiments, the topical emulsion composition comprises about 0.5% to about 1% 3,5-Dihydroxy-4-isopropyl-trans-stilbene; about 15.035% to about 45.735% of a water phase; a co-solvent containing about 7.765% to about 23.295% propylene glycol, about 7.765% to about 23.295% PEG 400, about 3.885% to about 11.655% Transcutol P (diethylene glycol monoethyl ether), and about 3.885% to about 11.655% dimethyl isorbide (DMI); and optionally an oil phase containing about 5% to about 15% mineral oil; and optionally a surfactant containing about 3.6% to about 10.8% nonionic emulsifying wax (Polawax NF), about 0.75% to about 2.25% polysorbate 60, about 1.1% to about 3.3% Steareth 2 (Brij S2), and about 0.35% to about 1.05% Steareth 21 (Brij S721). In certain embodiments, the topical emulsion composition comprises the formulation as described as CR59ACT in Table 9B.

In certain embodiments, the topical emulsion composition comprises about 0.5% to about 1% 3,5-Dihydroxy-4-isopropyl-trans-stilbene; about 15.035% to about 45.735% of a water phase; a co-solvent containing about 7.765% to about 23.295% propylene glycol, about 7.765% to about 23.295% PEG 400, and about 37.765% to about 23.295% Transcutol P (diethylene glycol monoethyl ether); a surfactant containing about 3.3% to about 9.9% cetostearyl alcohol and 2.5% to about 7.5% Arlacel 165 (sorbitan stearate/sorbitan laurate); and optionally an oil phase containing about 5% to about 15% mineral oil. In certain embodiments, the topical emulsion composition comprises the formulation as described as CR61ACT in Table 9C.

In certain embodiments, the topical emulsion composition comprises about 0.5% to about 1% 3,5-Dihydroxy-4-isopropyl-trans-stilbene; an oil phase containing about 5% to about 15% mineral oil; a water phase; a surfactant containing about 3.5% to about 10.5% stearyl alcohol, and about 3% to about 9% Arlacel 165 (glyceryl Stearate, PEG-100 stearate); a co-solvent containing about 7.505% to about 22.515% propylene glycol, about 7.505% to about 22.515% PEG 400, about 3.755% to about 11.265% Transcutol P (diethylene glycol monoethyl ether), about 3.755% to about 11.265% dimethyl isorbide (DMI), and about 0.05% to about 0.15% Carbopol 980 polymer. In certain embodiments, the topical emulsion composition comprises the formulation as described as CR77ACT in Table 9E.

In certain embodiments, the topical emulsion composition comprises about 0.5% to about 1% 3,5-Dihydroxy-4-isopropyl-trans-stilbene; an oil phase containing about 5% to about 15% mineral oil; a water phase; a surfactant containing about 3.5% to about 10.5% stearyl alcohol, and about 3% to about 9% Arlacel 165 (glyceryl Stearate, PEG-100 stearate); a co-solvent containing about 7.425% to about 22.275% propylene glycol, about 7.425% to about 22.275% PEG 400, about 3.715% to about 11.145% Transcutol P (diethylene glycol monoethyl ether), and about 3.715% to about 11.145% dimethyl isorbide (DMI). In certain embodiments, the topical emulsion composition comprises the formulation as described as CR78ACT in Table 9E.

In certain embodiments, the topical emulsion composition comprises about 0.5% to about 1% 3,5-Dihydroxy-4-isopropyl-trans-stilbene; an oil phase containing about 5% to about 15% mineral oil; a water phase; a surfactant containing about 3.5% to about 10.5% stearyl alcohol, and about 3% to about 9% Arlacel 165 (glyceryl Stearate, PEG-100 stearate); a co-solvent containing about 7.425% to about 22.275% propylene glycol, about 7.425% to about 22.275% PEG 400, about 3.715% to about 11.145% Transcutol P (diethylene glycol monoethyl ether), about 3.715% to about 11.145% dimethyl isorbide (DMI), and about 0.1% to about 0.3% hydroxyethyl cellulose. In certain embodiments, the topical emulsion composition comprises the formulation as described as CR76ACT in Table 9E.

The embodiments described herein do not include the formulations of CR40, CR41, CR42, CR43, CR44, CR46, CR56, CR57, CR60, CR62, CR67, CR68, CR69, CR74, or CR75, which are set forth in Tables 7, 9A-F of U.S. Provisional Application No. 63/043,360. Such formulations were not homogeneous or stable.

In certain embodiments, the topical emulsion composition comprises about 0.5% to about 1% 3,5-Dihydroxy-4-isopropyl-trans-stilbene; an oil phase containing about 5% to about 15% mineral oil; a water phase; a surfactant containing about 0.75% to about 2.25% polysorbate 80, about 3.6% to about 10.8% nonionic emulsifying wax (Polawax NF), about 1.1% to about 3.3% Steareth 2, and about 0.35% to about 1.05% Steareth 20; and a co-solvent containing about 5% to about 15% propylene glycol, and about 1% to about 3% Transcutol P (diethylene glycol monoethyl ether). In certain embodiments, the topical emulsion composition comprises the formulation as described as CR84 in Table 11.

In certain embodiments, the topical emulsion composition comprises about 0.5% to about 1% 3,5-Dihydroxy-4-isopropyl-trans-stilbene; an oil phase containing about 5% to about 15% mineral oil; a water phase; and a surfactant containing about 0.75% to about 2.25% polysorbate 80, about 3.6% to about 10.8% nonionic emulsifying wax (Polawax NF), about 1.1% to about 3.3% Steareth 2, and about 0.35% to about 1.05% Steareth 20. In certain embodiments, the topical emulsion composition comprises the formulation as described as CR85 in Table 11.

In certain embodiments, the topical emulsion compositions comprise the formulations as presented in Tables 7, 9A-9E, and 11.

In certain embodiments, the topical emulsion composition comprises about 0.5% to about 1% 3,5-Dihydroxy-4-isopropyl-trans-stilbene; an oil phase containing about 3% to about 9% medium chain triglycerides, about 0.5% to about 1.5% isopropyl palmitate (IPP), and about 1.5% to about 4.5% isostearyl isostearate (Crodamol ISIS); a water phase; optionally a co-solvent containing about 5% to about 15% propylene glycol, and about 1% to about 3% Transcutol P (diethylene glycol monoethyl ether); and optionally a surfactant containing about 3.6% to about 10.8% nonionic emulsifying wax (Polawax NF), about 0.75% to about 2.25% polysorbate 80 (Tween 80), about 0.9% to about 2.7% Steareth 2 (Brij S2), and about 0.55% to about 1.65% Steareth 20 (Brij S20). In certain embodiments, the topical emulsion composition comprises the formulation as described as CR27ACT in Table 14A.

In certain embodiments, the topical emulsion composition comprises about 0.5% to about 1% 3,5-Dihydroxy-4-isopropyl-trans-stilbene; an oil phase containing about 3% to about 9% medium chain triglycerides, about 1% to about 3% isopropyl myristate (IPM), and about 1% to about 3% safflower oil; a water phase; optionally a co-solvent containing about 5% to about 15% propylene glycol, and about 1% to about 3% Transcutol P (diethylene glycol monoethyl ether); and optionally a surfactant containing about 3.6% to about 10.8% nonionic emulsifying wax (Polawax NF), about 0.75% to about 2.25% polysorbate 80 (Tween 80), about 1% to about 3% Steareth 2 (Brij S2), and about 0.55% to about 1.65% Steareth 20 (Brij S20). In certain embodiments, the topical emulsion composition comprises the formulation as described as CR29ACT in Table 14B.

In certain embodiments, the topical emulsion composition comprises about 0.5% to about 1% 3,5-Dihydroxy-4-isopropyl-trans-stilbene; an oil phase containing about 2.5% to about 7.5% isopropyl palmitate (IPP), and about 2.5% to about 7.5% mineral oil; a water phase; optionally a co-solvent containing about 5% to about 15% propylene glycol, and about 1% to about 3% Transcutol P (diethylene glycol monoethyl ether); and optionally a surfactant containing about 3.6% to about 10.8% nonionic emulsifying wax (Polawax NF), about 0.75% to about 2.25% polysorbate 80 (Tween 80), about 1.1% to about 3.3% Steareth 2 (Brij S2), and about 0.35% to about 1.05% Steareth 20 (Brij S20). In certain embodiments, the topical emulsion composition comprises the formulation as described as CR30ACT in Table 14B.

In certain embodiments, the topical emulsion composition comprises about 0.5% to about 1% 3,5-Dihydroxy-4-isopropyl-trans-stilbene; an oil phase containing about 4% to about 12% medium chain triglycerides, and about 1% to about 3% mineral oil; a water phase; optionally a co-solvent containing about 5% to about 15% propylene glycol, and about 1% to about 3% Transcutol P (diethylene glycol monoethyl ether); and optionally a surfactant containing about 3.6% to about 10.8% nonionic emulsifying wax (Polawax NF), about 0.75% to about 2.25% polysorbate 80 (Tween 80), about 0.925% to about 2.775% Steareth 2 (Brij S2), and about 0.525% to about 1.575% Steareth 20 (Brij S20). In certain embodiments, the topical emulsion composition comprises the formulation as described as CR33ACT in Table 14C.

In certain embodiments, the topical emulsion composition comprises about 0.5% to about 1% 3,5-Dihydroxy-4-isopropyl-trans-stilbene; an oil phase containing about 1% to about 3% medium chain triglycerides, and about 4% to about 12% mineral oil; a water phase; optionally a co-solvent containing about 5% to about 15% propylene glycol, and about 1% to about 3% Transcutol P (diethylene glycol monoethyl ether); and optionally a surfactant containing about 3.6% to about 10.8% nonionic emulsifying wax (Polawax NF), about 0.75% to about 2.25% polysorbate 80 (Tween 80), about 1.05% to about 3.15% Steareth 2 (Brij S2), and about 0.4% to about 1.2% Steareth 20 (Brij S20). In certain embodiments, the topical emulsion composition comprises the formulation as described as CR34ACT in Table 14D.

In certain embodiments, the topical emulsion composition comprises about 0.5% to about 1% 3,5-Dihydroxy-4-isopropyl-trans-stilbene; an oil phase containing about 2.5% to about 7.5% medium chain triglycerides, and about 2.5% to about 7.5% mineral oil; a water phase; optionally a co-solvent containing about 5% to about 15% propylene glycol, and about 1% to about 3% Transcutol P (diethylene glycol monoethyl ether); and optionally a surfactant containing about 3.6% to about 10.8% nonionic emulsifying wax (Polawax NF), about 0.75% to about 2.25% polysorbate 80 (Tween 80), about 1% to about 3% Steareth 2 (Brij S2), and about 0.45% to about 1.35% Steareth 20 (Brij S20). In certain embodiments, the topical emulsion composition comprises the formulation as described as CR35ACT in Table 14D.

In certain embodiments, the topical emulsion composition comprises about 0.5% to about 1% 3,5-Dihydroxy-4-isopropyl-trans-stilbene; an oil phase containing about 2.5% to about 7.5% jajoba oil, and about 2.5% to about 7.5% olive oil; a water phase; optionally a co-solvent containing about 5% to about 15% propylene glycol, and about 1% to about 3% Transcutol P (diethylene glycol monoethyl ether); and optionally a surfactant containing about 3.6% to about 10.8% nonionic emulsifying wax (Polawax NF), about 0.75% to about 2.25% polysorbate 80 (Tween 80), about 0.9% to about 2.7% Steareth 2 (Brij S2), and about 0.55% to about 1.65% Steareth 20 (Brij S20). In certain embodiments, the topical emulsion composition comprises the formulation as described as CR36ACT in Table 14D.

In certain embodiments, the topical emulsion composition comprises about 0.5% to about 1% 3,5-Dihydroxy-4-isopropyl-trans-stilbene; an oil phase containing about 5% to about 15% olive oil; a water phase; optionally a co-solvent containing about 5% to about 15% propylene glycol, and about 1% to about 3% Transcutol P (diethylene glycol monoethyl ether); and optionally a surfactant containing about 3.6% to about 10.8% nonionic emulsifying wax (Polawax NF), about 0.2% to about 0.6% polysorbate 80 (Tween 80), about 1.75% to about 5.25% Steareth 2 (Brij S2), and about 0.25% to about 0.75% Steareth 20 (Brij S20). In certain embodiments, the topical emulsion composition comprises the formulation as described as CR38ACT in Table 14D.

In certain embodiments, the topical emulsion composition comprises about 0.5% to about 1% 3,5-Dihydroxy-4-isopropyl-trans-stilbene; an oil phase containing about 2.5% to about 7.5% jajoba oil, and about 2.5% to about 7.5% olive oil; a water phase; optionally a co-solvent containing about 5% to about 15% propylene glycol, and about 1% to about 3% Transcutol P (diethylene glycol monoethyl ether); and optionally a surfactant containing about 3.6% to about 10.8% nonionic emulsifying wax (Polawax NF), about 1.85% to about 5.55% Steareth 2 (Brij S2), and about 0.35% to about 1.05% Steareth 20 (Brij S20). In certain embodiments, the topical emulsion composition comprises the formulation as described as CR28ACT in Table 14B.

In certain embodiments, the topical emulsion composition comprises about 0.5% to about 1% 3,5-Dihydroxy-4-isopropyl-trans-stilbene; an oil phase containing about 2.5% to about 7.5% jajoba oil, and about 2.5% to about 7.5% almond oil; a water phase; optionally a co-solvent containing about 5% to about 15% propylene glycol, and about 1% to about 3% Transcutol P (diethylene glycol monoethyl ether); and optionally a surfactant containing about 3.6% to about 10.8% nonionic emulsifying wax (Polawax NF), about 1.95% to about 5.85% Steareth 2 (Brij S2), and about 0.25% to about 0.75% Steareth 20 (Brij S20). In certain embodiments, the topical emulsion composition comprises the formulation as described as CR32ACT in Table 14C.

The embodiments described herein do not include the formulations of CR23, CR24, CR25, CR37, or CR 39, which are set forth in Tables 7A-D, 9A-C, and 11A-F of U.S. Provisional Application No. 63/043,340. Such formulations are not homogeneous or stable.

In certain embodiments, the topical emulsion composition comprises about 0.5% to about 1% 3,5-Dihydroxy-4-isopropyl-trans-stilbene; a surfactant containing about 3.6% to about 10.8% Cetostearyl alcohol (Crodacol™ CS50), about 0.75% to about 2.25% polysorbate 80, about 0.5% to about 1.5% Steareth 2 (Brij S2), and about 0.95% to about 2.85% Steareth 20 (Brij S20); a water phase; and optionally an oil phase containing about 5% to about 15% medium chain triglycerides; and optionally a co-solvent containing about 5% to about 15% propylene glycol, and about 1% to about 3% Transcutol P (diethylene glycol monoethyl ether). In certain embodiments, the topical emulsion composition comprises the formulation as described as CR13ACT in Table 16A.

In certain embodiments, the topical emulsion composition comprises about 0.5% to about 1% 3,5-Dihydroxy-4-isopropyl-trans-stilbene; a surfactant containing about 3.6% to about 10.8% cetyl alcohol, about 0.75% to about 2.25% polysorbate 80, about 0.5% to about 1.5% Steareth 2 (Brij S2), and about 0.95% to about 2.85% Steareth 20 (Brij S20); a water phase; and optionally an oil phase containing about 5% to about 15% medium chain triglycerides; and optionally a co-solvent containing about 5% to about 15% propylene glycol, and about 1% to about 3% Transcutol P (diethylene glycol monoethyl ether). In certain embodiments, the topical emulsion composition comprises the formulation as described as CR14ACT in Table 16A.

In certain embodiments, the topical emulsion composition comprises about 0.5% to about 1% 3,5-Dihydroxy-4-isopropyl-trans-stilbene; a surfactant containing about 3.6% to about 10.8% nonionic emulsifying wax (Polawax NF), about 0.75% to about 2.25% polysorbate 60, about 0.9% to about 2.8% Steareth 2 (Brij S2), and about 0.95% to about 2.85% Steareth 21 (Brij S721); a water phase; and optionally an oil phase containing about 5% to about 15% medium chain triglycerides; and optionally a co-solvent containing about 5% to about 15% propylene glycol, and about 1% to about 3% Transcutol P (diethylene glycol monoethyl ether). In certain embodiments, the topical emulsion composition comprises the formulation as described as CR17ACT in Table 16B.

In certain embodiments, the topical emulsion composition comprises about 0.5% to about 1% 3,5-Dihydroxy-4-isopropyl-trans-stilbene; a surfactant containing about 3.6% to about 10.8% nonionic emulsifying wax (Polawax NF), about 0.85% to about 2.55% glycerol monostearate (Cithrol™ GMS 40), and about 1.35% to about 4.05% PEG 20 cetostearyl ether (Cetomacrogol 1000); a water phase; and optionally an oil phase containing about 5% to about 15% medium chain triglycerides; and optionally a co-solvent containing about 5% to about 15% propylene glycol, and about 1% to about 3% Transcutol P (diethylene glycol monoethyl ether). In certain embodiments, the topical emulsion composition comprises the formulation as described as CR21ACT in Table 16C.

In certain embodiments, the topical emulsion composition comprises about 0.5% to about 1% 3,5-Dihydroxy-4-isopropyl-trans-stilbene; a surfactant containing about 3.6% to about 10.8% nonionic emulsifying wax (Polawax NF), about 4% to about 12% Tefose 63 (PEG 6 stearate, ethylene glycol palmitostearate, PEG32 stearate), and about 1.35% to about 4.05% Labrafil M 1944 (oleoyl polyoxyl-6 glyceride); a water phase; and optionally an oil phase containing about 5% to about 15% medium chain triglycerides; and optionally a co-solvent containing about 5% to about 15% propylene glycol, and about 1% to about 3% Transcutol P (diethylene glycol monoethyl ether). In certain embodiments, the topical emulsion composition comprises the formulation as described as CR65ACT in Table 16C.

In certain embodiments, the topical emulsion composition comprises about 0.5% to about 1% 3,5-Dihydroxy-4-isopropyl-trans-stilbene; a surfactant containing about 3.6% to about 10.8% nonionic emulsifying wax (Polawax NF), about 6% to about 18% Tefose 63 (PEG 6 stearate, ethylene glycol palmitostearate, PEG32 stearate), and about 3% to about 9% Labrafil M 1944 (oleoyl polyoxyl-6 glyceride); a water phase; and optionally an oil phase containing about 5% to about 15% medium chain triglycerides; and optionally a co-solvent containing about 5% to about 15% propylene glycol, and about 1% to about 3% Transcutol P (diethylene glycol monoethyl ether). In certain embodiments, the topical emulsion composition comprises the formulation as described as CR73ACT in Table 16C.

In certain embodiments, the topical emulsion composition comprises about 0.5% to about 1% 3,5-Dihydroxy-4-isopropyl-trans-stilbene; a surfactant containing about 3.6% to about 10.8% nonionic emulsifying wax (Polawax NF), about 4% to about 12% Tefose 63 (PEG 6 stearate, ethylene glycol palmitostearate, PEG32 stearate), and about 2% to about 6% Labrafil M 1944 (oleoyl polyoxyl-6 glyceride); a water phase; and optionally an oil phase containing about 5% to about 15% medium chain triglycerides; and optionally a co-solvent containing about 5% to about 15% propylene glycol, and about 1% to about 3% Transcutol P (diethylene glycol monoethyl ether). In certain embodiments, the topical emulsion composition comprises the formulation as described as CR72ACT in Table 16C.

The embodiments described herein do not include the formulations of CR15, CR16, CR18, CR19, CR20, CR22, CR63, or CR66, which are set forth in Tables 7A-D, 9A-C, and 11A-F of U.S. Provisional Application No. 63/043,340. Such formulations are not homogeneous or stable.

In certain embodiments, the topical emulsion composition comprises about 0.5% to about 1% 3,5-Dihydroxy-4-isopropyl-trans-stilbene; an oil phase containing about 1.5% to about 4.5% medium chain triglycerides, and about 5% to about 15% diisopropyl adipate (DIPA); a water phase; and a surfactant containing about 10% to about 30% Tefose 63 (PEG 6 stearate, ethylene glycol palmitostearate, PEG32 stearate), and about 1.5% to about 4.5% Labrafil M 1944 (oleoyl polyoxyl-6 glyceride). In certain embodiments, the topical emulsion composition comprises the formulation as described as CR71ACT in Table 18B.

In certain embodiments, the topical emulsion composition comprises about 0.5% to about 1% 3,5-Dihydroxy-4-isopropyl-trans-stilbene; an oil phase containing about 1.5% to about 4.5% mineral oil, and about 5% to about 15% diisopropyl adipate (DIPA); a water phase; a surfactant containing about 2.275% to about 6.825% stearyl alcohol, about 3.35% to about 10.05% Kolliwax GMSII (glycerol monostearate), about 4.375% to about 13.125% PEG 100 stearate, and about 0.1% to about 0.3% Carbopol 980 polymer. In certain embodiments, the topical emulsion composition comprises the formulation as described as CR79ACT in Table 18C.

In certain embodiments, the topical emulsion composition comprises about 0.5% to about 1% 3,5-Dihydroxy-4-isopropyl-trans-stilbene; an oil phase containing about 1.5% to about 4.5% mineral oil, and about 5% to about 15% diisopropyl adipate (DIPA); a water phase; a surfactant containing about 2.275% to about 6.825% stearyl alcohol, about 3.35% to about 10.05% Kolliwax GMSII (glycerol monostearate), and about 4.375% to about 13.125% PEG 100 stearate; and a co-solvent containing about 5% to about 15% propylene glycol. In certain embodiments, the topical emulsion composition comprises the formulation as described as CR80ACT in Table 18C.

In certain embodiments, the topical emulsion composition comprises about 0.5% to about 1% 3,5-Dihydroxy-4-isopropyl-trans-stilbene; an oil phase containing about 2.5% to about 7.5% mineral oil, and about 2.5% to about 7.5% medium chain triglycerides; a water phase; a surfactant containing about 4% to about 12% cetyl alcohol, and about 2% to about 6% Gelot 64 (glyceryl monostearate/PEG 75); and a co-solvent containing about 5% to about 15% propylene glycol, and about 1% to about 3% Transcutol P. In certain embodiments, the topical emulsion composition comprises the formulation as described as CR64ACT in Table 18A.

In certain embodiments, the topical emulsion composition comprises about 0.5% to about 1% 3,5-Dihydroxy-4-isopropyl-trans-stilbene; an oil phase containing about 1.5% to about 4.5% mineral oil, and about 5% to about 15% diisopropyl adipate (DIPA); a water phase; a surfactant containing about 0.14% to about 0.42% stearyl alcohol, about 0.665% to about 1.995% polysorbate 60, and about 3.945% to about 11.835% Span 60 (sorbitan monostearate); and a co-solvent containing about 5% to about 15% propylene glycol. In certain embodiments, the topical emulsion composition comprises the formulation as described as CR70ACT in Table 18B.

The embodiments described herein do not include the formulations of CR40, CR41, CR42, CR43, CR44, CR46, CR56, CR57, CR60, CR62, CR63, CR67, CR68, CR69, CR74, or CR75, which are set forth in Tables 7A-D, 9A-C, and 11A-F of U.S. Provisional Application No. 63/043,340. Such formulations are not homogeneous or stable.

Alternatively, when discussed in a biological function, the term applied dose may be used. As used herein, applied dose is defined as the amount of drug product applied per body surface area, denoted in mg/cm² units. The amount of active ingredient delivered to the skin layers (epidermis or dermis) may be denoted in nanograms (ng) or micrograms (μg) per skin section or per cm². Alternatively, the amount of active ingredient delivered to epidermis or dermis may be denoted as % of the applied dose. The amount of active ingredient delivered to the receiving fluid may be denoted as cumulative amount in ng or ng/cm².

In embodiments described herein, the topical emulsion composition has an in vitro skin permeation profile of about 10 ng/cm² to about 65 ng/cm². In embodiments described herein, the topical emulsion composition has an in vitro skin permeation profile of about 9.66 ng/cm² to about 63.55 ng/cm². In embodiments described herein, the topical emulsion composition has an in vitro skin permeation profile of about 33.5 ng/cm² to about 43.1 ng/cm². In embodiments described herein, the topical emulsion composition has an in vitro skin permeation profile of about 10 ng/cm² to about 35 ng/cm². In embodiments described herein, the topical emulsion composition has an in vitro skin permeation profile of about 20 ng/cm² to about 65 ng/cm². In embodiments described herein, the topical emulsion composition has an in vitro skin permeation profile of about 20 ng/cm² to about 60 ng/cm². In embodiments described herein, the topical emulsion composition has an in vitro skin permeation profile of about 20 ng/cm² to about 55 ng/cm². In embodiments described herein, the topical emulsion composition has an in vitro skin permeation profile of about 20 ng/cm² to about 50 ng/cm². In embodiments described herein, the topical emulsion composition has an in vitro skin permeation profile of about 20 ng/cm² to about 45 ng/cm². In embodiments described herein, the topical emulsion composition has an in vitro skin permeation profile of about 20 ng/cm² to about 40 ng/cm². In embodiments described herein, the topical emulsion composition has an in vitro skin permeation profile of about 20 ng/cm² to about 35 ng/cm². In embodiments described herein, the topical emulsion composition has an in vitro skin permeation profile of about 20 ng/cm² to about 30 ng/cm².

In embodiments described herein, the topical emulsion composition has an in vitro penetration into the epidermis profile of about 500 ng to about 5,200 ng. In embodiments described herein, the topical emulsion composition has an in vitro penetration into the epidermis profile of about 631 ng to about 6,756 ng. In embodiments described herein, the topical emulsion composition has an in vitro penetration into the epidermis profile of about 2,368 ng to about 4,632 ng. In embodiments described herein, the topical emulsion composition has an in vitro penetration into the epidermis profile of about 500 ng to about 5,000 ng. In embodiments described herein, the topical emulsion composition has an in vitro penetration into the epidermis profile of about 500 ng to about 4,500 ng. In embodiments described herein, the topical emulsion composition has an in vitro penetration into the epidermis profile of about 500 ng to about 4,000 ng. In embodiments described herein, the topical emulsion composition has an in vitro penetration into the epidermis profile of about 500 ng to about 3,500 ng. In embodiments described herein, the topical emulsion composition has an in vitro penetration into the epidermis profile of about 500 ng to about 3,000 ng. In embodiments described herein, the topical emulsion composition has an in vitro penetration into the epidermis profile of about 500 ng to about 2,500 ng. In embodiments described herein, the topical emulsion composition has an in vitro penetration into the epidermis profile of about 500 ng to about 2,000 ng. In embodiments described herein, the topical emulsion composition has an in vitro penetration into the epidermis profile of about 500 ng to about 1,500 ng. In embodiments described herein, the topical emulsion composition has an in vitro penetration into the epidermis profile of about 500 ng to about 1,000 ng.

In embodiments described herein, the topical emulsion composition has an in vitro penetration into the dermis profile of about 1,800 ng to about 12,000 ng. In embodiments described herein, the topical emulsion composition has an in vitro penetration into the dermis profile of about 1,646 ng to about 9,056 ng. In embodiments described herein, the topical emulsion composition has an in vitro penetration into the dermis profile of about 5,246 ng to about 12,042 ng. In embodiments described herein, the topical emulsion composition has an in vitro penetration into the dermis profile of about 2,000 ng to about 6,000 ng. In embodiments described herein, the topical emulsion composition has an in vitro penetration into the dermis profile of about 2,000 ng to about 4,000 ng. In embodiments described herein, the topical emulsion composition has an in vitro penetration into the dermis profile of about 5,000 ng to about 12,000 ng. In embodiments described herein, the topical emulsion composition has an in vitro penetration into the dermis profile of about 5,000 ng to about 10,000 ng. In embodiments described herein, the topical emulsion composition has an in vitro penetration into the dermis profile of about 5,000 ng to about 8,000 ng.

In an embodiment, the emulsion composition comprises 3,5-Dihydroxy-4-isopropyl-trans-stilbene or a pharmaceutically acceptable salt thereof in a composition that has a human skin penetration measured in vitro of at least 0.01-10% of the applied dose of the active ingredient into the epidermis over a period of about 1 to about 72 hours. In another embodiment, the time period is from about 2 to about 24 hours. In another embodiment, the time period is about 1 to about 15 hours. The % of applied dose of the active ingredient may be from 0.01-10%, 0.01-5%, 0.01-3%, 0.4-2.3% w/w.

In one embodiment, the emulsion composition comprises the 3,5-Dihydroxy-4-isopropyl-trans-stilbene or a pharmaceutically acceptable salt thereof in a composition that has human skin penetration measured in vitro of at least 0.01-10% of the applied dose of the active ingredient into the dermis over a period of about 1 to about 72 hours. In another embodiment, the time period is from about 2 to about 24 hours. In another embodiment, the time period is about 6 to about 15 hours. The % of applied dose of the active ingredient may be from 0.01-7.5%, 0.01-5%, 0.01-3%, 0.3-1.7%. In an alternative embodiment, the applied dose measured in an amount of 1-2 μg/cm², e.g., 0.5% w/v.

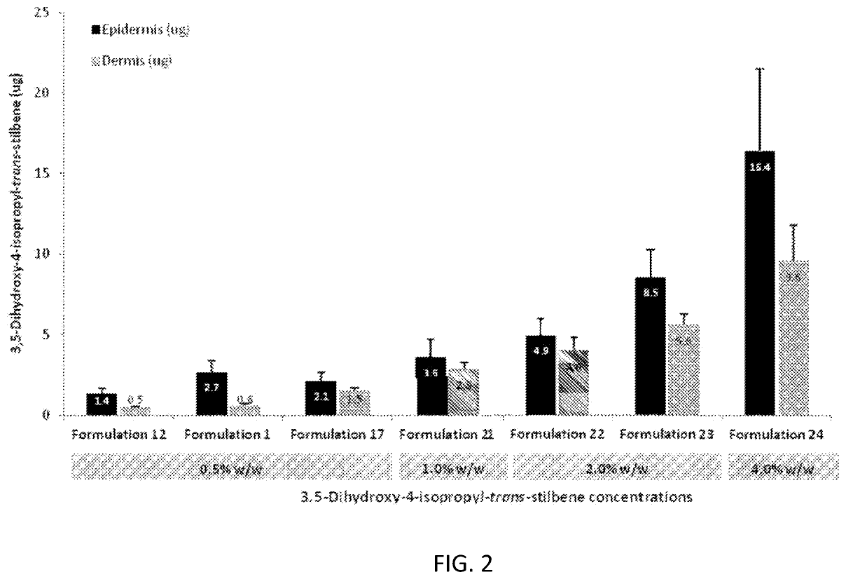

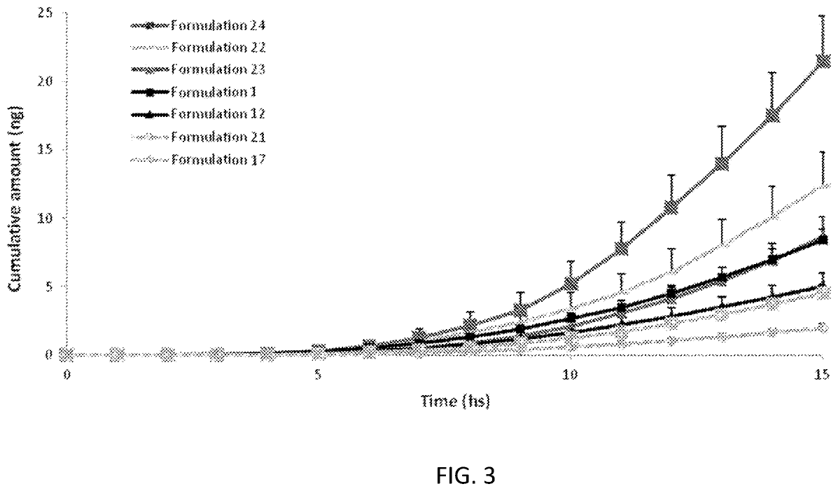

As illustrated in FIG. 2, Formulation 17 delivers a higher relative amount of the active ingredient into the dermis compared with Formulations 1 and 12. Conversely, Formulation 17 delivered comparatively less into the receiving fluid, as illustrated in FIG. 3. Formulations 1 and 12 were very comparable, with minor changes in buffer composition and preservative (Table 1). The in vitro human skin flux and dermal deposition results confirmed such similarities and enabled the extrapolation of further data comparison of any other formulation with Formulation 12 and, therefore with Formulation 1.

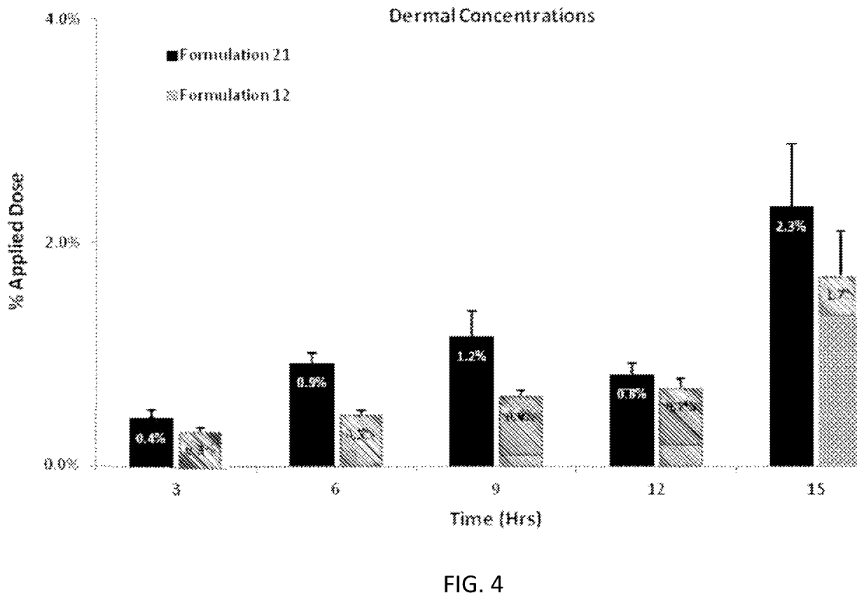

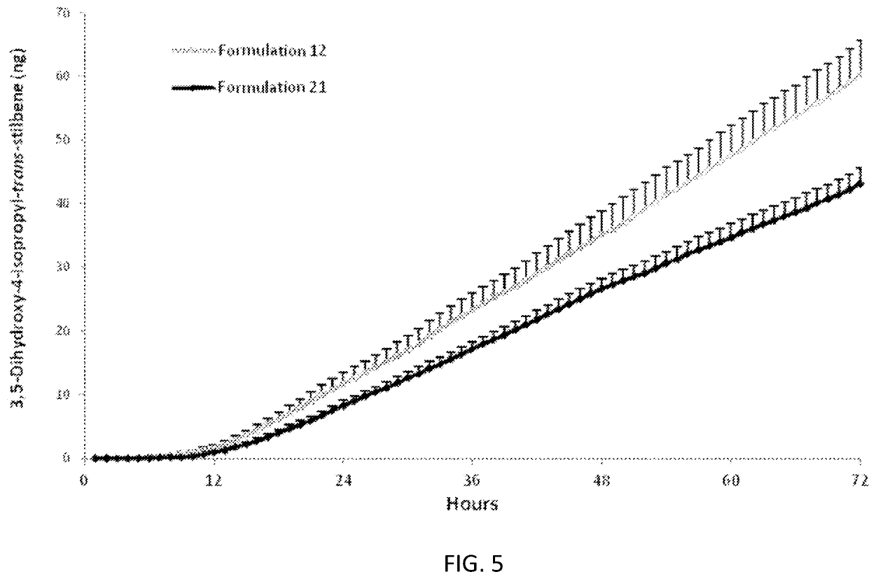

Although the two formulations shown in FIGS. 4 and 5 have differing amounts of active ingredient (Formulation 12 is 0.5% and Formulation 21 is 1%), the same conclusions can be reached when normalization is applied, e.g., Formulation 21 also shows a higher relative amount in the dermis as compared to the receiving fluid, which is measured as skin flux values and cumulative amounts, as normalized for applied dose. The cumulative amount represents how much active ingredient penetrates through the skin (500±100 μm), reaching the receiving fluid, over a specific period of time. The skin flux represents the slope of the cumulative amount curve, characterized as a linear phase and also referred as steady state (R²≥0.99), observed during the course of the experiment.

By converting the dermis amount from each time point (FIG. 4) from % of applied dose tong of the active ingredient and dividing this value by the respective cumulative amount in ng (FIG. 5) at 3, 6, 9, 12 and 15 hours, it was demonstrated that Formulation 21 promoted a targeted delivery to the dermis, resulting in less active ingredient penetrating the receiving fluid (unbound active ingredient penetrating deeper than 500±100 μm). The ratios dermis/cumulative amount in the receiving fluid (ng/ng) for Formulation 21 ranged from 9038.0 and 12937 at 3 hours and 6 hours, respectively, to 909.3 and 1044.0 at 12 hours and 15 hours, respectively. Formulation 12 showed a significantly different profile, with its ratios ranging from 7870.2 and 1428.7 at 3 hours and 6 hours, respectively, to 215.7 and 233.93 at 12 hours and 15 hours, respectively. Since Formulation 21 showed good dose proportionality at different active ingredient strengths ranging from 0.5% (Formulation 17) to 2.0% (Formulation 22) as observed in FIGS. 2 and 3, the ratio of dermis/cumulative amount explained above is valid to demonstrate the superiority of Formulation 21 over 12, despite their different strength (1.0% and 0.5%, respectively).

The lag phase for the active ingredient in the formulations ranged from 8 to 12 hours post-dosing—after this period of time the active ingredient reached its steady state and demonstrated a constant skin flux. The ratio dermis (ng)/skin flux (ng*cm²/hr) was used as an additional parameter to characterize the targeted delivery to the dermis observed for Formulation 21 in comparison to Formulation 12. From data shown on FIG. 5 the derived skin flux (slope with a R²≥0.99) for Formulations 21 and 12 are, respectively, 0.7251 ng*cm²/hr and 0.9935 ng*cm²/hr. Considering the different strengths of Formulations 21 and 12 (1.0% and 0.5%, respectively), the normalized skin flux values (skin flux divided by active ingredient strength) were 0.7251 ng*cm²/hr and 1.987 ng*cm²/hr, respectively. Using these normalized skin flux values and the dermis amounts converted tong (from FIG. 5) covering the steady state region (after 8 hours), the ratios dermis (ng)/normalized skin flux (ng*cm²/hr) were calculated. Formulation 21 demonstrated a selective delivery of the active ingredient to the dermis, with ratios of 1599.5, 1132.6 and 3200.7 at 9 hours, 12 hours and 15 hours, respectively. Formulation 12 showed a significantly different delivery profile, with ratios of 213.25, 108.57 and 117.73 at 9 hours, 12 hours and 15 hours, respectively.

One embodiment of the invention is a topical pharmaceutical emulsion composition comprising a therapeutically effective amount of 3,5-Dihydroxy-4-isopropyl-trans-stilbene or a pharmaceutically acceptable salt thereof, an oil phase, a water phase, a surfactant and an antioxidant, wherein the emulsion is homogeneous, and wherein the composition administered in an in vitro system results in a ratio of dermis amounts (ng) measured at steady state to normalized (by active strength) skin flux (ng*cm²/hr) from 1000 to 5000, using freshly excised abdominal human skin. In one embodiment, the 3,5-Dihydroxy-4-isopropyl-trans-stilbene or pharmaceutically acceptable salt thereof is solubilized in the oil phase of the emulsion composition.

In another embodiment of the invention there is a homogeneous topical pharmaceutical emulsion composition comprising an effective amount of 3,5-Dihydroxy-4-isopropyl-trans-stilbene or a pharmaceutically acceptable salt thereof, an oil phase, a water phase, a surfactant and an antioxidant, wherein the composition produces an area under the curve (AUC) AUC(0-tau) of less than 30 ng*h/mL, or less than 23.5 ng*h/mL or less than 16 ng*h/mL in a human upon administration to the skin in an amount not exceeding 35% Body Surface Area (BSA). In another embodiment, the amount is not exceeding 30% BSA.

In one embodiment, the AUC is at steady state. In another embodiment, the amount of body surface area (BSA) for which the drug is applied to is less than 50%, in another embodiment the amount is less than 35%, in another embodiment the amount is less than 30%. It is recognized that if the BSA is >10% than the AUC may be increased accordingly.

As used herein, the term “AUC(0-last)” means the area under the plasma concentration versus time curve, from time 0 to the last measurable concentration as calculated by the log-linear trapezoidal method.

As used herein, the term “AUC(0-12)” means the area under the plasma concentration versus time curve, from time 0 to the 12-hour time point, as calculated by the log-linear trapezoidal method.

As used herein, the term “AUC(0-tau)” means the area under the plasma concentration versus time curve from time 0 to end of the dosing interval, as calculated by the log-linear trapezoidal method.

In another embodiment of the invention there is a homogeneous topical pharmaceutical emulsion composition comprising an effective amount of 3,5-Dihydroxy-4-isopropyl-trans-stilbene or a pharmaceutically acceptable salt thereof, an oil phase, a water phase, a surfactant and an antioxidant, wherein the composition produces a Cₘₐₓ (maximum plasma level of drug) at steady state, and the amount of body surface area (BSA) for which the active ingredient (1% strength) is applied to is 15-35% and produces a Cₘₐₓ below 4 ng/ml and an AUC (0-8h) of no more than 16 ng·h/mL.

In patients with atopic dermatitis, when the active ingredient (0.5% strength) is applied once daily within the indicated BSA, 5-35% of patients produce plasma concentrations ranging from 40.8 ng/mL to 4170 ng/mL.

In patients with atopic dermatitis, when the active ingredient (0.5% strength) is applied twice daily within the indicated BSA, 5-35% of patients produce plasma concentrations ranging from 40.7 ng/mL to 10800 ng/mL.

In patients with atopic dermatitis, when the active ingredient (1% strength) is applied once daily within the indicated BSA, 5-35% of patients produce plasma concentrations ranging from 40.6 ng/mL to 1510 ng/mL.

In patients with atopic dermatitis, when the active ingredient (1% strength) is applied twice daily within the indicated BSA, 5-35% of patients produce plasma concentrations ranging from 41.4 ng/mL to 181000 ng/mL.

In one embodiment, there is a pharmaceutical composition for topical administration comprising the active ingredient 3,5-Dihydroxy-4-isopropyl-trans-stilbene present in an amount of about 1% w/w, or a pharmaceutically acceptable salt thereof, an oil phase, a water phase, a surfactant, and an antioxidant, and wherein the composition is homogeneous, and produces a plasma concentration ranging from about 40.6 ng/mL to about 1510 ng/mL in a human upon administration to the skin once daily.

In one embodiment, there is a pharmaceutical composition for topical administration comprising the active ingredient 3,5-Dihydroxy-4-isopropyl-trans-stilbene present in an amount of about 1% w/w, or a pharmaceutically acceptable salt thereof, an oil phase, a water phase, a surfactant, and an antioxidant, and wherein the composition is homogeneous, and produces a plasma concentration ranging from about 41.4 ng/mL to about 181000 ng/mL in a human upon administration to the skin twice daily.

In one embodiment, there is a pharmaceutical composition for topical administration comprising the active ingredient 3,5-Dihydroxy-4-isopropyl-trans-stilbene present in an amount of about 0.5% w/w, or a pharmaceutically acceptable salt thereof, an oil phase, a water phase, a surfactant, and an antioxidant, and wherein the composition is homogeneous, and produces a plasma concentration ranging from about 40.8 ng/mL to about 4170 ng/mL in a human upon administration to the skin once daily.

In one embodiment, there is a pharmaceutical composition for topical administration comprising the active ingredient 3,5-Dihydroxy-4-isopropyl-trans-stilbene present in an amount of about 0.5% w/w, or a pharmaceutically acceptable salt thereof, an oil phase, a water phase, a surfactant, and an antioxidant, and wherein the composition is homogeneous, and produces a plasma concentration ranging from about 40.6 ng/mL to about 1510 ng/mL in a human upon administration to the skin once daily.

| Formulation | Formulation 1a | Formulation 21 |
|---|---|---|
| Study | | |
| Age | 18-65 y | 18-65 y |
| Disease duration | ≥6 months diagnosis of AD | ≥6 months diagnosis of AD |
| Affected area | 1-10% BSA, excluding face, | 15-35% BSA, excluding scalp |
| | groin, scalp, genitals | |
| IGA (0-5) | 2-3 (mild-moderate) | ≥3 (moderate-severe) |
| Average % BSA treated | 2.7% (1-7%) | 19.8% (15-33%) |
| Study duration | 28 day | 21 day |
| Study treatment | Vehicle, 0.5%, 1% BID | 1%, 2% BID (no vehicle control) |
| Subject numbers | N = 12/10/12 (completed) | N = 62 (completed) |
| PK sampling scheme | Pre-dose, 1, 2, 4, 6, 8 h | Pre-dose, 1, 2, 4, 8, 10, 12, 24 h |
| | post-dose | post-dose |
| LLOQ | 0.1 ng/mL = 100 pg/mL | 40 pg/mL |
| 1% BID cohort data only | | |
| N | 12 | 6 |
| Day 1 Cmas (ng/mL) | 0.46 (0-3.32) | 1.23 (0.2-3.96) |
| Day 1 AUC(0-8) (ng · h/mL) | 1.22 (0-8.61) | 5.56 (1.05-15.14) |
| IGA score change from | 42% (0%-100%) | 55% (33%-75%) |
| baseline at Day 21 | | |
| BSA affected change from | 52% (−21%-100%) | 77% (56%-94%) |
| baseline at Day 21 | | |

Atopic Dermatitis is evaluated clinically and is based on historical features, morphology, distribution of skin lesions and associated clinical signs. Many formal sets of criteria have been developed to aid in classification. For measurement of disease severity, at least 28 different scales exist. Most commonly used include SCORAD index, the Eczema Area and Severity Index (EAST), the Investigators Global Assessment (IGA) and the Six area, Six sign Atopic Dermatitis (SASSAD) severity score. For purposes herein, the IGA scale or EASI scale will be referenced.

IGA is a static 5-point morphological assessment of overall disease severity, as determined by the physician, using the clinical characteristics of erythema, infiltration, papulation, oozing, and crusting as guidelines. The IGA is made without reference to the previous IGA scores.

IGA allows investigators to assess overall disease severity at one given time point, and it consists of a 5-point severity scale from clear to very severe disease (0=clear, 1=almost clear, 2=mild disease, 3=moderate disease, 4=severe disease). Some studies may use a 6-point scale of S=very severe disease). IGA uses clinical characteristics of erythema, infiltration, papulation, oozing and crusting as guidelines for the overall severity assessment. While it appears that IGA has not been validated as an outcome measure, IGA has been used to validate other outcome scales as one “gold standard.” While the combined use of IGA with another validated scale does not make IGA itself a stand-alone, validated instrument, IGA appears to correlate well with the EASI and is considered an instrument with reasonable face validity.

The EASI scoring system is a clinical tool for assessing the severity of AD that takes into account the overall severity of erythema, infiltration/papulation, excoriation, and lichenification, as well as the extent of BSA affected with AD. The 4 clinical signs are each graded on a 4-point scale (0 to 3) for each of the 4 specified body regions (head and neck, upper extremities, lower extremities, and trunk). EASI is also a static assessment made without reference to previous scores.

One embodiment of the invention is a method of treatment of atopic dermatitis (AD) in a patient in need thereof comprising administering to said patient a topical pharmaceutical emulsion composition comprising an effective amount of active 3,5-Dihydroxy-4-isopropyl-trans-stilbene or a pharmaceutically acceptable salt thereof, an oil phase, a water phase, a surfactant, and an antioxidant, wherein the emulsion composition is homogeneous, and wherein the dosing is until the patient achieves a IGA score of clear (0) or almost clear (1) is reached or the patient has a 2-point improvement in the IGA score. In one embodiment, the active is solubilized in the oil phase of the composition.

In one embodiment, the patient achieves an IGA score of clear (0) or almost clear (1) from baseline. In another embodiment, the patient achieves a 2-point improvement from baseline on the IGA score. As a degree of measurement of severity, the patient will have started with an IGA score of ≥2 at baseline for purposes herein. In another embodiment, the patient will have started with an IGA score of ≥3 at baseline.

In one embodiment, the time the patient maintains without relapse, e.g., post treatment is 2 weeks or greater; 1 month or greater; or 2 months or greater, or 3 months or greater up to 12 months or greater. In one embodiment, the course of treatment is 52 weeks or less in duration of treatment, or is 28 weeks or less in duration of treatment, or is 21 weeks or less in duration of treatment, or is 16 weeks or less in duration of treatment, or is 12 weeks or less, or is 8 weeks or less, or is 4 weeks or less. In one embodiment of the invention the time to relapse is not influenced by the duration of treatment.

In another embodiment, there is a method of improving the % body surface area (BSA) of a person affected with atopic dermatitis (AD), the method comprising administering to said person a topical pharmaceutical emulsion composition comprising an effective amount of 3,5-Dihydroxy-4-isopropyl-trans-stilbene or a pharmaceutically acceptable salt thereof, an oil phase, a water phase, a surfactant, and an antioxidant, wherein the emulsion composition is homogeneous, and wherein the % BSA improvement seen in the patient is from about ≥10-29%, or is >≥than 30-49% or is ≥50-69% or is ≥70-89%, or is 90-100%.

Pruritus is the most frequent symptom of AD and potentially has the greatest effect on quality of life. In another embodiment, there is a method of reducing pruritus in a person with atopic dermatitis (AD) comprising administering to said person a topical pharmaceutical emulsion composition comprising an effective amount of 3,5-Dihydroxy-4-isopropyl-trans-stilbene or a pharmaceutically acceptable salt thereof, an oil phase, a water phase, a surfactant, and an antioxidant, and wherein the emulsion composition is homogeneous, and the time to reduction of pruritus, and/or the % BSA affected with pruritus is reduced from a baseline measurement. In one embodiment, the formulations of the present invention may be compared to formulation 1 or 12.

Sleep disturbance is also considered to be a frequent symptom of AD and significantly impacts the quality of life for the patient and their associated family. In another embodiment there is a method of improving quality of sleep in a person with atopic dermatitis (AD), as measured by standard sleep assessments (such as by MOS-6), comprising administering to said person a topical pharmaceutical emulsion composition comprising an effective amount of 3,5-Dihydroxy-4-isopropyl-trans-stilbene or a pharmaceutically acceptable salt thereof, an oil phase, a water phase, a surfactant, and an antioxidant, wherein the 3,5-Dihydroxy-4-isopropyl-trans-stilbene or pharmaceutically acceptable salt thereof is solubilized in the oil phase of the emulsion composition and/or is homogeneous, and whereby sleep is improved.

Caregivers of subjects with AD have a significantly impacted quality of life. In another embodiment there is a method of improving quality of life for caregivers of a person with atopic dermatitis (AD) such as measured by the WPAI or other suitable endpoints, comprising administering to said person a topical pharmaceutical emulsion composition comprising an effective amount of 3,5-Dihydroxy-4-isopropyl-trans-stilbene or a pharmaceutically acceptable salt thereof, an oil phase, a water phase, a surfactant, and an antioxidant, wherein the 3,5-Dihydroxy-4-isopropyl-trans-stilbene or pharmaceutically acceptable salt thereof is solubilized in the oil phase of the emulsion composition and/or is homogeneous.

School-aged children with AD also experience substantial appearance and emotional distress as the result of the outward appearance of their skin and it significantly impacts the quality of life for the patient and their associated family. In another embodiment there is a method of improving quality emotional function in a school-aged person with atopic dermatitis (AD) as measured by emotional function (PED-QL) comprising administering to said person a topical pharmaceutical emulsion composition comprising an effective amount of 3,5-Dihydroxy-4-isopropyl-trans-stilbene or a pharmaceutically acceptable salt thereof, an oil phase, a water phase, a surfactant, and an antioxidant, wherein the 3,5-Dihydroxy-4-isopropyl-trans-stilbene or pharmaceutically acceptable salt thereof is solubilized in the oil phase of the emulsion composition and/or is homogeneous.

Reduction in overall mental and physical function is also considered to be a frequent symptom of AD and significantly impacts the quality of life for the patient and their associated family. In another embodiment there is a method of improving overall quality of life in a person with atopic dermatitis (AD) as measured by standard mental and physical assessments (such as by SF-36) comprising administering to said person a topical pharmaceutical emulsion composition comprising an effective amount of 3,5-Dihydroxy-4-isopropyl-trans-stilbene or a pharmaceutically acceptable salt thereof, an oil phase, a water phase, a surfactant, and an antioxidant, wherein the 3,5-Dihydroxy-4-isopropyl-trans-stilbene or pharmaceutically acceptable salt thereof is solubilized in the oil phase of the emulsion composition and/or is homogeneous.

One embodiment of the invention is a method of obtaining optimal dosing for the treatment of atopic dermatitis in a patient in need thereof, the method comprising administering to said patient a topical pharmaceutical emulsion composition comprising an effective amount of 3,5-Dihydroxy-4-isopropyl-trans-stilbene or a pharmaceutically acceptable salt thereof, an oil phase, a water phase, a surfactant, and an antioxidant, and wherein the emulsion is homogeneous, and dosing continues until the patient achieves a IGA score of clear or almost clear is reached or a 2 point reduction of IGA score is achieved. In one embodiment, the patient achieves an IGA score of clear or almost clear. In one embodiment, the course of treatment is 28 weeks or less in duration of treatment, or is 21 weeks or less, or is 16 weeks or less in duration of treatment, or is 12 weeks or less in duration of treatment. In one embodiment, the time to relapse post treatment is 3 months or greater. In another embodiment, the time to relapse post treatment is 1 month or greater. In another embodiment, the time to relapse post treatment is 3 months or greater. In another embodiment, the time to relapse post treatment is 6 months or greater. In one embodiment of the invention the time to relapse is not influenced by the duration of treatment.

Another embodiment of the invention is a method for reducing the time to achieving a >50% improvement in IGA scores of clear or almost clear or a 2-point reduction in IGA score in a patient with atopic dermatitis in need thereof, the method comprising administering to said patient a topical pharmaceutical emulsion composition comprising an effective amount of 3,5-Dihydroxy-4-isopropyl-trans-stilbene or a pharmaceutically acceptable salt thereof, an oil phase, a water phase, a surfactant, and an antioxidant, wherein the emulsion composition is homogeneous. A suitable comparator formulation would be formulation 1/1a or 12 (of similar % w/w active). In one embodiment, the time to achievement of the 50% reduction is at 12 weeks. In another embodiment, the time to achievement of the 50% reduction is at 8 weeks.

In one embodiment, the amount of 3,5-Dihydroxy-4-isopropyl-trans-stilbene or a pharmaceutically acceptable salt thereof, administered daily is from 10 mg to 100 mg. In another embodiment, the daily amount administered is from 10 mg to 50 mg.

In a similar manner, psoriasis can be assessed. Of the many psoriasis assessment tools currently available, the Psoriasis Area and Severity Index (PAST) and Physician Global Assessment (PGA) tools are most used. A PASI score is used to measure the severity and extent of psoriasis.

PASI examines four body regions: i) the head and neck, ii) the hands and arms, iii) the chest, abdomen and back (trunk) and iv) the buttocks, thighs and legs. Each region is given a score to show how much of the region is affected by psoriasis (area) and a score to record how bad the psoriasis is (severity). The area score can range from 0 (no psoriasis) to 6 (all of the skin affected). The severity score for each region is reached by adding scores for redness, thickness and scale, each of which is graded from O to 4, giving a maximum of 12. A PASI score of >10 is generally used to indicate a patient with moderate to severe plaque psoriasis.

The PGA scale in its typical use, is a 7-point scale ranging from clear to severe.

| | |
|---|---|
| Severe | Very marked plaque elevation, scaling, |
| | and/or erythema |
| Moderate to Severe | Marked plaque elevation, scaling, and/or |
| | erythema |
| Moderate | Moderate plaque elevation, scaling, and/or |
| | erythema |
| Mild to moderate | Intermediate between moderate and mild |
| Mild | Slight plaque elevation, scaling, and/or |
| | erythema |
| Almost clear | Intermediate between mild and clear |
| Clear | No signs of psoriasis |

One embodiment of the invention is a percent of patients achieving a 50% or a 75% reduction in PASI score (PAST 50 or PASI 75) achieved by using a topical emulsion composition as described herein. This may be a standalone clinical endpoint, or may be used in combination with the patient also reaching a PGA score of 0 or 1 (clear or almost clear) at a defined time point, such as at 8, 12 weeks, 16 weeks, 20, or 24 weeks, or greater than 24 weeks of treatment. As a degree of measurement of severity, the patient will most likely have started with a PGA score of ≥4 at baseline for purposes herein, e.g., one with moderate to severe plaque psoriasis.

One embodiment of the invention is a method of obtaining optimal dosing for the treatment of psoriasis in a patient in need thereof, the method comprising administering to said patient a topical pharmaceutical emulsion composition comprising an effective amount of 3,5-Dihydroxy-4-isopropyl-trans-stilbene or a pharmaceutically acceptable salt thereof, an oil phase, a water phase, a surfactant, and an antioxidant, wherein the emulsion composition is homogeneous, and wherein the dosing continues until the patient achieves a PGA score of clear or almost clear is reached. In one embodiment, the course of treatment is 28 weeks or less in duration of treatment, or is 21 weeks or less, or is 16 weeks or less in duration of treatment, or is 12 weeks or less in duration of treatment. In one embodiment, the time to relapse post treatment is 3 months or greater. In another embodiment, the time to relapse post treatment is 6 months or greater. In one embodiment of the invention the time to relapse is not influenced by the duration of treatment.

Another embodiment of the invention is a method for reducing the time to achieving a >50% improvement in PGA scores of clear or almost clear or a 2-point reduction in PGA score in a patient with psoriasis in need thereof, the method comprising administering to said patient a topical pharmaceutical emulsion composition comprising an effective amount of 3,5-Dihydroxy-4-isopropyl-trans-stilbene or a pharmaceutically acceptable salt thereof, an oil phase, a water phase, a surfactant, and an antioxidant, and wherein the emulsion composition is homogeneous. In one embodiment, the time to achievement of the 50% reduction is at 12 weeks. In another embodiment, the time to achievement of the 50% reduction is at 8 weeks. In one embodiment, the reduction of time may be compared to administration of a suitable comparator such as formulation 1/1a or 12 (of comparable % w/w active).

Alternatively, there is method is achieving a 50% or 75% reduction in PASI score in a patient with psoriasis in need thereof, the method comprising administering to said patient a topical pharmaceutical emulsion composition comprising an effective amount of 3,5-Dihydroxy-4-isopropyl-trans-stilbene or a pharmaceutically acceptable salt thereof, an oil phase, a water phase, a surfactant, and an antioxidant, and wherein the emulsion composition is homogeneous.

In one embodiment, the course of treatment is 52 weeks or less in duration of treatment, or is 28 weeks or less in duration of treatment, or is 21 weeks or less, or is 16 weeks or less in duration of treatment, or is 12 weeks or less in duration of treatment, or is 2 months or less. In one embodiment, the time the patient maintains without relapse, e.g., post treatment (also stated as time to relapse) is 2 weeks or greater, or 1 month or greater, or 2 months or greater or is 3 months or greater up to 12 months or greater. In another embodiment, the time to relapse post treatment is 6 months or greater. In one embodiment of the invention the time to relapse is not influenced by the duration of treatment. One suitable comparator formulation would be formulation 1 or 1a. In one embodiment, the time to achievement of the 50% reduction is at 12 weeks. In another embodiment, the time to achievement of the 50% reduction is at 8 weeks.

In another embodiment there is a method for reducing the time to achieving a >50% improvement in PGA scores of clear or almost clear or a 2-point reduction in PGA score and a PASI 50 or PASI 75 reduction in a patient with psoriasis in need thereof, comprising administering to said patient a topical pharmaceutical emulsion composition comprising an effective amount of 3,5-Dihydroxy-4-isopropyl-trans-stilbene or a pharmaceutically acceptable salt thereof, an oil phase, a water phase, a surfactant, and an antioxidant, and wherein the emulsion composition is homogeneous. In one embodiment, the PASI score is a PASI 75. In another embodiment, the patient achieves a PGA score of clear or almost clear.

In another embodiment, there is a method of improving the time to reaching a PASI 50 or PASI 75 score in a person affected in a person with psoriasis, the method comprising administering to said person a topical pharmaceutical emulsion composition comprising an effective amount of 3,5-Dihydroxy-4-isopropyl-trans-stilbene or a pharmaceutically acceptable salt thereof, an oil phase, a water phase, a surfactant, and an antioxidant, and wherein emulsion composition is homogeneous. In one embodiment, the time to reach a PASI 50 is at 16 weeks, or 12 weeks, or at 8 weeks. In another embodiment, the time to reach a PASI 70 is at 16 weeks, or 12 weeks, or at 8 weeks. A suitable comparator formulation would be formulation 1/1a or 12 (of comparable % w/w active).

Reduction in overall mental and physical function is also considered to be a frequent symptom of PSO and significantly impacts the quality of life for the patient and their associated family. In another embodiment there is a method of improving overall quality of life in a person with atopic dermatitis (PSO) as measured by standard mental and physical assessments (such as by SF-36) comprising administering to said person a topical pharmaceutical emulsion composition comprising an effective amount of 3,5-Dihydroxy isopropyl-trans-stilbene or a pharmaceutically acceptable salt thereof, an oil phase, a water phase, a surfactant, and an antioxidant, wherein the 3,5-Dihydroxy-4-isopropyl-trans-stilbene or pharmaceutically acceptable salt thereof is solubilized in the oil phase of the emulsion composition and/or is homogeneous.

Caregivers of subjects with AD have a significantly impacted quality of life. In another embodiment there is a method of improving quality of life for caregivers of a person with atopic dermatitis (AD) such as measured by WPAI scoring, comprising administering to said person a topical pharmaceutical emulsion composition comprising an effective amount of 3,5-Dihydroxy-4-isopropyl-trans-stilbene or a pharmaceutically acceptable salt thereof, an oil phase, a water phase, a surfactant, and an antioxidant, wherein the 3,5-Dihydroxy-4-isopropyl-trans-stilbene or pharmaceutically acceptable salt thereof is solubilized in the oil phase of the emulsion composition and/or is homogeneous.

According to an embodiment, the invention provides a method of treating a dermatological condition or disorder in a patient in need thereof, the method comprising administering to said patient a topical pharmaceutical emulsion composition comprising an effective amount of 3,5-Dihydroxy-4-isopropyl-trans-stilbene or a pharmaceutically acceptable salt thereof, an oil phase, a water phase, a surfactant, and an antioxidant, and wherein the emulsion composition is homogeneous. In another embodiment, the 3,5-Dihydroxy-4-isopropyl-trans-stilbene or pharmaceutically acceptable salt thereof is solubilized in the oil phase of the emulsion composition. In another embodiment, the oil phase comprises mineral oil or petrolatum. In another embodiment, if the oil phase comprises mineral oil or petrolatum, then a second oil phase component is present, and if the oil phase contains both mineral oil and petrolatum then a third oil phase component is present.

Another aspect of the invention is the use of a topical pharmaceutical emulsion composition comprising an effective amount of the active ingredient 3,5-Dihydroxy-4-isopropyl-trans-stilbene or a pharmaceutically acceptable salt thereof, an oil phase, a water phase, a surfactant, and an antioxidant, and wherein the emulsion composition is homogeneous, in the manufacture of a medicament for the treatment of inflammatory skin diseases or disorders in a patient. In one embodiment, the active ingredient is solubilized in the oil phase of the emulsion composition. In another embodiment, the oil phase comprises mineral oil or petrolatum. In another embodiment, if the oil phase comprises mineral oil or petrolatum, then a second oil phase component is present, and if the oil phase contains both mineral oil and petrolatum then a third oil phase component is present. In one embodiment, the disease or disorder is atopic dermatitis, psoriasis or acne.

The compositions of the present invention may be used in a veterinary setting or in a medical setting, topically. It is recognized that the patient or subject may be an animal, a domestic animal, such as a mammal, including horses, cows, pigs, sheep, poultry, fish, cats, dogs and zoo animals. In one embodiment, the patient is an animal. In another embodiment, the patient is a mammal. In another embodiment, the mammal is a human. In another embodiment, the human is an adult, or a pediatric patient. In one embodiment, the pediatric patient is a child. In another embodiment, the pediatric patient is 3 months to 2 years of age and older.

In one embodiment, the dermatological condition or disorder for which treatment is sought is an inflammatory skin disease (e.g., a chronic inflammatory skin disease such as dermatitis (e.g., atopic dermatitis, contact dermatitis, eczematous dermatitis, or seborrhoic dermatitis), acne, psoriasis, rosacea, or aging skin.

In some aspects, the dermatological condition or disorder is selected from the group for the treatment of a skin disease, wherein the skin disease comprises a skin disorder of persistent inflammation, cell kinetics, and differentiation (e.g., psoriasis, psoriatic arthritis, exfoliative dermatitis, Pityriasis rosea, Lichen planus, Lichen nitidus, or porokeratosis); a skin disorder of epidermal cohesion, vesicular and bullous disorders (e.g., pemphigus, bulluous pemphigoi, epidermamolysis bullosa acquisita, or pustular eruptions of the palms or soles); a skin disorder of epidermal appendages and related disorders (e.g., hair disorders, nails, rosacea, perioral dermatitis, or follicular syndromes); a skin disorder such as an epidermal and appendageal tumors (e.g., squamous cell carcinoma, basal cell carcinoma, keratoacanthoma, benign epithelial tumors, or merkel cell carcinoma); a disorder of melanocytes (e.g., pigmentary disorders, albinism, hypomelanoses and hypermelanoses, melanocytic nevi, or melanoma); a skin disorder of inflammatory and neoplastic disorders of the dermis (e.g., erythema elavatum diutinum, eosinophils, granuloma facilae, pyoderma gangrenosum, malignant atrophic papulosis, fibrous lesions of dermis and soft tissue, or Kaposi sarcoma); a disorder of the subcutaneous tissue (e.g., panninculitis or lipodystrophy); a skin disorder involving cutaneous changes of altered reactivity (e.g., urticaria, angiodererma, graft-vs-host, allergic contact dermatitis, autosensitization dermatitis, atopic dermatitis, or seborrheic dermatitis); a skin change due to mechanical and physical factors (e.g., thermal injury, radiation dermatitis, corns, or calluses); photodamage (e.g., acute and chronic UV radiation, or photosensitization); or a skin disorder due to microbial agents (e.g., leprosy, lyme borreliosis, onychomycosis, tinea pedra, rubella, measles, herpes simplex, EBY (Epstein-Barr virus), HPV (Human papillomavirus) (e.g., HPV6 & 7), warts, or prions).

In one embodiment, the inflammatory disorder is selected from the group consisting of psoriasis, and atopic dermatitis and acne. In an embodiment, the dermatological condition or disorder is psoriasis. In another embodiment, the dermatological condition or disorder is atopic dermatitis. In another embodiment, the dermatological condition or disorder is acne.

### Definitions

The phrase “therapeutically effective amount” or “effective amount” is used herein to refer to an amount of the active ingredient sufficient to have a therapeutic effect upon administration, e.g., that amount which will cause an improvement or change in the condition for which it is applied when applied to the affected area repeatedly over a period chime. Effective amounts will vary with the particular condition being treated, the severity of the condition, the duration of the treatment, the stage of advancement of the condition, the body surface area affected with the clinical condition, and the specific components of the composition. An effective amount of the active ingredient for treatment of a condition or disorder can be determined by standard clinical techniques. Appropriate amounts in any given instance will be readily apparent to those skilled in the art or capable of determination by routine experimentation. The compositions are generally applied in topical manner to the affected area, i.e., localized application to the skin region where the clinical abnormality is manifest.

Concentrations, amounts, solubilities, and other numerical data may be presented herein in a range format. It is to be understood that such range format is used merely for convenience and brevity and should be interpreted flexibly to include not only the numerical values explicitly recited as the limit of the range, but also to include all the individual numerical values or subranges encompassed within that range as if each numerical value and sub-range is explicitly recited. All numbers expressing quantities, percentages or proportions, and other numerical values used in the specification, are to be understood as being modified in all instances by the term “about”.

For example, a concentration range of 0.1 to 5 ng/ml should be interpreted to include not only the explicitly recited concentration limits of 0.1 ng/ml and 5 ng/ml but also to include individual concentrations such as 0.2 ng/ml, 0.8 ng/ml, 1.0 ng/ml 2.2 ng/ml, 3.6 ng/ml, and sub-ranges such as 0.3-2.5 ng/ml, 1.8-3.2 ng/ml, etc. This interpretation should apply regardless of the breadth of the range or the characteristic being described.

The terms “administering” and “administration” are used herein to mean any method which in sound medical practice delivers the pharmaceutical emulsion composition to a patient in such a manner as to provide the desired therapeutic effect.

As used herein, “topical” administration of the pharmaceutical emulsion composition refers to application to and diffusion through the stratum corneum, including application to psoriatic lesions and broken skin.

As used herein, in the in vitro skin penetration studies the term “epidermis” includes the stratum corneum and tissue or layers down to the basement membrane, as isolated by heat separation treatment.

As used herein, in the in vitro skin penetration studies with ex-vivo human abdominal skin dermatomed at a thickness of 500 microns(+/−100 microns); the terms “epidermis” is the top/superficial layer obtained by heat separation procedure, and the term “dermis” is the underlying layer (after a washing/tape striping procedure).

The terms “treatment” or “treating” of a dermatological condition or disorder encompasses alleviation of at least one symptom thereof, a reduction in the severity thereof, or the delay, prevention or inhibition of the progression thereof. Treatment need not mean that the condition or disorder is totally cured. A useful pharmaceutical emulsion composition herein need only to reduce the severity of the condition or disorder, reduce the severity of symptoms associated therewith, provide improvement to a patient's quality of life, or delay, prevent or inhibit the onset of the condition or disorder. A treatment need not be effective in every member of a population, e.g., a population of patients with atopic dermatitis, to have clinical utility, as is recognized in the medical and pharmaceutical arts.

The term “pharmaceutically acceptable salt thereof” refers to salts that are safe and effective for topical use in the patient and possess the desired pharmaceutical activity. Such salts include salts formed when an acidic proton is replaced with a metal ion (e.g., alkali metal ion, alkaline earth metal ion, or aluminum ion).

The terms “pharmaceutically acceptable” and “dermatologically acceptable” mean approvable by a regulatory agency or listed in a Pharmacopeia or other generally recognized guide for use in animals, and more particularly in humans.

As used herein, the term “skin penetration” refers to the diffusion of the 3,5-Dihydroxy-4-isopropyl-trans-stilbene or a pharmaceutically acceptable salt thereof through the stratum corneum and into the epidermis and/or dermis of the skin.

As used herein, “patients” includes human patients, including adult, teens and children (e.g., pediatric patients). A pediatric patient can include teenagers under the age of 18. A child for purposes herein is under the age of 12.

As used herein, “solubilize” means dissolved in a particular phase in an amount ≥50% w/w, or ≥60% w/w, or ≥70% w/w, or ≥80% w/w, or ≥90% w/w or ≥95% w/w, based on the percent by weight of the final composition prepared.

As used herein, “homogeneous” means a uniform dispersal of one phase within the other. In the instance of an o/w emulsion it is the uniform dispersal of the oil phase within the water phase. A homogeneous emulsion is an emulsion or mixture having continuous, uniform droplet size. Preferably, this is visually observed using microscopy. As described above, the homogeneous emulsions described herein have droplet sizes that meet the D90 and D50 values provided. In addition, the term “homogeneous” can be described in at least two ways, “range” and “span,” which describes how close the size of all of the droplets are to each other. The “range” of droplet size is calculated as the difference between D10 and D90: (D90−D10). For example, the “range” of the emulsions can be about 0.6 μm, if the droplet sizes range from 0.1 μm (D10) to about 0.7 μm (D90)=0.7 μm−0.1 μm. In some embodiments described herein, a homogeneous emulsion has a range of about 5 μm. The droplet sizes in the homogeneous emulsions described herein have a tight range of sizes. The “span” of droplet size is calculated as: (D90−D10)/D50. The “span” of the emulsions (which is a unit-less number) can be about 3 if, for example, D50 is about 0.2 μm, D90 is about 0.7 μm and D10 is about 0.1 μm=(0.7−0.1)/0.2. In some embodiments described herein, a homogeneous emulsion has a span of about 2 to about 3.

Further, the homogeneity of the active ingredient throughout each prepared formulation described herein demonstrates uniformity throughout each batch, for example, the active ingredient is at a concentration as described on the label with a range of about 90.0% to about 110.0% of the label claim with a relative standard deviation of not more than about 5%. For example, formulations containing 1% active ingredient will have homogeneity throughout the batch which will not be greater than 0.90% to 1.10%. These measurements are made within each container or tube, at, for example, the top, middle and bottom of a containers.

Any concentration range, percentage range or ratio range recited herein is to be understood to include concentrations, percentages or ratios of any integer within that range and fractions thereof, such as one tenth and one hundredth of an integer, unless otherwise indicated.

Unless otherwise indicated, all percentages are based on the percent by weight of the final composition prepared, and all totals equal “100% by weight.

It should be understood that the terms “a” and “an” as used herein refer to “one or more” of the recited components. It will be clear to one of ordinary skill in the art that the use of the singular includes the plural unless specifically stated otherwise.

Throughout the application, descriptions of various embodiments use “comprising” language, however in some specific instances, an embodiment can alternatively be described using the language “consisting essentially of” or “consisting of”.

“Substantially free” of a specified component refers to a composition with less than about 1% by weight of the specified component. “Free” of a specified component refers to a composition where the specified component is absent.

As the biological profile, or the pK/pD of 3,5-Dihydroxy-4-isopropyl-trans-stilbene or a pharmaceutically acceptable salt, depends on the absence of degradation products and delivery of the active ingredient to the appropriate layers of the skin in efficacious amounts, compositions such as described herein offer patients a novel therapeutic treatment option for various inflammatory skin conditions.

Other terms used herein are intended to be defined by their well-known meanings in the art. The examples set forth below are illustrative of the present invention and are not intended to limit, in any way, the scope of the present invention. The following examples illustrate the invention. These examples are not intended to limit the scope of the present invention, but rather to provide guidance to the skilled artisan to prepare and use the compounds, compositions, and methods of the present invention.

While particular embodiments of the present invention are described, the skilled artisan will appreciate that various changes and modifications can be made without departing from the spirit and scope of the invention.

### EXAMPLES

### Example 1—Cream Compositions Comprising 3,5-Dihydroxy-4-isopropyl-trans-stilbene and Chemical and Physical Stability

TABLE 1
Cream Compositions studied

| | Formulation Number | | | | | | | |
|---|---|---|---|---|---|---|---|---|
| | | 10 | 12 | | | | | |
| | 1 | (placebo) | (tergus) | 17 | 21 | 22 | 23 | 24 |
| Ingredient | % w/w | % w/w | % w/w | % w/w | % w/w | % w/w | % w/w | % w/w |
| Active Phase | | | | | | | | |
| Active ingredient | 0.50 | 0.00 | 0.50 | 0.50 | 1.00 | 2.00 | 2.00 | 4.00 |
| Propylene glycol | 10.00 | 10.00 | 10.00 | 10.00 | 10.00 | 10.00 | 10.00 | 10.00 |
| Diethylene glycol | 2.00 | 2.00 | 2.00 | 2.00 | 2.00 | 2.00 | 2.00 | 2.00 |
| monoethyl ether | | | | | | | | |
| Polysorbate 80 | 1.00 | 1.50 | 1.50 | 1.50 | 1.50 | 1.50 | 2.10 | 2.10 |
| Water Phase | | | | | | | | |
| Purified water | 53.09 | 53.18 | 52.68 | 65.18 | 64.68 | 63.68 | 63.18 | 61.18 |
| Sodium acetate | 0.51 | — | — | — | — | — | — | — |
| Glacial acetic acid | 0.10 | — | — | — | — | — | — | — |
| Sodium citrate | — | 0.19 | 0.19 | 0.19 | 0.19 | 0.19 | 0.19 | 0.19 |
| Citric acid | — | 0.08 | 0.08 | 0.08 | 0.08 | 0.08 | 0.08 | 0.08 |
| Disodium EDTA | — | 0.10 | 0.10 | 0.10 | 0.10 | 0.10 | 0.10 | 0.10 |
| Methylparaben | 0.18 | — | — | — | — | — | — | — |
| Propylparaben | 0.02 | — | — | — | — | — | — | — |
| Oil phase | | | | | | | | |
| Emulsifying Wax, BP | 7.20 | — | — | — | — | — | — | — |
| POLAWAX (non-ionic | — | 7.20 | 7.20 | 7.20 | 7.20 | 7.20 | — | — |
| emulsifying wax)/ | | | | | | | | |
| Emulsifying Wax, NF | | | | | | | | |
| Cetostearyl alcohol | — | — | — | — | — | — | 5.90 | 5.90 |
| White petrolatum | 17.00 | 16.50 | 16.50 | — | — | — | — | — |
| Mineral oil | 6.00 | 6.00 | 6.00 | — | — | — | — | — |
| MCT | — | — | — | 10.00 | 10.00 | 10.00 | 10.00 | 10.00 |
| BHT | — | 0.10 | 0.10 | 0.10 | 0.10 | 0.10 | 0.10 | 0.10 |
| Benzoic acid | — | 0.25 | 0.25 | 0.25 | 0.25 | 0.25 | 0.25 | 0.25 |
| Steareth 2 | 1.50 | 1.80 | 1.80 | 1.80 | 1.80 | 1.80 | 2.50 | 2.50 |
| Steareth 20 | 0.90 | 1.10 | 1.10 | 1.10 | 1.10 | 1.10 | 1.60 | 1.60 |
| | 100.00 | 100.00 | 100.00 | 100.00 | 100.00 | 100.00 | 100.00 | 100.00 |

The samples were also subjected to chemical stability analysis by HPLC using the following conditions: Zorbax Bonus reverse phase column: 150×4.6 mm, 3.5 μm particle size with 4.0 mm guard frit, Column temp: 25° C., Autosampler temp: ambient, Flow rate: 1.0 ml/min, Injection volume: 15 μl, Detection: 235 nm, Run time: 50 minutes, Mobile Phase A: 0.1% TFA in water, Mobile Phase B: Acetonitrile.

TABLE 2
HPLC elution gradient

| Time (minutes) | % B |
|---|---|
| 0 | 5 |
| 40 | 100 |
| 45 | 100 |
| 45.1 | 5 |
| 50 | 5 |

Formulations 1 and 12 it was observed from microscopic analysis that the emulsion structure of these formulations was non-uniform and had wax-like material present. The non-uniform emulsion is illustrated in FIG. 1A and was determined to be dependent on the concentration of the active ingredient. In other words, there was no such observation in Formulation 10 (placebo) and increased levels of heterogeneity were observed with increasing levels of active ingredient.

To identify which semi-solid ingredient is responsible for forming the non-uniform emulsion, experiments were conducted where each semi-solid ingredient was systematically substituted as follows:

TABLE 3

| Remove | Substitute |
|---|---|
| Emulsifying wax NF | Water |
| Steareth-2 & Steareth-20 | Water |
| White petrolatum | Mineral oil |
| White petrolatum | Water |
| Emulsifying wax NF & white petrolatum | Water |

The formulation that was free of petrolatum was observed to have a uniform emulsion. Additional characterization was conducted using hot stage optical microscopy, XRD, IR microscopy and fluorescent microscopy. These techniques confirmed that the wax-like material was comprised of petrolatum and was a function of the concentration of the active ingredient present. These observations suggested that the petrolatum was not adequately emulsified and that the active ingredient was not solubilized in either the water phase or the oil phase of the emulsion.

Formulation 17 were subjected to microscopic analysis and was found to be homogeneous and the dispersed oil phase had a small particle size and a uniform emulsion was observed. The difference in the appearance of a formulation with 10% MCT as the oil phase versus a formulation with larger amounts of white petrolatum and mineral oil as the oil phase is illustrated in FIG. 1B. Without wishing to be bound by theory, it is thought that because the active ingredient is amphiphilic it has the potential to destabilize the emulsion, particularly when appropriate solvent selection is not made. It is thought that by using the appropriate solvent for the active ingredient in the oil phase, a more stable emulsion may be obtained.

Formulation 17 (0.5% active ingredient) was prepared, along with equivalent formulations containing 1% active ingredient, 2% active ingredient (Formulations 21 and 22, respectively). Alternative formulations where 7.2% POLAWAX was replaced with 5.90% cetostearyl alcohol and increased surfactant levels were also prepared (Formulations 23 and 24). Formulation 17 met all chemical and physical stability specifications when stored at standard ICH conditions for up to 6 months. Thus, one embodiment of the invention is a chemically and physically stable pharmaceutical emulsion composition when stored at standard ICH conditions for up to 6 months comprising the active ingredient 3,5-Dihydroxy-4-isopropyl-trans-stilbene or a pharmaceutically acceptable salt thereof, an oil phase, a water phase, a surfactant, and an antioxidant, and wherein the active is solubilized in the oil phase of the emulsion composition. Another embodiment of the invention is a chemically and physically stable pharmaceutical emulsion composition when stored at standard ICH conditions for up to 6 months comprising the active ingredient 3,5-Dihydroxy-4-isopropyl-trans-stilbene or a pharmaceutically acceptable salt thereof, an oil phase, a water phase, a surfactant, and an antioxidant, and wherein the emulsion is homogeneous. In another embodiment, the active is solubilized in the oil phase of the emulsion composition. In another embodiment, the average droplet size of the discontinuous phase is about 0.1 micron to about 1 micron.

### Example 2—Method of Preparation of Final Formulations

The cream compositions described in Example 1 were prepared using the following general method:

### Oil Phase:

1. To a suitably sized vessel, add oil phase ingredients (e.g., MCT, POLAWAX NF, benzoic acid, BHT, propylene glycol, diethylene glycol monoethyl ether, polysorbate 80, steareth 2, steareth 20 and the active ingredient), initiate mixing and heat to 70-80° C., continue mixing until the active ingredient is dissolved and the phase is uniform in appearance.

### Water Phase:

2. Add water phase ingredients to the main mixing vessel (e.g., water and sodium citrate, citric acid and Edetate disodium). Begin mixing and heat to 70-80° C. Mix until all materials are fully dissolved and the water phase is uniform in appearance.

### Emulsification:

3. Once the water and oil phases are free of undissolved particles, uniform in appearance and at 70-80° C., slowly vacuum transfer the oil phase into the main vessel containing the water phase. Scrape down the oil phase vessel and transfer to the main vessel.
4. Once the transfer and scrape-down are complete, maintain mixer settings and mix for 5-10 minutes while maintaining product temperature between 70-80° C. Verify the product is uniform in appearance.
5. Transfer batch to holding vessel. Cool.

Thus, another embodiment of the present invention is a method of making an emulsion composition comprising
i) mixing and heating the oil phase ingredients and the active ingredient 3,5-Dihydroxy-4-isopropyl-trans-stilbene or a pharmaceutically acceptable salt thereof until dissolved;
ii) mixing the water phase ingredients until fully dissolved;

adding the oil phase ingredient of step (i) and the water phase ingredients of step (ii) and mixing until uniform in appearance. In one embodiment, the resulting emulsion composition is homogeneous. In another embodiment, the active ingredient is solubilized in the oil phase. In another embodiment, the average droplet size of the discontinuous phase is about 0.1 micron to about 35 microns. In another embodiment, the average droplet size of the discontinuous phase is about 0.1 micron to about 25 microns. In another embodiment, the average droplet size of the discontinuous phase is about 0.1 micron to about 15 microns. In another embodiment, the average droplet size of the discontinuous phase is about 0.1 micron to about 10 microns. In another embodiment, the average droplet size of the discontinuous phase is about 0.1 micron to about 5 microns. In another embodiment, the average droplet size of the discontinuous phase is about 0.1 micron to about 1 micron. In another embodiment, the average droplet size of the discontinuous phase is about 0.5 microns. In another embodiment, the average droplet size of the discontinuous phase is from about 0.1 to about 0.75 microns. In another embodiment, the average droplet size of the discontinuous phase (D50) is about 0.1 micron to 5 microns. In another embodiment, the average droplet size of the discontinuous phase (D50) is about 0.1 micron to 1 micron.

In another embodiment, there is a method of making an emulsion composition comprising
i) mixing and heating the oil phase ingredients and the active ingredient 3,5-Dihydroxy-4-isopropyl-trans-stilbene or a pharmaceutically acceptable salt thereof until dissolved;
ii) mixing the water phase ingredients until fully dissolved;

adding the oil phase ingredient of step (i) and the water phase ingredients of step (ii) and mixing until uniform in appearance, and the emulsion is homogeneous and optionally has an average droplet size of the oil phase/discontinuous phase (D50) of about 0.1 micron to 5 microns.

### Example 3—Effects of the Cream Compositions Comprising 3,5-Dihydroxy-4-isopropyl-trans-stilbene on Skin Penetration Properties

The object of this study was to determine the in-vitro skin penetration of 3,5-Dihydroxy-4-isopropyl-trans-stilbene.

In particular, Formulations 17 and 21-24 were tested for their ability to deliver the active ingredient into the epidermis and dermis, and were compared against Formulations 1 and 12.

Briefly, Formulations 1, 12, 17 and 21-24 comprising 3,5-Dihydroxy-4-isopropyl-trans-stilbene were evaluated using a skin penetration assay. This study was designed to determine drug permeation into the epidermis, dermis and receiving fluid. Freshly excised human abdominal skin was dermatomed to a thickness of 500±100 μm and mounted on flow-through diffusion cells using donor blocks to provide a leak proof seal, exposing a surface area of 1.0 cm². Diffusion cells were connected to multi-channel pumps with a flow rate of approximately 0.6 mL/hr with PBS. Each cell was then equilibrated in a heating manifold to ensure a skin surface temperature of 32° C. (for at least 30 min prior to dosing). Test articles were applied at a dose of 10 μl per skin section (10 mg test article/cm²). Test articles were applied to two separate donors to capture inter-individual variation and to at least seven skin sections per donor to capture intra-individual variation. At 15 hours, post-application, the skin surface was wiped with cotton swabs, and tape-stripped three times to remove any residual test article. The washed skin was heat split at the epidermal and dermal junction. The skin layers were placed in separate homogenization vials and the drug was extracted. The extraction of active ingredient from separate test articles served to assess the efficiency of the extraction solvent. The recovered drug concentrations were used to calculate skin penetration into the epidermis and dermis as a percentage of applied doses. 3,5-Dihydroxy-4-isopropyl-trans-stilbene was detected using a liquid chromatography/mass spectrometry.

The amount of 3,5-Dihydroxy-4-isopropyl-trans-stilbene delivered into the epidermis, dermis (15 hours post application) from Formulations 1, 12, 17 and 21-24 is shown in FIG. 2.

Error bars represent the standard error of the mean (SEM) of 8 to 14 replicates per formulation (four skin donors). Note: Since the dosing area of the diffusion cell was 1 cm², the amounts of 3,5-Dihydroxy-4-isopropyl-trans-stilbene illustrated in FIG. 2 represent μg/cm².

When comparing the 0.5% w/w 3,5-Dihydroxy-4-isopropyl-trans-stilbene formulations, it was observed the amount of 3,5-Dihydroxy-4-isopropyl-trans-stilbene delivered into the epidermis were similar for Formulation 17, Formulation 1 and Formulation 12. However, Formulation 17 delivered approximately 3-fold more 3,5-Dihydroxy isopropyl-trans-stilbene to the dermis compared to Formulation 12 and Formulation 1. In addition, it was observed from the data for Formulations 17, 21 and 22 that the active ingredient was delivered in a dose dependent manner. Similarly, it was observed from the data for Formulations 23 and 24 that the active ingredient was delivered in a dose dependent manner.

There appeared to be an inverse relationship in the amount of 3,5-Dihydroxy-4-isopropyl-trans-stilbene delivered into the receiving fluid over fifteen hours, where Formulation 17 showed a 2-4-fold lower amount compared to Formulation 12 and Formulation 1. Similar to the amounts delivered to the skin layers (epidermis and dermis), it was observed from the data for Formulations 17, 21 and 22 that the active ingredient was delivered in a dose dependent manner into the receiving fluid. Similarly, it was observed from the data for Formulations 23 and 24 that the active ingredient was delivered in a dose dependent manner into the receiving fluid.

### Example 4—Effects of the Cream Compositions Comprising 3,5-Dihydroxy-4-isopropyl-trans-stilbene on Skin Deposition

Previous studies, showed Formulation 17 delivered higher amounts of 3,5-Dihydroxy-4-isopropyl-trans-stilbene to the dermis with lower amounts partitioning into the receiving fluid compared to Formulation 12. To further explore this observation, a study was conducted in human ex vivo skin to measure dermal levels every three hours (0-15 hours) and at 24 hours and compared to the concentrations measured in the receiving fluid.

Formulations 12 and 21 comprising 0.5% and 1.0% 3,5-Dihydroxy-4-isopropyl-trans-stilbene respectively were evaluated using an ex vivo human skin penetration assay. This study was designed to determine drug permeation into the epidermis, dermis and receiving fluid with more additional sampling for skin deposition. Freshly excised human abdominal skin was dermatomed to a thickness of 500±100 μm and mounted on flow-through diffusion cells using donor blocks to provide a leak proof seal, exposing a surface area of 1.0 cm². Diffusion cells were connected to multi-channel pumps with a flow rate of approximately 0.6 ml/hr with PBS. In order to keep the skin integrity and prevent bacterial growth, the seventy-two hour study receiving fluid contained 1% antibiotic-antimycotic. Each cell was then equilibrated in a heating manifold to ensure a skin surface temperature of 32° C. (for at least 30 min prior to dosing). Test articles were applied at a dose of 10 μl per skin section (10 mg test article/cm²). Test articles were applied to two separate donors to capture inter-individual variation and to at least seven skin sections per donor to capture intra-individual variation. Receiving fluid was collected hourly for 72 hours as a measurement of active ingredient penetrating through the skin. At 3, 6, 9, 12, and 15 hours post-application, the skin surface was wiped with a cotton swab and tape-stripped three times to remove any residual test article considered not penetrating the skin. The washed skin was heat split at the epidermal and dermal junction. The skin layers were placed in separate homogenization vials and the drug was extracted using an Omni bead homogenizer. The recovered drug concentrations were used to calculate skin penetration into the epidermis and dermis as a percentage of applied doses. 3,5-Dihydroxy-4-isopropyl-trans-stilbene was detected using a liquid chromatography/mass spectrometry method described previously.

Formulation 21 showed the same trend observed previously, where the concentrations of 3,5-Dihydroxy-4-isopropyl-trans-stilbene delivered to the dermis were higher compared to Formulation 12. See FIG. 4. The trend for increased dermal deposition or tissue residency from Formulation 21 appeared as early as 3 hours post application and continued over the 15 hours of dosing.

Later time points (i.e., greater than 15 hours) were not considered for tissue analysis (epidermis and dermis) as the skin started losing its integrity, which could potentially result in inaccurate measured levels of active ingredient.

The same inverse relationship in the amount of 3,5-Dihydroxy-4-isopropyl-trans-stilbene delivered into the receiving fluid was observed, where Formulation 21 showed a 1.4 fold lower amount compared to Formulation 12 (FIG. 5). This trend for lowered delivery to the receiving fluid was consistent over the entire 72 hours of dosing as both formulations appeared to be in a steady state after 12 hours (lag phase). Despite Formulation 21 (1% w/w) having double the concentration of active ingredient as Formulation 12 (0.5% w/w), Formulation 21 was not able to deliver equivalent amounts into the receiving fluid. This suggests Formulation 21 is able to alter tissue residence preventing the active ingredient from partitioning from the skin into the receiving fluid.

In one embodiment of the invention is the use of a formulation such as described above which can provide lower systemic exposure of the active ingredient to the patient during use. In another embodiment, the dosing frequency to the affected area(s) may be now be dosed less frequently than previously envisioned. Application of a composition of the present invention may be applied to affected areas twice daily, once daily, once every other day; twice weekly; three times weekly, or once weekly, with the dose represented by any of the embodiments herein. In another embodiment, the treatment may be administered in two phases, an initial dosage frequency such as once or twice daily, followed by a maintenance phase, such as every other day; twice weekly; three times weekly, or once weekly.

### Example 5—Biological Activity in Ex Vivo Human Skin from Cream Compositions Comprising 3,5-Dihydroxy-4-isopropyl-trans-stilbene

Target profiling revealed 3,5-Dihydroxy-4-isopropyl-trans-stilbene to be an activator of the AhR pathway in intestinal human colon adenocarcinoma. BioMAP®, a system that can offer physiologically relevant insights about a compound prior to lengthy and expensive animal or clinical studies, confirmed that Ahr is likely to be a primary target of 3,5-Dihydroxy-4-isopropyl-trans-stilbene. 3,5-Dihydroxy-4-isopropyl-trans-stilbene has also been shown to induce the expression of the AhR target gene, cyp1a1, in primary human keratinocytes, and human or rodent leukocytes. A model using freshly excised human skin has been developed to explore intrinsic efficacy of active ingredients (i.e., target engagement). This model allows topical formulations to be applied to explore pharmacodynamic activity in the skin. This study compares the biological activity of 3,5-Dihydroxy-4-isopropyl-trans-stilbene through Cyp1A1 induction from Formulations 17, 21 and 22 as compared to Formulation 12.

Tissue culture using Static Cells: Freshly excised healthy human skin was dermatomed to 750 um and cleaned with antibiotic/antimycotic solution made up as 1% GIBCO™ Antibiotic-Antimycotic (100×), 0.1% Gentamicin in 1× Dulbecco's Phosphate Buffered Saline. Twelve mm diameter biopsies were cut using disposable single-use biopsy punches and washed in antibiotic/antimycotic solution for 5-10 minutes. Skin biopsies were placed on autoclaved 7 mm (0.38 cm²) unjacketed static cells with 2 ml receptor volume and leak proof seal was maintained using metal clamps and donor chamber. Receptor chamber was filled with cornification media using pasteur pipette to dispense in sampling port. Static cells were then placed in a humidified incubator at 37° C. Test articles were applied topically (to the exposed/dry epidermis) on Day 1. (Day−1). Twenty-four hours later (Day 0), media in the receptor chamber was replaced with an activation cocktail designed to stimulate skin-resident immunocompetent cells. Twenty-four hours later (Day 1) the tissue was removed, minced to less than 1×1×1 mm pieces and stored in 10× volume of RNA later with 300 μl of RNeasy Lysis Buffer supplemented with 1% 2-BetaMercapto-Ethanol for RNA isolation.

Cornification media: Media consisted of 237 ml of Dulbecco's Modified Eagle Medium (DMEM), 237 ml Ham's F-12K Medium, 1 ml 90 mM Adenine, 1 mL 0.94M CaCl, 1 ml 10 nM Tri-iodothyronine, 1 ml Insulin-Transferrin-Selenium-Ethanolamine (ITS-X) (100×), 5 ml Antibiotic-Antimycotic (100×), 10 ml Fetal Bovine Serum (FBS), 5 ml GlutaMAX™ Supplement, 0.1 ml 50 mg/ml Gentamicin.

Activation Cocktail: An ex vivo human skin target engagement model was originally developed to mimic the pro-inflammatory state of lesional psoriatic skin. The model may be found in Smith et al., PLOS ONE, DOI: 10.1371/journal.pone.0147979; Feb. 12, 2016 and is referenced herein as the sRICA (skin-resident immune cell activation) assay. As such, skin resident immunocompetent cells were activated in situ with a combination of 1 μg/ml purified NA/LE Mouse anti-human CD3, 2 μg/ml CD28, 1 μg/ml anti-human IFN-gamma, 1 μg/ml antihuman IL-4 antibodies and 10 ng/ml recombinant human (rh) IL-1b/IL-1F2, 10 ng/ml rh IL-6 (R&DSystems, 1 ng/ml rh TGF-b 1, and rh IL-21. All components incorporated in a single mixture with the cornification media (i.e., activation cocktail).

RNA Isolation & Quantitation: Approximately 40 mg of minced tissue was added to homogenization tubes containing 2.8 and 1.4 mm ceramic beads. The tissue was disrupted using high-throughput bead mill homogenizer machine at 6300 rpm for 30 seconds and 10 cycles with a 2-minute ice break. The homogenate was digested by adding 490 μl of water containing 10 ul Proteinase Kat 55° C. for 15 minutes. Digested tissue was spun down for 3 minutes at 10,000×g to pellet cell debris and the supernatant was used for RNA isolation using Qiagen's Mini RNA Isolation kit according to manufacturer's protocol. Total RNA was quantified using Nanodrop 2000. Isolated RNA (1.4 ug) from skin tissue was used as a template in a 20 ul PCR volume using Invitrogen SuperScript VILO cDNA Synthesis kit to create cDNA template. The cDNA was diluted 1:25 for subsequent qPCR with the specific TaqMan probe for each gene to be quantified. Life Technologies A Vii7 PCR machine was used for the qPCR 40 amplification cycles. RNA levels of Cyp1a1 relative expression were calculated using the Delta CT formula and normalized to untreated skin sections.

Formulations 17, 21 and 22 and Formulation 12 showed biological activity for 3,5-Dihydroxy-4-isopropyl-trans-stilbene in human skin as measured indirectly by Cyp1A1 mRNA. There were no differences in the biological activity between these formulations. The lack of dose response from Formulations 17, 21 and 22 suggests this upregulation has reached a max or plateau that is likely achieved with formulations containing 0.5% 3,5-Dihydroxy-4-isopropyl-trans-stilbene or below. These data confirm the concentrations quantified in the in vitro skin penetration studies are bioavailable and capable of engaging targets within human skin. What has been found is that this assay does not provide statistically significant differences between formulations and/or concentrations of active in the formulations.

### Example 6—Systemic Exposure Anti Skin Concentrations of 3,5-Dihytlroxy-4-isopropyl-trans-stilbene Following 7-Day Topical Administration to Gottingen Minipigs

To assess tissue concentrations and systemic exposure, a toxicokinetics study in Gottingen mini pigs was conducted with repeat topical administration of 3,5-Dihydroxy-4-isopropyl-trans-stilbene over 7 days. To compare systemic exposure to local skin tissue levels, tissue biopsies were collected, sectioned, and analyzed for active ingredient and compared to plasma concentrations on Day 1 and Day 7. A similar methodology for skin biopsies in minipigs is described in the literature such as by Mitra A, et al., Use of Minipig Skin Biopsy Model as an Innovative Tool to Design Topical Formulation to Achieve Desired Pharmacokinetics in Humans. J Pharm Sci 2015, Feb. 17.

Formulation 22 (2% of active ingredient) and Formulation 12 (2.0%, e.g., formulation 14) were applied to the non-abraded skin of mini pigs (3 males/group) at a dose of 2% daily (for a period of approximately 23 hours) for 7 days. Dermal application sites for these groups were 10% of the total body surface area. The dose weight was 2 g/kg/day, as determined by formulation feasibility testing conducted prior to the start of dosing (the appropriate vehicle was applied to 10% of the total body surface area of the first mini pig in each of the aforementioned dose groups for approximately 23 hours). Hair was removed from the back and flanks of each animal on Days 1, 7 and 14, and as needed during the study. Application sites were semi-occluded with gauze patches held in place with tape and tubular netting. At the end of the exposure period, the netting and patches were removed, and all sites were wiped with warm reverse osmosis-treated water and cotton gauze.

The following endpoints/parameters were evaluated: clinical observations, dermal irritation (scored using the Draize method, body weights, and macroscopic and microscopic observations (treated and untreated skin). Toxicokinetic evaluation was performed on samples collected on Days 1 and 7 for plasma and skin concentrations.

On Day 7, the mini pigs were euthanized and the surface of the skin was cleaned through a combination of several wash steps including mild cleansers, solvents, shaving, and tape stripping to ensure residual formulation was removed from the surface of the skin. The skin was then excised and skin was placed epidermis side down on a clean surface. Skin biopsies (8 mm) were harvested from the dosing areas, placed in cryotubes, and immediately frozen until analysis. While still frozen, the hypodermis was removing using a razor blade and the upper section of the sample (epidermis and upper dermis, section from 0 to 500 μm) was then cut away from the dermis. Therefore, epidermis refers to the upper section of the skin sample consisting of stratum corneum, epidermis, and upper dermis (approximately 500 μm) and dermis represents deeper dermis (approximately 1,500 μm meaning a cut of middle dermis to subcutaneous fat). The epidermis and dermis sections were then weighed and homogenized in 1 ml of a 75:25 water:acetonitrile solution containing 0.1% formic acid. The homogenate was further processed by protein precipitation using a solution of 100% acetonitrile with 0.1% formic acid and an internal standard (5 ng/ml). The supernatant from the protein precipitation was passed through an Ostro 96 well plate to remove phospholipids.

Formulation 22 resulted in higher skin deposition of 3,5-Dihydroxy-4-isopropyl-trans-stilbene, in the upper 500 μm and lower 1,500 μm sections, compared to Formulation 12 (with 2% active ingredient, also referred to herein as Formulation 14). The increase in the skin loading was observed on Day 1 and continued after 7 days of repeat dosing. This data suggests Formulation 22 has the capability of changing the skin microenvironment after a single application but can also maintain this effect over longer dosing periods.

Surprisingly, the systemic exposure as measured by AUC on Day 1 and Day 7 was lower for Formulation 22 compared to Formulation 12 (with 2% active ingredient, e.g., Formulation 14). The plasma concentrations seemed to decrease further for Formulation 22 by Day 7, where the levels were approximately 2.5 fold lower for Formulation 22 compared to Formulation 12 (with 2% active ingredient, e.g., Formulation 14). The data correlates with what was observed in the in vitro human skin penetration studies where Formulation 22 showed higher skin deposition with lower concentrations of 3,5-Dihydroxy-4-isopropyl-trans-stilbene delivered to the receiving fluid. The combination of this data suggests that Formulation 22 is capable of delivering higher tissue amounts to the target site, while minimizing systemic exposure.

Based on in vitro skin penetration flux and human data, predicted human AUCs are expected be below 50 ng*h/mL, or below 42.5 ng*h/mL and the predicted Cₘₐₓ is expected to be below 15 ng/ml or 12.5 ng/mL.

TABLE 4

| | AUC | | | Cmax | | |
|---|---|---|---|---|---|---|
| | NOAEL | Predicted | Margin of | NOAEL | Predicted | Margin of |
| Nonclinical Study | (ng · h/mL) | (ng · h/mL) | Safety | (ng/mL) | (ng/mL) | safety |
| Dermal 4% cream, BID, Minipig 13 weeks | 99 | 42.5 | 2 | 7.13 | 12.5 | <1 |
| Subcutaneous 3 mg/kg/day, Rat, 13 weeks | 99 | 42.5 | 2 | 31.6 | 12.5 | 2.5 |

Based on in vivo mini pig data and human data, the predicted human AUCs are expected to be below 30.0 ng*h/mL, or below 23.5 ng*h/mL administered at a 2% w/w strength of active to the human skin. Based on in vivo minipig data and human data, the predicted C1111 1× is expected to be below 15 ng/ml or below 11.3 ng/mL administered at a 2% w/w strength of active to the human skin.

Based now upon limited human data, human AUCs (0-8h) are expected to be below 16.0 ng*h/mL, or below 14 ng*h/mL, or below 11 ng*h/mL administered at a 1% w/w strength of active to the human skin. In another embodiment, the Cₘₐₓ is expected to be below 5 ng/ml, or below 4 ng/ml, or below 3 ng/ml, administered at a 1% w/w strength of active to the human skin.

TABLE 5

| | AUC | | | Cmax | | |
|---|---|---|---|---|---|---|
| | NOAEL | Predicted | Margin of | NOAEL | Predicted | Margin of |
| Nonclinical Study | (ng · h/mL) | (ng · h/mL) | Safety | (ng/mL) | (ng/mL) | safety |
| Dermal 4% cream, BID, Minipig 13 weeks | 99 | 23.5 | 4 | 7.13* | 11.3 | ~1 |
| Subcutaneous 3 mg/kg/day, Rat, 13 weeks | 99 | 23.5 | 4 | 31.6 | 11.3 | 3 |

### Example 7—Systemic Exposure and Skin Concentrations of 3,5-Dihydroxy-4-isopropyl-trans-stilbene Following 28-Day Topical Administration to Gottingen Minipigs

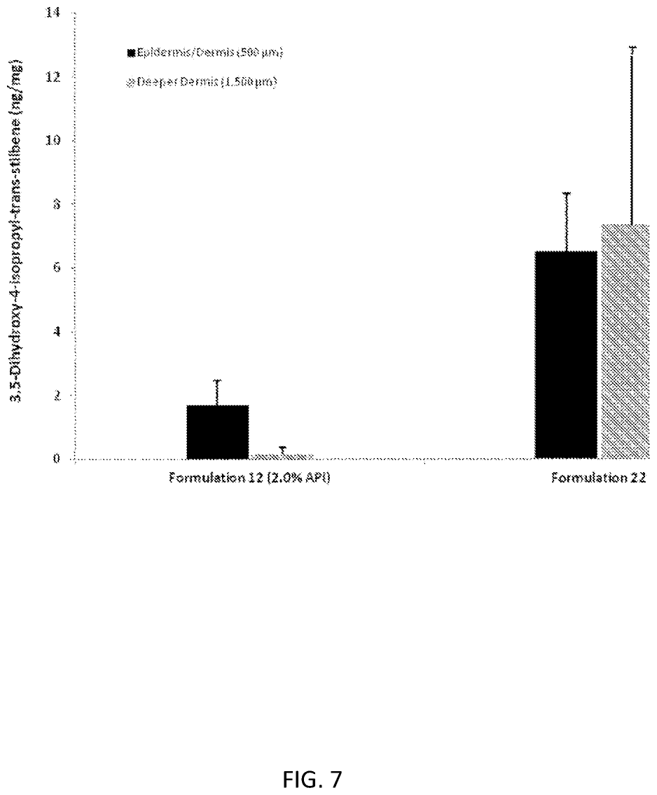

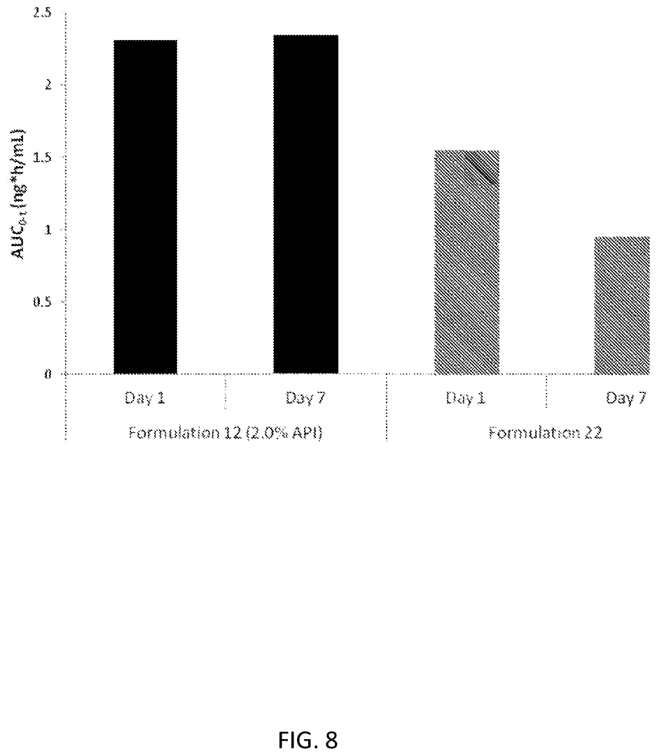

The 7-day topical administration of 3,5-Dihydroxy-4-isopropyl-trans-stilbene in Formulation 22 showed significant concentrations of 3,5-Dihydroxy-4-isopropyl-trans-stilbene in the skin of Gottingen mini pigs. See FIG. 7. The systemic levels (FIG. 8) from this study decreased from Day 1 to Day 7, suggesting Formulation 22 is capable of delivering higher tissue amounts to the target site, while minimizing systemic exposure. To see if this trend would continue over a longer dosing period, a repeat dosing of topical administration of 3,5-Dihydroxy-4-isopropyl-trans stilbene over 28 days was conducted to assess the tissue concentrations, dermal toxicity, dermal irritancy and toxicokinetics from Formulation 22.

The formulations were administered as a twice daily (10±1 hours apart) topical application to the dorsal skin (˜10% of total body surface area) of Gottingen minipigs (3/sex/group) for 28 days and the dermal toxicity, dermal irritancy and of 3,5-Dihydroxy-4-isopropyl-trans-stilbene was determined. The initial daily doses were 0 (vehicle), 10, 20 or 60 mg/kg/day 3,5-Dihydroxy-4-isopropyl-trans-stilbene (concentrations at 0, 0.5, 1.0 and 3.0% w/w, respectively) at a dose formulation weight of 1 g/kg/dose (2 g/kg/day). Following dose application on each dosing day, the dose sites were semi-occluded for approximately 20±1 hours from the first dose and then gently washed before the next dose was applied.

TABLE 6
Mean Plasma and Skin Toxicokinetic Parameters Following Twice Daily Dermal Administration of 3,5-
Dihydroxy-4-isopropyl-trans-stilbene in Formulation 22 to Male and Female Gottingen Minipigs

| | Male | | | | Female | | | |
|---|---|---|---|---|---|---|---|---|
| | Daily Dose (mg/kg/day)^{a} | | | | | | | |
| | 0 | 10 | 20 | 60 | 0 | 10 | 20 | 60 |
| Numbers of Animals: | 3 | 3 | 3 | 3 | 3 | 3 | 3 | 3 |
| AUC₍₀₋ₜ₎ (ng · h/mL) Day 1 | NA | NA | NA | NR | NA | NR | NA | NR |
| Day 28 | NA | NA | NA | NR | NA | NR | NA | 4.84 |
| Cₘₐₓ (ng/mL) Day 1 | NA | NA | NA | 0.376 | NA | NR | NA | 0.408 |
| Day 28 | NA | NA | NA | 0.845 | NA | 0.395 | NA | 0.830 |
| Skin Concentration (Day 29; ng/g) | | | | | | | | |
| Epidermis/Upper Dermis | — | 4830 | 3950 | 10000 | — | 2540 | 2270 | 6530 |
| Lower Dermis | — | 166 | 615 | 3660 | — | NQ | 305 | 3.04 |

On Days 1 and 28, 3,5-Dihydroxy-4-isopropyl-trans-stilbene was not quantifiable in plasma at 5 and 10 mg/kg/dose (10 and 20 mg/kg/day) except for 2 time points on Day 28 (3 and 24 hours post first dose) which had 1 concentration value at each time point for females at 5 mg/kg/dose (10 mg/kg/day). At 30 mg/kg/dose (60 mg/kg/day), 3,5-Dihydroxy-4-isopropyl-trans-stilbene was quantifiable in plasma up to 3 hours after dosing (either first or second dose) on Days 1 and 28.

The Tₘₐₓ values ranged from 1 to 3 hours post-dose (either first or second dose) on Days 1 and 28 at the 60 mg/kg/day dose level. The Cₘₐₓ values ranged from 0.306 to 1.81 ng/ml for males and females. Generally, AUC₀₋ₜ could not be reported due to the majority of the concentrations values being lower than the limit of quantification.

3,5-Dihydroxy-4-isopropyl-trans-stilbene concentrations were generally higher in the epidermis/upper dermis of both males and females than those in the lower dermis. The gender-averaged individual animal skin concentrations at necropsy were highest in the 60 mg/kg/day group at 8265 ng/g in the epidermis/upper dermis and 1830 ng/g in the lower dermis. Comparison of the concentrations in the skin to those in the plasma could not be assessed since the Day 28 24-hour plasma concentrations were below the limit of quantification.

All animals survived to their scheduled necropsy. There was no test article-related dermal irritation based on dermal evaluation of the dose site. There was no systemic toxicity based on clinical observations, cardiovascular (ECG) and ophthalmoscopic evaluations, body weight, food consumption, clinical pathology and post-mortem evaluations (organ weights, macroscopic and microscopic pathology). Thus, one embodiment of the invention is a nonirritating or reduced irritating pharmaceutical composition comprising 3,5-Dihydroxy-4-isopropyl-trans-stilbene or a pharmaceutically acceptable salt thereof, as compared to a formulation having a composition of 1 or 12 (with comparable % w/w active).

In conclusion, following twice daily topical administration of 3,5-Dihydroxy-4-isopropyl-trans-stilbene at doses of 0 (vehicle), 0.5, 10 and 3.0% w/w (animal study) in the present inventive formulations described in Example 7 (10, 20 mg/kg/day) and Example 8 (60 mg/kg/day, respectively) onto ˜10% of total body surface area of mini pigs (n=3/sex/group) for 28 days, resulted in no dermal irritation or systemic toxicity. Therefore the “No Observed Adverse Effect Level” (NOAEL) for dermal irritation or systemic toxicity was 3% (w/w) or 60 mg/kg/day, the highest dose tested. 3,5-Dihydroxy-4-isopropyl-trans-stilbene achieved a peak average concentration of 8265 ng/g in the skin (epidermis/upper dermis) in the 60 mg/kg/day group while systemic exposures were relatively much lower or often below the limit of quantitation in all treatment groups.

### Example 8: Development of Additional Formulations Containing High Co-Solvents

The Sponsor developed a cream formulation (CR01) containing tapinarof (DMVT-505) as a topical product for the potential treatment of psoriasis and atopic dermatitis. Additional formulations were manufactured with differing aqueous (WS3) phases, and different emulsifiers, oils and co-solvents from CR01 for in vitro permeation and penetration test (IVPT) evaluation.

Pre-formulation experiments and the development of formulations (including short-term stability) for use in performance testing to achieve the above aim were performed.

Pre-formulation experiments were performed to understand the solubility and stability of the drug in excipients. In relation to drug/excipient compatibility, DMVT-505 was stable for 2 weeks (peak purity>98%) in most excipients at 40° C., with the exception of MCTs and DMI, which may be due to oxidation. Based on the pre-formulation data, aqueous-based solvent systems were designed for WS3. Further formulations were prepared combining suitable phases developed during all the workstreams to obtain formulations to limit the suspected drug partitioning between the aqueous and oil phases (WS2/3/4).

Formulation candidates, pooled from all workstreams, were then selected for short-term (2 week) stability testing. Formulations were assessed for drug content/purity, visual appearance, microscopic appearance with droplet sizing, apparent pH, Brookfield viscosity and LUMiSizer (at 40° C. at t=0 only). Drug in the majority of formulations appeared chemically stable over the 2-week period (purity>99%), except a small number of the formulations containing alternative antioxidants (i.e., without EDTA and another formulation with ascorbic acid) for which a slight drop in drug purity was observed. However, as a color change was observed in active formulations from WS3 and WS2/3/4 formulations containing PEG 400 and Transcutol P (e.g., CR47) and not corresponding placebo formulation, there may be slight drug degradation which cannot be detected on the current analytical method.

Formulations were evaluated using IVPT with ex vivo human skin mounted on flow through diffusion cells (MedFlux-HT®). A total of four donors were used to test all formulations, and CR01 was applied on each donor as a bridge formulation. The intent of this testing was to compare the permeation and penetration of the test formulations to CR01, which all contained 1% w/w DMVT-505. Receptor solution (citrate phosphate buffer (pH 4.0)+0.01% Brij O20 w/v+0.1% sodium azide+20 mM ascorbic acid) and extraction fluid (90:10 v/v acetonitrile:water) developed previously. Receptor solution was collected every 2 hours for 36 hours and tissue was harvested at the 36-hour timepoint.

Summary tables listing the results of the formulations tested regarding physical characteristics and skin permeation or penetration are provided below.

### Development of Alternative Aqueous-Based Solvent Systems (WS3)

Formulations are provided in Table 7.

Formulations tested in Table 8 were designed combining previously tested solvent systems. A range of solvent systems were selected based on compositional differences (e.g., with or without volatile solvent) and DMVT-505 solubility profiles. When manufacturing active formulations, the active formulations showed a delay in thickening compared to placebos and in some cases a decrease physical stability and this may be due to the drug having an impact on the physical properties of the emulsion. Therefore, it was decided to focus formulation development efforts in obtaining stable active formulations rather than placebos.

The formulations were characterized and the results are shown in Table 8.

A number of formulations were shown to be suitable for further development and testing (e.g., in a short-term stability experiment), including CR47, CR50, and CR51. While for these formulations accelerated physical stability testing indicated that the formulations phase separated following the first 2 minute cycle, analysis of the LUMiSizer data (transmission profile and stability index) suggests that these formulations may be at least as physically stable as CR01.

Droplet size analysis revealed that CR01 has a lower droplet size when compared to the developed WS3 active formulations. However, it should be noted that there are differences in the scale at which these formulations were prepared and different manufacturing elements (e.g. homogeniser head, screens etc.) that may have been used could explain this.

The droplet distribution and homogeneity broadly correlated with the LUMiSizer data.

TABLE 7
Formulations with alternative aqueous phases

| | CR47 | CR50 | CR51 |
|---|---|---|---|
| Excipients and API | ACT | ACT | ACT |
| Solvent system | SS2 | SS11 | SS13 |
| DMVT-505 | 1.00 | 1.00 | 1.00 |
| Propylene glycol | 7.77 | 15.53 | 15.53 |
| Transcutol P | 15.53 | 15.53 | 7.77 |
| PEG 400 | 15.53 | 15.53 | 15.53 |
| Ethanol | — | — | — |
| Arlasolve DMI (dimethyl | — | — | 7.77 |
| isorbide) | | | |
| Isopropyl alcohol (IPA) | — | — | — |
| Emulsifying wax. Nonionic | 7.20 | 7.20 | 7.20 |
| (Polawax NF-PA-MH | | | |
| Medium chain triglycerides | 10.00 | 10.00 | 10.00 |
| (Miglyol 810) | | | |
| Tween 80 | 1.50 | 1.50 | 1.50 |
| Steareth 2, pastellated | 1.80 | 1.80 | 1.80 |
| Steareth 20, pastellated | 1.10 | 1.10 | 1.10 |
| Benzoic acid | 0.25 | 0.25 | 0.25 |
| Butylated hydroxytoluene | 0.10 | 0.10 | 0.10 |
| (BHT) | | | |
| Water | 37.83 | 30.07 | 30.07 |
| Sodium citrate dihydrate | 0.19 | 0.19 | 0.19 |
| Citric acid monohydrate | 0.09 | 0.09 | 0.09 |
| EDTA- di-sodium | 0.10 | 0.10 | 0.10 |
| Total | 100.00 | 100.00 | 100.00 |
| Solvent system solubility | 0.813 | 4.246 | 2.710 |
| (% w/w) | | | |
| Formulation suitable | Y | Y | Y |

TABLE 8
Physical Characteristics of formulations with alternative aqueous phases

| | | | | | | Mean | | |
|---|---|---|---|---|---|---|---|---|
| | | | | | | Cumulative | | |
| | | Droplet | | | Mean | Amount | | |
| | | sizing | | | average | DMVT-505 | Penetration | Penetration |
| | | (diameter, | | Instability | viscosity | delivered to | in Epidermis | in Dermis |
| Formulation | Description | n = 6) | | Index | (mPa · s) | receptor(ng/cm²) | (ng)-Mean | (ng)-Mean |
| CR01 PBO | | | | 0.018 | | | | |
| CR01 ACT | Uniform | 3.54 | μm | 0.061 | | | | |
| MP | distribution | 3.81 | μm | | | | | |
| | of regular | 3.74 | μm | | | | | |
| | sized droplets | 3.25 | μm | | | | | |
| | Uniform | 3.11 | μm | | | | | |
| | distribution | 3.39 | μm | | | | | |
| | of regular | | | | | | | |
| | sized droplets | | | | | | | |
| CR01 ACT | Uniform | 0.60 | μm | 0.085 | 148,667 | 25.15, 39.65, | 997, 1338, | 2976, 5112, |
| Sponsor | distribution | 0.78 | μm | | | 21.23, 23.94 | 534, 4814 | 3836, 4308 |
| | of regular | 0.44 | μm | | | depending | depending | depending |
| | sized droplets | 0.71 | μm | | | on which | on which | on which |
| | | 0.44 | μm | | | trial and | trial and | trial and |
| | | 0.70 | μm | | | skin donor | skin donor | skin donor |
| CR47 ACT | Uniform | 5.93 | μm | 0.047 | 134,500 | 19.33* | 1167 | 3460 |
| | distribution | 4.83 | μm | | | | | |
| | of disperse | 6.25 | μm | | | | | |
| | phase, mostly | 4.50 | μm | | | | | |
| | regular | 4.49 | μm | | | | | |
| | droplets | 8.56 | μm | | | | | |
| CR50 ACT | Uniform | 10.50 | μm | 0.046 | 204,000 | 38.48 | 1392 | 2596 |
| | distribution | 9.49 | μm | | | | | |
| | of disperse | 14.14 | μm | | | | | |
| | phase, mostly | 10.26 | μm | | | | | |
| | regular | 14.50 | μm | | | | | |
| | droplets | 9.99 | μm | | | | | |
| CR51 ACT | Uniform | 4.69 | μm | 0.048 | 191,500 | 16.58* | 1662 | 2402* |
| | distribution | 4.21 | μm | | | | | |
| | of disperse | 4.69 | μm | | | | | |
| | phase, mostly | 4.50 | μm | | | | | |
| | regular | 5.25 | μm | | | | | |
| | droplets | 5.22 | μm | | | | | |

*statistical significance compared with CR01 at p < 0.05

### Development of Formulations Combining Workstreams (WS2/3/4)

Formulations are provided in Tables 9A-9E

CR44 and CR45 were based on CR34 (SSO13) for the oil phase as it showed good physical stability during WS 3. SS5, SS13 and SS15 were employed in the development of the aqueous phases to increase the solubility of DMVT-505 in that phase and displace the partitioning to the oil phase. Similarly, CR55 was based in the same solvent systems utilizing the emulsifiers used in CR17.

CR58, CR59, and CR61 were based on solvent systems from WS3 in which the API had greater solubility than 1%, with mineral oil as the oil phase to limit the partition of the API into the oil phase (as mineral oil is a non-solvent for DMVT-505). CR58 and CR59 contained the emulsifier system in CR17.

CR64 was developed based on the original aqueous system, combined with SSO12 aiming to increase DMVT-505 thermodynamic activity by reducing the solubility in the overall system, while maintaining the target dose. The emulsifiers from CR17 were utilized as they were amongst the most successful in obtaining a physically stable formulation.

CR70, CR71, CR79 and CR80 are formulations containing diisopropyl adipate in the oil phase, a good solubilizer for DMVT-505, PEG 100 stearate, PEG 8 stearate and Tefose 63 were among the emulsifiers employed. Changes were made in the aqueous phase (e.g., removal of Transcutol P) and in the preservative system.

Physical characteristics for each formulation are provided in Table 10.

TABLE 9A
Composition of formulations from combined workstreams

| | Original | CR45 | CR55 |
|---|---|---|---|
| Excipients* and API | (CR01) | ACT | ACT |
| DMVT-505 | 1.00 | 1.00 | 1.00 |
| Propylene glycol | 10.00 | 15.53 | 15.53 |
| Transcutol P | 2.00 | 7.77 | 7.77 |
| PEG 400 | — | 15.53 | 15.53 |
| Ethanol | — | — | — |
| DMI | — | 7.77 | 7.77 |
| IPA | — | — | — |
| Emulsifying wax. Nonionic | 7.20 | 7.20 | 7.20 |
| (Polawax NF) | | | |
| Medium chain triglycerides | 10.00 | 2.00 | 2.00 |
| (Crodamol GTCC) | | | |
| Mineral oil | — | 8.00 | 8.00 |
| Polysorbate 80 (Tween 80) | 1.50 | 1.50 | — |
| Polysorbate 60 (Tween 60) | — | — | 1.50 |
| Steareth 2 (Brij S2) | 1.80 | 2.10 | 2.10 |
| Steareth 20 (Brij S20) | 1.10 | 0.80 | — |
| Steareth 21 (Brij S721) | — | — | 0.80 |
| Benzoic acid | 0.25 | 0.25 | 0.25 |
| Butylated hydroxytoluene | 0.10 | 0.10 | 0.10 |
| (BHT) | | | |
| Water | 64.67 | 30.07 | 30.07 |
| Sodium citrate dihydrate | 0.19 | 0.19 | 0.19 |
| Citric acid monohydrate | 0.09 | 0.09 | 0.09 |
| EDTA- di-sodium | 0.10 | 0.10 | 0.10 |
| Total | 100.00 | 100.00 | 100.00 |
| WS | N/A | 2-3 | 2-3-4 |
| Formulation suitable | N/A | (Y) | (Y) |

TABLE 9B
Composition of formulations from combined workstreams

| | Original | CR58 | CR58 | CR59 |
|---|---|---|---|---|
| Excipients and API | (CR01) | ACT | PBO | ACT |
| DMVT-505 | 1.00 | 1.00 | — | 1.00 |
| Propylene glycol | 10.00 | 15.53 | 15.53 | 15.53 |
| Transcutol P | 2.00 | 15.53 | 15.53 | 7.77 |
| PEG 400 | — | 15.53 | 15.53 | 15.53 |
| Ethanol | — | — | — | — |
| Arlasolve DMI (dimethyl | — | — | — | 7.77 |
| isorbide) | | | | |
| IPA | — | — | — | — |
| Emulsifying wax. | 7.20 | 7.20 | 7.20 | 7.20 |
| Nonionic (Polawax NF) | | | | |
| Medium chain | 10.00 | — | — | — |
| triglycerides | | | | |
| (Miglyol 810) | | | | |
| Mineral oil | — | 10.00 | 10.00 | 10.00 |
| Polysorbate 80 (Tween | 1.50 | — | — | — |
| 80) | | | | |
| Polysorbate 60 (Tween | — | 1.50 | 1.50 | 1.50 |
| 60) | | | | |
| Steareth 2 (Brij S2) | 1.80 | 2.20 | 2.20 | 2.20 |
| Steareth 20 (Brij S20) | 1.10 | — | — | — |
| Steareth 21 (Brij S721) | — | 0.70 | 0.70 | 0.70 |
| Benzoic acid | 0.25 | 0.25 | 0.25 | 0.25 |
| Butylated | 0.10 | 0.10 | 0.10 | 0.10 |
| hydroxytoluene (BHT) | | | | |
| Water | 64.67 | 30.08 | 31.08 | 30.07 |
| Sodium citrate dihydrate | 0.19 | 0.19 | 0.19 | 0.19 |
| Citric acid monohydrate | 0.09 | 0.09 | 0.09 | 0.09 |
| EDTA- di-sodium | 0.10 | 0.10 | 0.10 | 0.10 |
| Total | 100.00 | 100.00 | 100.00 | 100.00 |
| WS | N/A | 2-3-4 | 2-3-4 | 2-3-4 |
| Formulation suitable | N/A | (Y) | (Y) | Y |

TABLE 9C
Composition of formulations from combined workstreams

| | CR01 | CR61 | CR64 |
|---|---|---|---|
| Excipients and API | ACT | ACT | ACT |
| DMVT-505 | 1.00 | 1.00 | 1.00 |
| Propylene glycol | 10.00 | 15.53 | 10.00 |
| Transcutol P | 2.00 | 15.53 | 2.00 |
| PEG 400 | — | 15.53 | — |
| Ethanol | — | — | — |
| Arlasolve DMI (dimethyl isosorbide) | — | — | — |
| Emulsifying wax. Nonionic | 7.20 | — | — |
| (Polawax NF) | | | |
| Cetostearyl alcohol | — | 6.60 | — |
| Cetyl alcohol | — | — | 8.00 |
| Medium chain triglycerides (Miglyol | 10.00 | — | 5.00 |
| 810) | | | |
| Mineral oil | — | 10.00 | 5.00 |
| Polysorbate 60 (Tween 60) | — | — | — |
| Polysorbate 80 (Tween80) | 1.50 | — | — |
| Steareth 2 (Brij S2) | 1.80 | — | — |
| Steareth 20 (Brij S20) | 1.10 | — | — |
| Steareth 21 (Brij S721) | — | — | — |
| Arlacel 165 | — | 5.00 | — |
| Tefose 63 | — | — | — |
| Labrafil M 1944 | — | — | — |
| Gelot 64 | — | — | 4.00 |
| Benzoic acid | 0.25 | 0.25 | 0.250 |
| Butylated hydroxytoluene (BHT) | 0.10 | 0.10 | 0.10 |
| Water | 64.67 | 30.08 | 64.27 |
| Sodium citrate dihydrate | 0.19 | 0.19 | 0.19 |
| Citric acid monohydrate | 0.09 | 0.09 | 0.09 |
| EDTA- di-sodium | 0.10 | 0.10 | 0.10 |
| Total | 100.00 | 100.00 | 100.00 |
| WS | N/A | 2-3-4 | 2-4 |
| Formulation suitable | N/A | (Y) | (Y) |

TABLE 9D
Composition of formulations from combined workstreams

| | CR01 | CR70 | CR71 |
|---|---|---|---|
| Excipients and API | (Original) | ACT | ACT |
| DMVT-505 | 1.00 | 1.00 | 1.00 |
| Propylene glycol | 10.00 | 10.00 | — |
| Transcutol P | 2.00 | — | — |
| PEG 400 | — | — | — |
| Arlasolve DMI (dimethyl | — | — | — |
| isorbide) | | | |
| Emulsifying wax. Nonionic | 7.20 | — | — |
| (Polawax NF-PA-MH) | | | |
| Medium chain tryglycerides | 10.00 | — | — |
| Stearyl alcohol | — | 0.28 | — |
| Mineral oil | 3.00 | 3.00 | 3.00 |
| Arlacel 165 | — | — | — |
| DIPA | — | 10.00 | 10.00 |
| GMS-II (Kolliwax GMS-II) | — | — | — |
| Polysorbate 60 (Tween 60) | — | 1.33 | — |
| Polysorbate 80 (Tween 80) | 1.50 | — | — |
| Steareth 2 (Brij S2) | 1.80 | — | — |
| Steareth 20 (Brij S721) | 1.10 | — | — |
| PEG 100 Stearate | — | — | — |
| PEG 8 stearate | — | — | — |
| Span 60 | — | 7.89 | — |
| Tefose 63 | — | — | 20.00 |
| Labrafil M 1944 | — | — | 3.00 |
| Benzyl alcohol | — | 1.00 | 1.00 |
| Benzoic acid | 0.25 | — | — |
| Butylated hydroxytoluene | 0.10 | 0.10 | 0.10 |
| (BHT) | | | |
| HEC (HHX) | — | — | — |
| Water | 64.67 | 65.30 | 61.80 |
| EDTA- di-sodium | 0.10 | 0.10 | 0.10 |
| Sodium citrate dihydrate | 0.19 | — | — |
| Citric acid monohydrate | 0.09 | — | — |
| Total | 100.00 | 100.00 | 100.00 |
| WS | N/A | 2-3-4 | 2-3-4 |
| Formulation suitable | N/A | (Y) | Y |
| Apparent pH | N/A | 6.75 | 3.23 |

TABLE 9E
Composition of formulations from combined workstreams

| | CR01 | CR76 | CR77 | CR78 | CR79 | CR80 |
|---|---|---|---|---|---|---|
| Excipients and API | (Original) | ACT | ACT | ACT | ACT | ACT |
| DMVT-505 | 1.00 | 1.00 | 1.00 | 1.00 | 1.00 | 1.00 |
| Propylene glycol | 10.00 | 14.85 | 15.01 | 14.85 | — | 10.00 |
| Transcutol P | 2.00 | 7.43 | 7.51 | 7.43 | — | — |
| PEG 400 | — | 14.85 | 15.01 | 14.85 | — | — |
| Arlasolve DMI | — | 7.43 | 7.51 | 7.43 | — | — |
| (dimethyl isorbide) | | | | | | |
| Emulsifying wax. Nonionic | 7.20 | — | — | — | — | — |
| (Polawax NF-PA-MH) | | | | | | |
| Medium chain tryglycerides | 10.00 | — | — | — | — | — |
| Stearyl alcohol | — | 7.00 | 7.00 | 7.00 | 4.55 | 4.55 |
| Mineral oil | — | 10.00 | 10.00 | 10.00 | 3.00 | 3.00 |
| Polysorbate 80 (Tween 80) | 1.50 | — | — | — | — | — |
| Steareth 2 (Brij S2) | 1.80 | — | — | — | — | — |
| Steareth 21 (Brij S721) | 1.10 | — | — | — | — | — |
| Arlacel 165 | — | 6.00 | 6.00 | 6.00 | — | — |
| DIPA | — | — | — | — | 10.00 | 10.00 |
| GMS-II (Kolliwax GMS-II) | — | — | — | — | 6.70 | 6.70− |
| PEG 100 Stearate | — | — | — | — | 8.75 | 8.75 |
| Benzyl alcohol | — | — | — | — | 1.00 | 1.00 |
| Benzoic acid | 0.25 | 0.25 | 0.25 | 0.25 | — | — |
| Butylated hydroxytoluene | 0.10 | 0.10 | 0.10 | 0.10 | 0.10 | 0.10 |
| (BHT) | | | | | | |
| HEC (HHX) | — | 0.20 | — | — | — | — |
| Carbopol 980 | — | — | 0.10 | — | 0.20 | — |
| Water | 64.67 | 30.51 | 20.41 | 30.71 | 63.87 | 54.90 |
| EDTA- di-sodium | 0.10 | 0.10 | 0.10 | 0.10 | — | — |
| Sodium citrate dihydrate | 0.19 | 0.19 | — | 0.19 | — | — |
| Citric acid monohydrate | 0.09 | 0.09 | — | 0.09 | — | — |
| 0.1M NaOH solution | — | — | pH 6-7 | — | 0.83 | — |
| 2nd Water addition | — | — | Q.S to 100% | — | — | — |
| Total | 100.00 | 100.00 | 100.00 | 100.00 | 100.00 | 100.00 |
| WS | N/A | 2-3-4 | 2-3-4 | 2-3-4 | 2-3-4 | 2-3-4 |
| Formulation suitable | N/A | Y | Y | (Y) | Y | Y |

TABLE 10
Physical Characteristics of formulations from combined workstreams

| | | | | | | Mean | | |
|---|---|---|---|---|---|---|---|---|
| | | | | | | Cumulative | | |
| | | Droplet | | | Mean | Amount | | |
| | | sizing | | | average | DMVT-505 | Penetration | Penetration |
| | | (diameter, | | Instability | viscosity | delivered to | in Epidermis | in Dermis |
| Formulation | Description | n = 6) | | Index | (mPa · s) | receptor(ng/cm²) | (ng)-Mean | (ng)-Mean |
| CR01 PBO | | | | 0.018 | | | | |
| CR01 ACT | Uniform | 3.54 | μm | 0.061 | | | | |
| MP | distribution | 3.81 | μm | | | | | |
| | of regular | 3.74 | μm | | | | | |
| | sized droplets | 3.25 | μm | | | | | |
| | | 3.11 | μm | | | | | |
| | | 3.39 | μm | | | | | |
| CR01 ACT | Uniform | 0.60 | μm | 0.085 | 148,667 | 25.15, 39.65, | 997, 1338, | 2976, 5112, |
| Sponsor | distribution | 0.78 | μm | | | 21.23, 23.94 | 534, 4814 | 3836, 4308 |
| | of regular | 0.44 | μm | | | depending | depending | depending |
| | sized droplets | 0.71 | μm | | | on which | on which | on which |
| | | 0.44 | μm | | | trial and | trial and | trial and |
| | | 0.70 | μm | | | skin donor | skin donor | skin donor |
| CR45 ACT | Uniform | 7.00 | μm | 0.082 | 177,500 | 9.66 | 1752* | 5960 |
| | distribution | 7.76 | μm | | | | | |
| | of disperse | 7.64 | μm | | | | | |
| | phase, mostly | 6.38 | μm | | | | | |
| | regular | 8.42 | μm | | | | | |
| | droplets | 8.82 | μm | | | | | |
| CR55 ACT | Uniform | 18.42 | μm | 0.072 | 349,545 | 11.32 | 1731* | 5150 |
| | distribution | 12.21 | μm | | | | | |
| | of disperse | 13.56 | μm | | | | | |
| | phase, | 10.16 | μm | | | | | |
| | irregular | 11.73 | μm | | | | | |
| | droplets | 17.08 | μm | | | | | |
| CR58 ACT | Non-uniform | 10.26 | μm | 0.037 | 906,083 | 20.18 | 4044* | 3504 |
| | distribution | 10.78 | μm | | | | | |
| | of disperse | 8.98 | μm | | | | | |
| | phase, | 5.25 | μm | | | | | |
| | irregular | 12.60 | μm | | | | | |
| | droplets | 12.69 | μm | | | | | |
| CR58 PBO | Non-uniform | 12.19 | μm | 0.016 | 1,051,750 | | | |
| | distribution | 9.59 | μm | | | | | |
| | of disperse | 9.39 | μm | | | | | |
| | phase, | 10.81 | μm | | | | | |
| | irregular | 9.74 | μm | | | | | |
| | droplets | 11.00 | μm | | | | | |
| CR59 ACT | Non-uniform | 13.61 | μm | 0.042 | 734,417 | 11.09 | 1299 | 2152 |
| | distribution | 14.75 | μm | | | | | |
| | of disperse | 31.23 | μm | | | | | |
| | phase, | 9.49 | μm | | | | | |
| | irregular | 13.48 | μm | | | | | |
| | droplets | 26.90 | μm | | | | | |
| CR61 ACT | Uniform | 3.42 | μm | 0.013 | 849,000 | 13.06 | 1524 | 2392 |
| | distribution | 3.89 | μm | | | | | |
| | of disperse | 4.78 | μm | | | | | |
| | phase, mostly | 5.05 | μm | | | | | |
| | regular | 3.83 | μm | | | | | |
| | droplets | 4.86 | μm | | | | | |
| CR64 ACT | Non-uniform | 15.76 | μm | 0.006 | 308,500 | 279.00* | 2204* | 6428 |
| | distribution | 12.01 | μm | | | | | |
| | of disperse | 15.39 | μm | | | | | |
| | phase, | 12.39 | μm | | | | | |
| | irregular | 14.43 | μm | | | | | |
| | droplets | 16.30 | μm | | | | | |
| CR70 ACT | Non-uniform | 12.73 | μm | 0.049 | 10,718 | 145.55* | 1958* | 2658 |
| | distribution | 12.67 | μm | | | | | |
| | of disperse | 14.58 | μm | | | | | |
| | phase, | 13.07 | μm | | | | | |
| | irregular | 11.42 | μm | | | | | |
| | droplets | 12.12 | μm | | | | | |
| CR71 ACT | Uniform | 9.17 | μm | 0.006 | 1,057,083 | 34.25 | 2696 | 12108* |
| | distribution | 11.35 | μm | | | | | |
| | of disperse | 10.23 | μm | | | | | |
| | phase, | 8.91 | μm | | | | | |
| | irregular | 9.06 | μm | | | | | |
| | droplets | 8.76 | μm | | | | | |
| CR76 ACT | Uniform | 3.35 | μm | 0.022 | 938,917 | 7.61* | 2624 | 1635* |
| | distribution | 5.51 | μm | | | | | |
| | of disperse | 1.22 | μm | | | | | |
| | phase, mostly | 4.40 | μm | | | | | |
| | regular | 6.30 | μm | | | | | |
| | droplets | 5.20 | μm | | | | | |
| CR77 ACT | Uniform | 8.67 | μm | 0.019 | | | | |
| | distribution | 10.40 | μm | | | | | |
| | of disperse | 9.46 | μm | | | | | |
| | phase, | 7.70 | μm | | | | | |
| | irregular | 10.14 | μm | | | | | |
| | droplets | 11.13 | μm | | | | | |
| CR78 ACT | Non-uniform | 3.31 | μm | 0.042 | | | | |
| | distribution | 1.91 | μm | | | | | |
| | of disperse | 4.27 | μm | | | | | |
| | phase, mostly | 2.83 | μm | | | | | |
| | regular | 2.21 | μm | | | | | |
| | droplets | 2.45 | μm | | | | | |
| CR79 ACT | Uniform | 5.69 | μm | 0.010 | | | | |
| | distribution | 6.04 | μm | | | | | |
| | of disperse | 6.04 | μm | | | | | |
| | phase, | 5.35 | μm | | | | | |
| | irregular | 6.90 | μm | | | | | |
| | droplets | 5.52 | μm | | | | | |
| CR80 ACT | Uniform | 4.83 | μm | 0.010 | 276,583 | 13.37 | 3374 | 6550 |
| | distribution | 5.52 | μm | | | | | |
| | of disperse | 4.83 | μm | | | | | |
| | phase, mostly | 4.48 | μm | | | | | |
| | regular | 4.66 | μm | | | | | |
| | droplets | 4.66 | μm | | | | | |

*statistical significance compared with CR01 at p < 0.05

Formulations without Solubilizing Agents

Formulations provided in Table 11.

TABLE 11
Compositions of formulations without solubilizing agents.

| | Composition of Formulation | |
|---|---|---|
| | containing DMVT-505 (% w/w) | |
| Component | CR84 | CR85 |
| DMVT-505 | 1 | 1 |
| Propylene glycol | 10 | — |
| Transcutol P | 2 | — |
| Polysorbate 80 | 1.5 | 1.5 |
| Mineral Oil | 10 | 10 |
| Emulsifying wax. Nonionic | 7.2 | 7.2 |
| (Polawax NF) | | |
| Steareth 2, pastellated | 2.2 | 2.2 |
| Steareth 20, pastellated | 0.7 | 0.7 |
| Benzoic acid | 0.25 | 0.25 |
| Butylated hydroxytoluene (BHT) | 0.1 | 0.1 |
| Water | 64.67 | 76.67 |
| Sodium citrate dehydrate | 0.19 | 0.19 |
| Citric acid monohydrate | 0.09 | 0.09 |
| EDTA di-sodium | 0.1 | 0.1 |
| Total | 100 | 100 |
| Formulation suitable | Y | Y |

The results of the compositions of the formulations without solubilizing agents in either the oil phase (CR84; medium chain triglycerides removed) or aqueous phase (CR85; Transcutol and propylene glycol removed) is shown in Table 12.

TABLE 12
Physical Characteristics of formulations with solubilizing agents

| | | Instability |
|---|---|---|
| Formulation | Description | Index |
| CR84 | Uniform distribution of | 0.100 |
| | regularly sized droplets | |
| CR85 | Uniform distribution of | 0.190 |
| | regularly sized droplets | |

Additional physical characteristics for the formulations described herein, such as in vitro skin permeation (IVPT), in vitro epidermis penetration, and in vitro dermis penetration, are provided in Table 13.

TABLE 13
Physical Characteristics

| | | IVPT | | | | | |
|---|---|---|---|---|---|---|---|
| | | (Receptor | Statistical | Epidermis | Statistical | Dermis | Statistical |
| | | Solution) | Different | Concentration | Different | Concentration | Different |
| Workstream | Formulation | ng/cm² | from CR01 | (ng) | from CR01 | (ng) | from CR01 |
| 3 | CR50 | 38.48 | No | 1392 | No | 2596 | No |
| 3 | CR47 | 19.33 | Yes | 1167 | No | 3460 | No |
| 3 | CR51 | 16.58 | Yes | 1662 | No | 2402 | Yes |
| CWS | CR64 | 279.00 | Yes | 2204 | Yes | 6428 | No |
| CWS | CR70 | 145.55 | Yes | 1958 | Yes | 2658 | No |
| CWS | CR58 | 20.18 | No | 4044 | Yes | 3504 | No |
| CWS | CR61 | 13.06 | No | 1524 | No | 2392 | No |
| CWS | CR55 | 11.32 | No | 1731 | Yes | 5150 | No |
| CWS | CR59 | 11.09 | No | 1299 | No | 2152 | No |
| CWS | CR45 | 9.66 | No | 1752 | Yes | 5960 | No |
| CWS | CR71 | 34.25 | No | 2696 | No | 12108 | Yes |
| CWS | CR80 | 13.37 | No | 3374 | No | 6550 | No |
| CWS | CR76 | 7.61 | Yes | 2624 | No | 1635 | Yes |

### Conclusions

The results of the pre-formulation experiments demonstrated that DMVT-505 had high solubility (>7% w/w) in fixed oils, fatty alcohols, glycols and volatile solvent. Experiments conducted on the stability of DMVT-505 in individual excipients also allowed for the identification of suitable alternative oil and aqueous phase excipients, and this information was used during the formulation development as the basis of solvent system design.

For WS3, aqueous phases were designed and DMVT-505 solubility in such systems was determined, ahead of formulation development where formulations were sequentially developed, tested and optimized using accelerated physical stability data generated primarily via the LUMiSizer platform. This allowed for the development of formulations which theoretically had a similar physical stability profile to CR01 while allowing for the substitution of aqueous (WS3) phase components for others identified during pre-formulation. Additionally, combined aspects of each workstream (WS2/3/4) were developed with optimal physical stability and to limit the partitioning of the drug between the oil and aqueous phases.

Formulation candidates were then selected and progressed to short-term (2 week) stability testing. Additionally, formulations with alternative antioxidant-systems were also included in the short-term stability experiments. Formulations were assessed for drug content/purity, visual appearance, microscopic appearance with droplet sizing, apparent pH, Brookfield viscosity and LUMiSizer (at 40° C. and at t=0 only). Most formulations remained chemically stable over the 2-week period (purity>99%). However, as a color change was observed in active formulations from WS3 and WS2/3/4 workstream formulations containing PEG 400 and Transcutol P (e.g., CR47) and not corresponding placebo formulation, there may be slight drug degradation which cannot be observed using the current analytical method. Out of the formulations tested, the majority remained physically stable.

A range of alternative DMVT-505 formulations were developed for use in in vitro permeation and penetration experiments. The data generated during this study demonstrated that alternative oil phases, and aqueous phases and emulsifier systems may be employed in different combinations to result in formulations which are similar to CR01 in terms of physical and chemical stability without a noted difference in formulation physical stability or DMVT-505 chemical stability. However, it was shown that employing alternative emulsifier systems without changing either the original oil or aqueous phase had an impact on formulation stability, when compared to CR01.

Formulations were evaluated using IVPT with ex vivo human skin mounted on flow through diffusion cells (MedFlux-HT®). A total of four donors were used to test all formulations, and CR01 was applied on each donor as a bridge formulation. The intent of this testing was to compare the permeation and penetration of the test formulations to CR01, which all contained 1% w/w DMVT-505. Receptor solution (citrate phosphate buffer (pH 4.0)+0.01% Brij O20 w/v+0.1% sodium azide+20 mM ascorbic acid) and extraction fluid (90:10 v/v acetonitrile:water) developed during 386-1807F-03 were employed in this study. Receptor solution was collected every 2 hours for 36 hours and tissue was harvested at the 36-hour timepoint.

A summary table that lists the formulations tested with a designation of whether or not the receptor solution values and/or skin levels were determined to be statistically different from CR01 is included within the main body of the study report.

Overall, the formulations CR64, CR70, and Benvitimod delivered higher (p<0.05) amounts of DMVT-505 to the receptor solution compared to CR01, and formulation CR76 delivered lower (p<0.05) amounts of DMVT-505 to the receptor solution compared to CR01.

Additionally, the formulations CR58, Benvitimod cream, CR64, CR70, CR45, and CR55 delivered greater (p<0.05) amounts of DMVT-505 to the epidermis compared to CR01. CR76 delivered lower (p<0.05) amounts of DMVT-505 to the dermis compared to CR01, while CR71 delivered higher (p<0.05) amounts of DMVT-505 to the dermis compared to CR01.

### Example 9: Development of Additional Formulations

The Sponsor developed a cream formulation (CR01) containing tapinarof (DMVT-505) as a topical product for the potential treatment of psoriasis and atopic dermatitis. Additional formulations were manufactured with differing oil (workstream 2 (WS2)) phases, and different emulsifiers (WS4) from CR01 for in vitro permeation and penetration test (IVPT) evaluation.

Pre-formulation experiments and the development of formulations (including short-term stability) for use in performance testing to achieve the above aim were performed.

Pre-formulation experiments were performed to understand the solubility and stability of the drug in excipients. In relation to drug/excipient compatibility, DMVT-505 was stable for 2 weeks (peak purity>98%) in most excipients at 40° C., with the exception of MCTs and DMI, which may be due to oxidation. Based on the pre-formulation data, oil-based solvent systems were designed for WS2. Formulations based on these solvent systems, along with formulations with alternative emulsifiers (WS4) were developed. Further formulations were prepared combining suitable phases developed during all the workstreams to obtain formulations to limit the suspected drug partitioning between the aqueous and oil phases (WS2/3/4).

Formulation candidates, pooled from all workstreams, were then selected for short-term (2 week) stability testing. Formulations were assessed for drug content/purity, visual appearance, microscopic appearance with droplet sizing, apparent pH, Brookfield viscosity and LUMiSizer (at 40° C. at t=0 only). Drug in the majority of formulations appeared chemically stable over the 2-week period (purity>99%), except a small number of the formulations containing alternative antioxidants (i.e., without EDTA and another formulation with ascorbic acid) for which a slight drop in drug purity was observed. However, as a color change was observed in active formulations from WS2/3/4 formulations containing PEG 400 and Transcutol P and not corresponding placebo formulation, there may be slight drug degradation which cannot be detected on the current analytical method.

Formulations were evaluated using IVPT with ex vivo human skin mounted on flow through diffusion cells (MedFlux-HT®). A total of four donors were used to test all formulations, and CR01 was applied on each donor as a bridge formulation. The intent of this testing was to compare the permeation and penetration of the test formulations to CR01, which all contained 1% w/w DMVT-505. Receptor solution (citrate phosphate buffer (pH 4.0)+0.01% Brij O20 w/v+0.1% sodium azide+20 mM ascorbic acid) and extraction fluid (90:10 v/v acetonitrile:water) developed previously. Receptor solution was collected every 2 hours for 36 hours and tissue was harvested at the 36-hour timepoint.

Summary tables listing the results of the formulations tested regarding physical characteristics and skin permeation or penetration are provided below.

Formulations with Alternative Oil Phases (WS2)

Formulations are provided in Tables 14A-14D.

CR28, 30, 32, 34, 35—The formulation showed no change in the transmission over time. Consequently, these samples exhibited minimal to no phase separation/creaming behaviour and are therefore more likely to be physically stable during storage.

CR27 and 29 showed slight creaming behaviour, as observed by the increase in transmission at the bottom of the cell over time as phases separate. Notably, CR01 did not exhibit such behaviour, as evidenced by the low instability index observed and the transmission profile.

CR26, 31 and 33 show significant creaming behaviour. Throughout the testing, the instability indices shown in Table 15 correlate well with the visual analysis summarized above.

The microscopic observations broadly corroborate the above transmission data, where large droplet size and heterogenous distributions were evident in those formulations in which physical instability was observed via the LUMiSizer.

Accelerated physical stability testing via centrifugation was in agreement with the LUMiSizer data.

TABLE 14A
Composition of formulations with alternative oil phases (WS2)

| | | CR26 | CR27 | CR27 |
|---|---|---|---|---|
| Excipients and API | Original | PBO | PBO | ACT |
| Solvent system | (CR01) | SSO04 | SSO05 | SSO05 |
| DMVT-505 | — | — | — | 1.00 |
| Propylene glycol | 10.00 | 10.00 | 10.00 | 10.00 |
| Transcutol P | 2.00 | 2.00 | 2.00 | 2.00 |
| Emulsifying wax. | 7.20 | 7.20 | 7.20 | 7.20 |
| Nonionic (Polawax | | | | |
| NF) | | | | |
| Medium chain | 10.00 | 8.00 | 6.00 | 6.00 |
| triglycerides | | | | |
| (Crodamol GTCC) | | | | |
| Castor oil | — | — | — | — |
| IPM (isopropyl | — | 2.00 | — | — |
| myristate) | | | | |
| IPP (isopropyl | — | — | 1.00 | 1.00 |
| palmitate) | | | | |
| Crodamol ISIS | — | — | 3.00 | 3.00 |
| Kollicream OD | — | — | — | — |
| (Octyldodecanol) | | | | |
| Kollicream OA (Oleyl | — | — | — | — |
| alcohol) | | | | |
| Kollicream 3C | — | — | — | — |
| (Cocoyl | | | | |
| Caprylocaprate) | | | | |
| Crodamol IPIS | — | — | — | — |
| Polysorbate 80 (Tween | 1.50 | 1.50 | 1.50 | 1.50 |
| 80) | | | | |
| Steareth 2 (Brij S2) | 1.80 | 1.75 | 1.80 | 1.80 |
| Steareth 20 (Brij S20) | 1.10 | 1.15 | 1.10 | 1.10 |
| Benzoic acid | 0.25 | 0.25 | 0.25 | 0.25 |
| Butylated | 0.10 | 0.10 | 0.10 | 0.10 |
| hydroxytoluene (BHT) | | | | |
| Water | 65.67 | 65.67 | 65.67 | 65.67 |
| Sodium citrate | 0.19 | 0.19 | 0.19 | 0.19 |
| dihydrate | | | | |
| Citric acid | 0.09 | 0.09 | 0.09 | 0.09 |
| monohydrate | | | | |
| EDTA- di-sodium | 0.10 | 0.10 | 0.10 | 0.10 |
| Total | 100.00 | 100.00 | 100.00 | 100.00 |
| Required HLB | 11.00 | 11.10 | 10.90 | 10.90 |
| Formulation suitable | N/A | N- | Y | (Y) |
| | | physically | | |
| | | instable | | |

(Y)—potentially suitable,
Y—suitable

TABLE 14B
Composition of formulations with alternative oil phases (WS2)

| | Composition (% w/w) of creams with alternative oil phases | | | | | | |
|---|---|---|---|---|---|---|---|
| Composition (% w/w) of creams | | CR28 | CR28 | CR29 | CR29 | CR30 | CR30 |
| with alternative oil phases | Original | PBO | ACT | PBO | ACT | PBO | ACT |
| Solvent system | (CR01) | SSO06 | SSO06 | SSO07 | SSO07 | SSO08 | SSO08 |
| DMVT-505 | — | — | 1 | — | 1 | — | 1 |
| Propylene glycol | 10 | 10 | 10 | 10 | 10 | 10 | 10 |
| Transcutol P | 2 | 2 | 2 | 2 | 2 | 2 | 2 |
| Emulsifying wax. Nonionic | 7.2 | 7.2 | 7.2 | 7.2 | 7.2 | 7.2 | 7.2 |
| (Polawax NF) | | | | | | | |
| Medium chain triglycerides | 10 | — | — | 6 | 6 | — | — |
| (Crodamol GTCC) | | | | | | | |
| IPM (isopropyl myristate) | — | — | — | 2 | 2 | — | — |
| Jojoba oil | — | 5 | 5 | — | — | — | — |
| Safflower oil | — | — | — | 2 | 2 | — | — |
| Olive oil | — | 5 | 5 | — | — | — | — |
| IPP (isopropyl palmitate) | — | — | — | — | — | 5 | 5 |
| Mineral oil | — | — | — | — | — | 5 | 5 |
| Polysorbate 80 (Tween 80) | 1.5 | — | — | 1.5 | 1.5 | 1.5 | 1.5 |
| Steareth 2 (Brij S2) | 1.8 | 3.7 | 3.7 | 2 | 2 | 2.2 | 2.2 |
| Steareth 20 (Brij S20) | 1.1 | 0.7 | 0.7 | 0.9 | 1.1 | 0.7 | 0.7 |
| Benzoic acid | 0.25 | 0.25 | 0.25 | 0.25 | 0.25 | 0.25 | 0.25 |
| Butylated hydroxytoluene | 0.1 | 0.1 | 0.1 | 0.1 | 0.1 | 0.1 | 0.1 |
| (BHT) | | | | | | | |
| Water | 65.67 | 65.67 | 64.67 | 65.67 | 64.67 | 65.67 | 64.67 |
| Sodium citrate dihydrate | 0.19 | 0.19 | 0.19 | 0.19 | 0.19 | 0.19 | 0.19 |
| Citric acid monohydrate | 0.09 | 0.09 | 0.09 | 0.09 | 0.09 | 0.09 | 0.09 |
| EDTA- di-sodium | 0.1 | 0.1 | 0.1 | 0.1 | 0.1 | 0.1 | 0.1 |
| Total | 100 | 100 | 100 | 100 | 100 | 10 | 10 |
| Required HLB | 11 | 6.5 | 6.5 | 10.5 | 10.5 | 10 | 10 |
| Formulation suitable | N/A | Y | (Y) | Y | (Y) | Y | Y |

(Y) - potentially suitable, Y - suitable

TABLE 14C
Composition of formulations with alternative oil phases (WS2)

| | | CR31 | CR32 | CR32 | CR33 |
|---|---|---|---|---|---|
| Excipients and API | Original | PBO | PBO | ACT | ACT |
| Solvent system | (CR01) | SSO09 | SSO10 | SSO10 | SSO11 |
| DMVT-505 | — | — | — | 1.00 | 1.00 |
| Propylene glycol | 10.00 | 10.00 | 10.00 | 10.00 | 10.00 |
| Transcutol P | 2.00 | 2.00 | 2.00 | 2.00 | 2.00 |
| Emulsifying wax. Nonionic | 7.20 | 7.20 | 7.20 | 7.20 | 7.20 |
| (Polawax NF) | | | | | |
| Medium chain triglycerides | — | — | — | — | 8.00 |
| (Miglyol 810) | | | | | |
| Medium chain triglycerides | 10.00 | — | — | — | — |
| (Crodamol GTCC) | | | | | |
| Jojoba oil | — | — | 5.00 | 5.00 | — |
| Sesame oil | — | 5.00 | — | — | — |
| Almond oil | — | — | 5.00 | 5.00 | — |
| Soyabean oil | — | 5.00 | — | — | — |
| Mineral oil | — | — | — | — | 2.00 |
| Polysorbate 80 (Tween 80) | 1.50 | — | — | — | 1.50 |
| Steareth 2 (Brij S2) | 1.80 | 3.50 | 3.90 | 3.90 | 1.85 |
| Steareth 20 (Brij S20) | 1.10 | 0.90 | 0.50 | 0.50 | 1.05 |
| Benzoic acid | 0.25 | 0.25 | 0.25 | 0.25 | 0.25 |
| Butylated hydroxytoluene (BHT) | 0.10 | 0.10 | 0.10 | 0.10 | 0.10 |
| Water | 65.67 | 65.67 | 65.67 | 64.67 | 64.67 |
| Sodium citrate dihydrate | 0.19 | 0.19 | 0.19 | 0.19 | 0.19 |
| Citric acid monohydrate | 0.09 | 0.09 | 0.09 | 0.09 | 0.09 |
| EDTA- di-sodium | 0.10 | 0.10 | 0.10 | 0.10 | 0.10 |
| Total | 100.00 | 100.00 | 100.00 | 100.00 | 100.00 |
| Required HLB | 11.00 | 7.00 | 6.00 | 6.00 | 10.80 |
| Formulation suitable | N/A | N | Y | Y | (Y) |

(Y)—potentially suitable,
Y—suitable

TABLE 14D
Composition of formulations with alternative oil phases (WS2)

| | Composition (% w/w) of creams | | | | | | |
|---|---|---|---|---|---|---|---|
| | with alternative oil phases | | | | | | |
| | | CR34 | CR34 | CR35 | CR35 | | |
| Excipients and API | Original | PBO | ACT | PBO | ACT | CR36 | CR38 |
| Solvent system | (CR01) | SSO13 | SSO13 | SSO12 | SSO12 | SSO12 | SSO12 |
| DMVT-505 | — | — | 1 | — | 1 | 1 | 1 |
| Propylene glycol | 10 | 10 | 10 | 10 | 10 | 10 | 10 |
| Transcutol P | 2 | 2 | 2 | 2 | 2 | 2 | 2 |
| Emulsifying wax. Nonionic | 7.2 | 7.2 | 7.2 | 7.2 | 7.2 | 7.2 | 7.2 |
| (Polawax NF) | | | | | | | |
| Medium chain triglycerides | — | — | — | — | — | — | — |
| (Miglyol 810) | | | | | | | |
| Medium chain triglycerides | 10 | 2 | 2 | 5 | 5 | — | — |
| (Crodamol GTCC) | | | | | | | |
| Mineral oil | — | 8 | 8 | 5 | 5 | — | — |
| Jajoba oil | — | — | — | — | — | 5 | — |
| Olive oil | — | — | — | — | — | 5 | 10 |
| Polysorbate 80 (Tween 80) | 1.5 | 1.5 | 1.5 | 1.5 | 1.5 | 1.5 | 0.4 |
| Isopropyl palmitate | — | — | — | — | — | — | — |
| Steareth 2 (Brij S2) | 1.8 | 2.1 | 2.1 | 2 | 2 | 1.8 | 3.5 |
| Steareth 20 (Brij S20) | 1.1 | 0.8 | 0.8 | 0.9 | 0.9 | 1.1 | 0.5 |
| Benzoic acid | 0.25 | 0.25 | 0.25 | 0.25 | 0.25 | 0.25 | 0.25 |
| Butylated hydroxytoluene | 0.1 | 0.1 | 0.1 | 0.1 | 0.1 | 0.1 | 0.1 |
| (BHT) | | | | | | | |
| Water | 65.67 | 65.67 | 64.67 | 65.67 | 65.67 | 65.67 | 65.67 |
| Sodium citrate dihydrate | 0.19 | 0.19 | 0.19 | 0.19 | 0.19 | 0.19 | 0.19 |
| Citric acid monohydrate | 0.09 | 0.09 | 0.09 | 0.09 | 0.09 | 0.09 | 0.09 |
| EDTA- di-sodium | 0.1 | 0.1 | 0.1 | 0.1 | 0.1 | 0.1 | 0.1 |
| Total | 100 | 100 | 100 | 100 | 100 | 100 | 100 |
| Required HLB | 11 | 10.2 | 10.2 | 10.5 | 10.5 | 6.5 | 7 |
| Formulation suitable | N/A | Y | Y | Y | Y | Y | Y |

TABLE 15
Physical Characteristics of formulations with alternative oil phases

| | | | | | | Permeation- | | |
|---|---|---|---|---|---|---|---|---|
| | | | | | | Mean | | |
| | | | | | | Cumulative | | |
| | | Droplet | | | Mean | Amount | | |
| | | sizing | | | average | DMVT-505 | Penetration | Penetration |
| | | (diameter, | | Instability | viscosity | delivered to | in Epidermis | in Dermis |
| Formulation | Description | n = 6) | | Index | (mPa · s) | receptor(ng/cm²) | (ng)-Mean | (ng)-Mean |
| CR01 ACT | Uniform | 3.54 | μm | 0.061 | | | | |
| MP | distribution | 3.81 | μm | | | | | |
| | of regular | 3.74 | μm | | | | | |
| | sized droplets | 3.25 | μm | | | | | |
| | | 3.11 | μm | | | | | |
| | | 3.39 | μm | | | | | |
| CR01 ACT | Uniform | 0.60 | μm | 0.085 | 148,667 | 25.15, 39.65, | 997, 1338, | 2976, 5112, |
| Sponsor | distribution | 0.78 | μm | | | 21.23, 23.94 | 534, 4814 | 3836, 4308 |
| | of regular | 0.44 | μm | | | depending | depending | depending |
| | sized droplets | 0.71 | μm | | | on which | on which | on which |
| | | 0.44 | μm | | | trial and | trial and | trial and |
| | | 0.70 | μm | | | skin donor | skin donor | skin donor |
| CR01 PBO | Uniform | 7.85 | μm | 0.018 | | | | |
| | distribution | 7.08 | μm | | | | | |
| | of disperse | 8.44 | μm | | | | | |
| | phase, mostly | 6.99 | μm | | | | | |
| | regular | 7.08 | μm | | | | | |
| | droplets | 6.94 | μm | | | | | |
| CR26 PBO | Non-uniform | 13.11 | μm | 0.192 | | | | |
| | distribution | 13.97 | μm | | | | | |
| | of disperse | 9.55 | μm | | | | | |
| | phase, | 10.29 | μm | | | | | |
| | irregular | 8.80 | μm | | | | | |
| | droplets. | 10.05 | μm | | | | | |
| CR27 ACT | Uniform | 10.42 | μm | 0.070 | 126,250 | 33.05 | 1694 | 7320 |
| | distribution | 9.46 | μm | | | | | |
| | of disperse | 13.19 | μm | | | | | |
| | phase, | 14.67 | μm | | | | | |
| | irregular | 11.56 | μm | | | | | |
| | droplets. | 12.42 | μm | | | | | |
| CR27 PBO | Uniform | 9.17 | μm | 0.040 | 134,083 | | | |
| | distribution | 9.67 | μm | | | | | |
| | of disperse | 9.74 | μm | | | | | |
| | phase, | 10.20 | μm | | | | | |
| | irregular | 8.63 | μm | | | | | |
| | droplets. | 7.81 | μm | | | | | |
| CR28 ACT | Non-uniform | 6.25 | μm | 0.078 | 104,790 | 120.19* | 963 | 1646 |
| | distribution | 7.26 | μm | | | | | |
| | of disperse | 7.79 | μm | | | | | |
| | phase, | 7.08 | μm | | | | | |
| | irregular | 11.26 | μm | | | | | |
| | droplets | 7.75 | μm | | | | | |
| CR28 PBO | Non-uniform | 23.45 | μm | 0.009 | 257,250 | | | |
| | distribution | 20.40 | μm | | | | | |
| | of disperse | 16.69 | μm | | | | | |
| | phase, | 15.20 | μm | | | | | |
| | irregular | 16.26 | μm | | | | | |
| | droplets. | 17.26 | μm | | | | | |
| CR29 ACT | Uniform | 10.43 | μm | 0.055 | 292,583 | 36.99 | 1170 | 1822 |
| | distribution | 15.09 | μm | | | | | |
| | of disperse | 10.89 | μm | | | | | |
| | phase, | 8.95 | μm | | | | | |
| | irregular | 9.35 | μm | | | | | |
| | droplets. | 8.29 | μm | | | | | |
| CR29 PBO | Uniform | 10.02 | μm | 0.050 | 219,500 | | | |
| | distribution | 11.58 | μm | | | | | |
| | of disperse | 8.63 | μm | | | | | |
| | phase, mostly | 7.62 | μm | | | | | |
| | regular | 10.81 | μm | | | | | |
| | droplets. | 7.66 | μm | | | | | |
| CR30 ACT | Uniform | 3.18 | μm | 0.014 | 157,417 | 31.67 | 892 | 4204 |
| | distribution | 2.89 | μm | | | | | |
| | of disperse | 6.07 | μm | | | | | |
| | phase, mostly | 4.00 | μm | | | | | |
| | regular | 3.63 | μm | | | | | |
| | droplets | 3.69 | μm | | | | | |
| CR30 PBO | Uniform | 9.50 | μm | 0.013 | 293,417 | | | |
| | distribution | 8.20 | μm | | | | | |
| | of disperse | 7.54 | μm | | | | | |
| | phase, mostly | 8.97 | μm | | | | | |
| | regular | 8.76 | μm | | | | | |
| | droplets. | 7.43 | μm | | | | | |
| CR31 PBO | Non-uniform | 18.77 | μm | 0.095 | | | | |
| | distribution | 14.95 | μm | | | | | |
| | of disperse | 16.23 | μm | | | | | |
| | phase, | 11.81 | μm | | | | | |
| | irregular | 11.74 | μm | | | | | |
| | droplets. | 9.37 | μm | | | | | |
| CR32 ACT | Non-uniform | 11.25 | μm | 0.019 | 189,250 | 73.78* | 776 | 6626* |
| | distribution | 12.34 | μm | | | | | |
| | of disperse | 9.76 | μm | | | | | |
| | phase, | 15.71 | μm | | | | | |
| | irregular | 10.00 | μm | | | | | |
| | droplets | 9.41 | μm | | | | | |
| CR32 PBO | Non-uniform | 17.36 | μm | 0.008 | 159,583 | | | |
| | distribution | 16.36 | μm | | | | | |
| | of disperse | 14.72 | μm | | | | | |
| | phase, | 16.63 | μm | | | | | |
| | irregular | 12.69 | μm | | | | | |
| | droplets. | 13.65 | μm | | | | | |
| CR33 ACT | Non-uniform | 28.48 | μm | 0.061 | 104,780 | 25.48 | 1063 | 4246 |
| | distribution | 23.06 | μm | | | | | |
| | of disperse | 19.91 | μm | | | | | |
| | phase, | 16.72 | μm | | | | | |
| | irregular | 32.57 | μm | | | | | |
| | droplets | 35.12 | μm | | | | | |
| CR34 ACT | Non-uniform | 7.02 | μm | 0.013 | 220,250 | 26.17 | 927 | 2962 |
| | distribution | 8.25 | μm | | | | | |
| | of disperse | 8.63 | μm | | | | | |
| | phase, | 8.79 | μm | | | | | |
| | irregular | 8.37 | μm | | | | | |
| | droplets | 6.85 | μm | | | | | |
| CR34 PBO | Uniform | 8.11 | μm | 0.012 | 187,667 | | | |
| | distribution | 7.71 | μm | | | | | |
| | of disperse | 7.60 | μm | | | | | |
| | phase, mostly | 6.38 | μm | | | | | |
| | regular | 6.03 | μm | | | | | |
| | droplets. | 8.80 | μm | | | | | |
| CR35 ACT | Uniform | 8.45 | μm | 0.062 | 140,333 | 28.02 | 631 | 3686 |
| | distribution | 9.00 | μm | | | | | |
| | of disperse | 10.59 | μm | | | | | |
| | phase, | 7.98 | μm | | | | | |
| | irregular | 9.62 | μm | | | | | |
| | droplets | 8.55 | μm | | | | | |
| CR35 PBO | Non-uniform | 9.53 | μm | 0.012 | 91,426 | | | |
| | distribution | 7.86 | μm | | | | | |
| | of disperse | 8.29 | μm | | | | | |
| | phase, mostly | 7.42 | μm | | | | | |
| | regular | 5.70 | μm | | | | | |
| | droplets. | 7.10 | μm | | | | | |
| CR36 ACT | Non-uniform | 9.57 | μm | 0.073 | 58,749 | 52.59* | 923 | 3552 |
| | distribution | 8.18 | μm | | | | | |
| | of disperse | 12.12 | μm | | | | | |
| | phase, | 8.97 | μm | | | | | |
| | irregular | 10.53 | μm | | | | | |
| | droplets | 8.80 | μm | | | | | |
| CR38 ACT | Non-uniform | 25.45 | μm | 0.046 | 267,917 | 107.68* | 1197 | 4292 |
| | distribution | 11.01 | μm | | | | | |
| | of disperse | 13.18 | μm | | | | | |
| | phase, | 10.89 | μm | | | | | |
| | irregular | 11.56 | μm | | | | | |
| | droplets | 10.49 | μm | | | | | |
| CR39 ACT | Uniform | 5.86 | μm | 0.227 | | | | |
| | distribution | 6.44 | μm | | | | | |
| | of disperse | 6.03 | μm | | | | | |
| | phase, mostly | 7.45 | μm | | | | | |
| | regular | 5.48 | μm | | | | | |
| | droplets | 9.19 | μm | | | | | |

*statistical significance compared with CR01 at p < 0.05

### Development of Alternative Emulsifiers (WS4)

Formulations are provided in Tables 16A-16C.

A brief summary of the approach is presented below:

CR13, and CR14 were formulated replacing the emulsifying wax for cetostearyl alcohol, cetyl alcohol stearic acid and cetyl palmitate, respectively. All formulations showed greater stability under accelerated conditions than CR01.

CR17 was developed replacing polysorbate 80 for polysorbate 60 and steareth 20 for steareth 21. The resulting formulation exhibited good accelerated stability.

CR21 replaced the emulsifier system for Cithrol™ GMS40-Cetomacrogol 1000. CR21 showed optimal stability.

CR65, CR72 and CR73 were developed replacing the surfactant system with Tefose 63 with co-emulsifier Labrafil® M 1944. CR72 included HEC to increase viscosity and in turn potentially increase physical stability. Finally, CR73 explored higher levels of the emulsifiers, in an attempt to increase physical stability of the formulation.

Physical characteristics for each formulation are provided in Table 17.

TABLE 16A
Composition (% w/w) of formulations with alternative emulsifiers (WS4)

| | CR01 | CR13 | CR13 | CR14 | CR14 |
|---|---|---|---|---|---|
| Excipient | (Original) | PBO | ACT | PBO | ACT |
| DMVT-505 | — | — | 1.00 | — | 1.00 |
| Propylene glycol | 10.00 | 10.00 | 10.00 | 10.00 | 10.00 |
| Transcutol P | 2.00 | 2.00 | 2.00 | 2.00 | 2.00 |
| Medium chain | 10.00 | 10.00 | 10.00 | 10.00 | 10.00 |
| triglycerides (Miglyol | | | | | |
| 810) | | | | | |
| Non-ionic emulsifying | 7.20 | — | — | — | — |
| wax (Polawax NF) | | | | | |
| Cetostearyl alcohol | — | 7.20 | 7.20 | — | — |
| (Crodacol ™CS50) | | | | | |
| Cetyl alcohol | — | — | — | 7.20 | 7.20 |
| Stearic acid | — | — | — | — | — |
| Cetyl palmitate | — | — | — | — | — |
| Polysorbate 80 (Tween | 1.50 | 1.50 | 1.50 | 1.50 | 1.50 |
| 80) | | | | | |
| Steareth 2 (Brij S2) | 1.80 | 1.00 | 1.00 | 1.00 | 1.00 |
| Steareth 20 (Brij S20) | 1.10 | 1.90 | 1.90 | 1.90 | 1.90 |
| Benzoic acid | 0.25 | 0.25 | 0.25 | 0.25 | 0.25 |
| Butylated | 0.10 | 0.10 | 0.10 | 0.10 | 0.10 |
| hydroxytoluene (BHT) | | | | | |
| Water | 65.67 | 65.67 | 64.67 | 65.67 | 65.67 |
| Sodium citrate dihydrate | 0.19 | 0.19 | 0.19 | 0.19 | 0.19 |
| Citric acid monohydrate | 0.09 | 0.09 | 0.09 | 0.09 | 0.09 |
| EDTA di-sodium | 0.10 | 0.10 | 0.10 | 0.10 | 0.10 |
| Total | 100.00 | 100.00 | 100.00 | 100.00 | 100.00 |
| Formulation suitable | N/A | Y | Y | Y | Y |

TABLE 16B
Composition (% w/w) of formulations
with alternative emulsifiers (WS4)

| | CR01 | CR17 | CR17 |
|---|---|---|---|
| Excipients | (Original) | PBO | ACT |
| DMVT-505 | — | — | 1.00 |
| Propylene glycol | 10.00 | 10.00 | 10.00 |
| Transcutol P | 2.00 | 2.00 | 2.00 |
| Medium chain | 10.00 | 10.00 | 10.00 |
| triglycerides (Miglyol | | | |
| 810) | | | |
| Non-ionic emulsifying | 7.20 | 7.20 | 7.20 |
| wax Polawax NF | | | |
| Polysorbate 80 | 1.50 | — | — |
| (Tween 80) | | | |
| Polysorbate 60 | — | 1.50 | 1.50 |
| (Tween 60) | | | |
| Steareth 2 (Brij S2) | 1.80 | 1.80 | 1.80 |
| Steareth 20 (Brij S20) | 1.10 | — | — |
| Steareth 21 (Brij | — | 1.10 | 1.10 |
| S721) | | | |
| Sorbitan stearate/ | — | — | — |
| Sorbitan Laureate | | | |
| (Arlacel LC) | | | |
| Glycerol monostearate | — | — | — |
| (Cithrol ™ GMS 40) | | | |
| PEG 40 stearate | — | — | — |
| (Myrj ™ S40) | | | |
| Sorbitan monostearate | — | — | — |
| (Span 60) | | | |
| Benzoic acid | 0.25 | 0.25 | 0.25 |
| Butylated | 0.10 | 0.10 | 0.10 |
| hydroxytoluene | | | |
| (BHT) | | | |
| Water | 65.67 | 65.67 | 64.67 |
| Sodium citrate | 0.19 | 0.19 | 0.19 |
| dihydrate | | | |
| Citric acid | 0.09 | 0.09 | 0.09 |
| monohydrate | | | |
| EDTA di-sodium | 0.10 | 0.10 | 0.10 |
| Total | 100.00 | 100.00 | 100.00 |
| Formulation suitable | N/A | Y | Y |

TABLE 16C
Composition (% w/w) of formulations with alternative emulsifiers (WS4)

| | CR01 | CR21 | CR21 | CR65 | CR72 | CR73 |
|---|---|---|---|---|---|---|
| Excipients | (Original) | PBO | ACT | ACT | ACT | ACT |
| DMVT-505 | — | — | 1.00 | 1.00 | 1.00 | 1.00 |
| Propylene glycol | 10.00 | 10.00 | 10.00 | 10.00 | 10.00 | 10.00 |
| Transcutol P | 2.00 | 2.00 | 2.00 | 2.00 | 2.00 | 2.00 |
| Medium chain triglycerides | 10.00 | 10.00 | 10.00 | 10.00 | 10.00 | 10.00 |
| (Miglyol 810) | | | | | | |
| Non-ionic emulsifying wax | 7.20 | 7.20 | 7.20 | 7.20 | 7.20 | 7.20 |
| Polawax NF | | | | | | |
| Cetostearyl alcohol | — | — | — | — | — | — |
| Polysorbate 80 (Tween 80) | 1.50 | — | — | — | — | — |
| Steareth 2 (Brij S2) | 1.80 | — | — | — | — | — |
| Steareth 20 (Brij S20) | 1.10 | — | — | — | — | — |
| Glycerol monostearate | — | 1.70 | 1.70 | — | — | — |
| (Cithrol ™ GMS 40) | | | | | | |
| PEG 20 Cetostearyl ether | — | 2.70 | 2.70 | — | — | — |
| (Cetomacrogol 1000) | | | | | | |
| Gelot 64 | | | | | | |
| Tefose 63 | — | — | — | 8.00 | 8.00 | 12.00 |
| Labrafil M 1944 | — | — | — | 4.00 | 4.00 | 6.00 |
| Benzoic acid | 0.25 | 0.25 | 0.25 | 0.25 | 0.25 | 0.25 |
| Butylated hydroxytoluene | 0.10 | 0.10 | 0.10 | 0.10 | 0.10 | 0.10 |
| (BHT) | | | | | | |
| Water | 65.67 | 65.67 | 64.67 | 57.07 | 56.07 | 51.07 |
| Sodium citrate dihydrate | 0.19 | 0.19 | 0.19 | 0.19 | 0.19 | 0.19 |
| Citric acid monohydrate | 0.09 | 0.09 | 0.09 | 0.09 | 0.09 | 0.09 |
| EDTA di-sodium | 0.10 | 0.10 | 0.10 | 0.10 | 0.10 | 0.10 |
| HEC (250-HHX) | — | — | — | — | 1.00 | — |
| Total | 100.00 | 100.00 | 100.00 | 100.00 | 100.00 | 100.00 |
| Formulation suitable | N/A | Y | Y | (Y) | (Y) | (Y) |

TABLE 17
Physical Characteristics of formulations with alternative emulsifiers

| | | | | | | Mean | | |
|---|---|---|---|---|---|---|---|---|
| | | | | | | Cumulative | | |
| | | Droplet | | | Mean | Amount | | |
| | | sizing | | | average | DMVT-505 | Penetration | Penetration |
| | | (diameter, | | Instability | viscosity | delivered to | in Epidermis | in Dermis |
| Formulation | Description | n = 6) | | Index | (mPa · s) | receptor(ng/cm²) | (ng)-Mean | (ng)-Mean |
| CR01 PBO | Uniform | 7.85 | μm | 0.018 | | | | |
| | distribution | 7.08 | μm | | | | | |
| | of disperse | 8.44 | μm | | | | | |
| | phase, mostly | 6.99 | μm | | | | | |
| | regular | 7.08 | μm | | | | | |
| | droplets | 6.94 | μm | | | | | |
| CR01 ACT | Uniform | 3.54 | μm | 0.061 | | | | |
| MP | distribution | 3.81 | μm | | | | | |
| | of regular | 3.74 | μm | | | | | |
| | sized droplets | 3.25 | μm | | | | | |
| | | 3.11 | μm | | | | | |
| | | 3.39 | μm | | | | | |
| CR01 ACT | Uniform | 0.60 | μm | 0.085 | 148,667 | 25.15, 39.65, | 997, 1338, | 2976, 5112, |
| Sponsor | distribution | 0.78 | μm | | | 21.23, 23.94 | 534, 4814 | 3836, 4308 |
| | of regular | 0.44 | μm | | | depending | depending | depending |
| | sized droplets | 0.71 | μm | | | on which | on which | on which |
| | | 0.44 | μm | | | trial and | trial and | trial and |
| | | 0.70 | μm | | | skin donor | skin donor | skin donor |
| CR 13 PBO | Non-uniform | 9.68 | μm | 0.028 | 170,250 | | | |
| | distribution | 10.10 | μm | | | | | |
| | of disperse | 11.57 | μm | | | | | |
| | phase, | 10.52 | μm | | | | | |
| | irregular | 11.20 | μm | | | | | |
| | droplets. | 8.80 | μm | | | | | |
| CR13 ACT | Non-uniform | 12.42 | μm | 0.013 | 158,500 | 46.40 | 3680 | 9056 |
| | distribution | 9.16 | μm | | | | | |
| | of disperse | 7.82 | μm | | | | | |
| | phase, | 9.09 | μm | | | | | |
| | irregular | 10.59 | μm | | | | | |
| | droplets. | 15.13 | μm | | | | | |
| CR14 PBO | Non-uniform | 10.03 | μm | 0.043 | 146,417 | | | |
| | distribution | 10.58 | μm | | | | | |
| | of disperse | 8.08 | μm | | | | | |
| | phase, | 8.81 | μm | | | | | |
| | irregular | 9.00 | μm | | | | | |
| | droplets. | 6.47 | μm | | | | | |
| CR14 ACT | Non-uniform | 14.66 | μm | 0.015 | 150,583 | 63.55 | 5182 | 7238 |
| | distribution | 9.95 | μm | | | | | |
| | of disperse | 10.29 | μm | | | | | |
| | phase, | 11.03 | μm | | | | | |
| | irregular | 9.20 | μm | | | | | |
| | droplets. | 9.35 | μm | | | | | |
| CR17 PBO | Uniform | 8.09 | μm | 0.013 | 7,156 | | | |
| | distribution | 7.92 | μm | | | | | |
| | of disperse | 5.76 | μm | | | | | |
| | phase, mostly | 8.22 | μm | | | | | |
| | regular | 7.45 | μm | | | | | |
| | droplets. | 6.25 | μm | | | | | |
| CR21 PBO | Non-uniform | 19.71 | μm | 0.012 | 171,667 | | | |
| | distribution | 15.24 | μm | | | | | |
| | of disperse | 13.90 | μm | | | | | |
| | phase, | 14.21 | μm | | | | | |
| | irregular | 14.08 | μm | | | | | |
| | droplets. | 11.17 | μm | | | | | |
| CR22 PBO | Non-uniform | 39.78 | μm | 0.192 | | | | |
| | distribution | 22.58 | μm | | | | | |
| | of disperse | 18.49 | μm | | | | | |
| | phase, | 21.26 | μm | | | | | |
| | irregular | 25.44 | μm | | | | | |
| | droplets | 21.82 | μm | | | | | |
| CR17 ACT | Non-uniform | 8.80 | μm | 0.007 | | 38.79 | 4452 | 4952 |
| | distribution | 11.78 | μm | | | | | |
| | of disperse | 12.09 | μm | | | | | |
| | phase, | 13.30 | μm | | | | | |
| | irregular | 14.12 | μm | | | | | |
| | droplets | 9.47 | μm | | | | | |
| CR21 ACT | Non-uniform | 10.18 | μm | 0.013 | | 41.00 | 1610 | 6906 |
| | distribution | 8.72 | μm | | | | | |
| | of disperse | 9.57 | μm | | | | | |
| | phase, | 9.35 | μm | | | | | |
| | irregular | 11.25 | μm | | | | | |
| | droplets | 8.31 | μm | | | | | |
| CR65 ACT | Uniform | 10.16 | μm | 0.042 | 258,667 | 23.61 | 1115 | 4608 |
| | distribution | 8.28 | μm | | | | | |
| | of disperse | 8.09 | μm | | | | | |
| | phase, mostly | 9.71 | μm | | | | | |
| | regular | 9.33 | μm | | | | | |
| | droplets | 9.64 | μm | | | | | |
| CR72 ACT | Non-uniform | 18.25 | μm | 0.008 | 1,311,667 | 11.13* | 1490 | 5934 |
| | distribution | 25.47 | μm | | | | | |
| | of disperse | 17.59 | μm | | | | | |
| | phase, | 18.92 | μm | | | | | |
| | irregular | 18.35 | μm | | | | | |
| | droplets | 23.86 | μm | | | | | |
| CR73 ACT | Uniform | 6.25 | μm | 0.080 | 1,420,667 | 27.38 | 3285 | 5680 |
| | distribution | 8.11 | μm | | | | | |
| | of disperse | 5.99 | μm | | | | | |
| | phase, mostly | 6.96 | μm | | | | | |
| | regular | 5.13 | μm | | | | | |
| | droplets | 7.10 | μm | | | | | |

*statistical significance compared with CR01 at p < 0.05

### Development of Formulations Combining Workstreams (WS2/3/4)

Formulations are provided in Tables 18A-18C

CR64 was developed based on the original aqueous system, combined with SSO12 aiming to increase DMVT-505 thermodynamic activity by reducing the solubility in the overall system, while maintaining the target dose. The emulsifiers from CR17 were utilized as they were amongst the most successful in obtaining a physically stable formulation in WS 4.

CR64 CR70, CR79 and CR80 are formulations containing diisopropyl adipate in the oil phase, a good solubilizer for DMVT-505, PEG 100 stearate, PEG 8 stearate and Tefose 63 were among the emulsifiers employed. Changes were made in the aqueous phase (e.g., removal of Transcutol P) and in the preservative system.

Physical characteristics for each formulation are provided in Table 19.

TABLE 18A
Composition of formulations from combined workstreams

| | CR01 | CR64 |
|---|---|---|
| Excipients and API | ACT | ACT |
| DMVT-505 | 1.00 | 1.00 |
| Propylene glycol | 10.00 | 10.00 |
| Transcutol P | 2.00 | 2.00 |
| PEG 400 | — | — |
| Ethanol | — | — |
| Arlasolve DMI (dimethyl isosorbide) | — | — |
| Emulsifying wax. Nonionic | 7.20 | — |
| (Polawax NF) | | |
| Cetostearyl alcohol | — | — |
| Cetyl alcohol | — | 8.00 |
| Medium chain triglycerides (Miglyol | 10.00 | 5.00 |
| 810) | | |
| Mineral oil | — | 5.00 |
| Polysorbate 60 (Tween 60) | — | — |
| Polysorbate 80 (Tween80) | 1.50 | — |
| Steareth 2 (Brij S2) | 1.80 | — |
| Steareth 20 (Brij S20) | 1.10 | — |
| Steareth 21 (Brij S721) | — | — |
| Arlacel 165 | — | — |
| Tefose 63 | — | — |
| Labrafil M 1944 | — | — |
| Gelot 64 | — | 4.00 |
| Benzoic acid | 0.25 | 0.250 |
| Butylated hydroxytoluene (BHT) | 0.10 | 0.10 |
| Water | 64.67 | 64.27 |
| Sodium citrate dihydrate | 0.19 | 0.19 |
| Citric acid monohydrate | 0.09 | 0.09 |
| EDTA- di-sodium | 0.10 | 0.10 |
| Total | 100.00 | 100.00 |
| WS | N/A | 2-4 |
| Formulation suitable | N/A | (Y) |

TABLE 18B
Composition of formulations from combined workstreams

| | CR01 | CR70 | CR71 |
|---|---|---|---|
| Excipients and API | (Original) | ACT | ACT |
| DMVT-505 | 1.00 | 1.00 | 1.00 |
| Propylene glycol | 10.00 | 10.00 | — |
| Transcutol P | 2.00 | — | — |
| PEG 400 | — | — | — |
| Arlasolve DMI (dimethyl | — | — | — |
| isorbide) | | | |
| Emulsifying wax. Nonionic | 7.20 | — | — |
| (Polawax NF-PA-MH) | | | |
| Medium chain tryglycerides | 10.00 | — | — |
| Stearyl alcohol | — | 0.28 | — |
| Mineral oil | 3.00 | 3.00 | 3.00 |
| Arlacel 165 | — | — | — |
| DIPA | — | 10.00 | 10.00 |
| GMS-II (Kolliwax GMS-II) | — | — | — |
| Polysorbate 60 (Tween 60) | — | 1.33 | — |
| Polysorbate 80 (Tween 80) | 1.50 | — | — |
| Steareth 2 (Brij S2) | 1.80 | — | — |
| Steareth 20 (Brij S721) | 1.10 | — | — |
| PEG 100 Stearate | — | — | — |
| PEG 8 stearate | — | — | — |
| Span 60 | — | 7.89 | — |
| Tefose 63 | — | — | 20.00 |
| Labrafil M 1944 | — | — | 3.00 |
| Benzyl alcohol | — | 1.00 | 1.00 |
| Benzoic acid | 0.25 | — | — |
| Butylated hydroxytoluene | 0.10 | 0.10 | 0.10 |
| (BHT) | | | |
| HEC (HHX) | — | — | — |
| Water | 64.67 | 65.30 | 61.80 |
| EDTA- di-sodium | 0.10 | 0.10 | 0.10 |
| Sodium citrate dihydrate | 0.19 | — | — |
| Citric acid monohydrate | 0.09 | — | — |
| Total | 100.00 | 100.00 | 100.00 |
| WS | N/A | 2-3-4 | 2-3-4 |
| Formulation suitable | N/A | (Y) | Y |
| Apparent pH | N/A | 6.75 | 3.23 |

TABLE 18C
Composition of formulations from combined workstreams

| | CR01 | CR79 | CR80 |
|---|---|---|---|
| Excipients and API | (Original) | ACT | ACT |
| DMVT-505 | 1.00 | 1.00 | 1.00 |
| Propylene glycol | 10.00 | — | 10.00 |
| Transcutol P | 2.00 | — | — |
| PEG 400 | — | — | — |
| Arlasolve DMI | — | — | — |
| (dimethyl isorbide) | | | |
| Emulsifying wax. | 7.20 | — | — |
| Nonionic (Polawax | | | |
| NF-PA-MH) | | | |
| Medium chain | 10.00 | — | — |
| tryglycerides | | | |
| Stearyl alcohol | — | 4.55 | 4.55 |
| Mineral oil | — | 3.00 | 3.00 |
| Polysorbate 80 (Tween | 1.50 | — | — |
| 80) | | | |
| Steareth 2 (Brij S2) | 1.80 | — | — |
| Steareth 21 (Brij S721) | 1.10 | — | — |
| Arlacel 165 | — | — | — |
| DIPA | — | 10.00 | 10.00 |
| GMS-II (Kolliwax | — | 6.70 | 6.70− |
| GMS-II) | | | |
| PEG 100 Stearate | — | 8.75 | 8.75 |
| Benzyl alcohol | — | 1.00 | 1.00 |
| Benzoic acid | 0.25 | — | — |
| Butylated | 0.10 | 0.10 | 0.10 |
| hydroxytoluene (BHT) | | | |
| HEC (HHX) | — | — | — |
| Carbopol 980 | — | 0.20 | — |
| Water | 64.67 | 63.87 | 54.90 |
| EDTA- di-sodium | 0.10 | — | — |
| Sodium citrate | 0.19 | — | — |
| dihydrate | | | |
| Citric acid | 0.09 | — | — |
| monohydrate | | | |
| 0.1M NaOH solution | — | 0.83 | — |
| 2nd Water addition | — | — | — |
| Total | 100.00 | 100.00 | 100.00 |
| WS | N/A | 2-3-4 | 2-3-4 |
| Formulation suitable | N/A | Y | Y |

TABLE 19
Physical Characteristics of formulations from combined workstreams

| | | | | | | Mean | | |
|---|---|---|---|---|---|---|---|---|
| | | | | | | Cumulative | | |
| | | Droplet | | | Mean | Amount | | |
| | | sizing | | | average | DMVT-505 | Penetration | Penetration |
| | | (diameter, | | Instability | viscosity | delivered to | in Epidermis | in Dermis |
| Formulation | Description | n = 6) | | Index | (mPa · s) | receptor(ng/cm²) | (ng)-Mean | (ng)-Mean |
| CR01 PBO | | | | 0.018 | | | | |
| CR01 ACT | Uniform | 3.54 | μm | 0.061 | | | | |
| MP | distribution | 3.81 | μm | | | | | |
| | of regular | 3.74 | μm | | | | | |
| | sized droplets | 3.25 | μm | | | | | |
| | | 3.11 | μm | | | | | |
| | | 3.39 | μm | | | | | |
| CR01 ACT | Uniform | 0.60 | μm | 0.085 | 148,667 | 25.15, 39.65, | 997, 1338, | 2976, 5112, |
| Sponsor | distribution | 0.78 | μm | | | 21.23, 23.94 | 534, 4814 | 3836, 4308 |
| | of regular | 0.44 | μm | | | depending | depending | depending |
| | sized droplets | 0.71 | μm | | | on which | on which | on which |
| | | 0.44 | μm | | | trial and | trial and | trial and |
| | | 0.70 | μm | | | skin donor | skin donor | skin donor |
| CR64 ACT | Non-uniform | 15.76 | μm | 0.006 | 308,500 | 279.00* | 2204* | 6428 |
| | distribution | 12.01 | μm | | | | | |
| | of disperse | 15.39 | μm | | | | | |
| | phase, | 12.39 | μm | | | | | |
| | irregular | 14.43 | μm | | | | | |
| | droplets | 16.30 | μm | | | | | |
| CR70 ACT | Non-uniform | 12.73 | μm | 0.049 | 10,718 | 145.55* | 1958* | 2658 |
| | distribution | 12.67 | μm | | | | | |
| | of disperse | 14.58 | μm | | | | | |
| | phase, | 13.07 | μm | | | | | |
| | irregular | 11.42 | μm | | | | | |
| | droplets | 12.12 | μm | | | | | |
| CR71 ACT | Uniform | 9.17 | μm | 0.006 | 1,057,083 | 34.25 | 2696 | 12108* |
| | distribution | 11.35 | μm | | | | | |
| | of disperse | 10.23 | μm | | | | | |
| | phase, | 8.91 | μm | | | | | |
| | irregular | 9.06 | μm | | | | | |
| | droplets | 8.76 | μm | | | | | |
| CR79 ACT | Uniform | 5.69 | μm | 0.010 | | | | |
| | distribution | 6.04 | μm | | | | | |
| | of disperse | 6.04 | μm | | | | | |
| | phase, | 5.35 | μm | | | | | |
| | irregular | 6.90 | μm | | | | | |
| | droplets | 5.52 | μm | | | | | |
| CR80 ACT | Uniform | 4.83 | μm | 0.010 | 276,583 | 13.37 | 3374 | 6550 |
| | distribution | 5.52 | μm | | | | | |
| | of disperse | 4.83 | μm | | | | | |
| | phase, mostly | 4.48 | μm | | | | | |
| | regular | 4.66 | μm | | | | | |
| | droplets | 4.66 | μm | | | | | |

*statistical significance compared with CR01 at p < 0.05

Additional physical characteristics for the formulations described herein, such as in vitro skin permeation (IVPT), in vitro epidermis penetration, and in vitro dermis penetration, are provided in Table 20.

TABLE 20
Physical Characteristics

| | | IVPT | | | | | |
|---|---|---|---|---|---|---|---|
| | | (Receptor | Statistical | Epidermis | Statistical | Dermis | Statistical |
| | | Solution) | Different | Concentration | Different | Concentration | Different |
| Workstream | Formulation | ng/cm² | from CR01 | (ng) | from CR01 | (ng) | from CR01 |
| 2 | CR28 | 120.19 | Yes | 963 | No | 1646 | No |
| 2 | CR38 | 107.68 | Yes | 1197 | No | 4292 | No |
| 2 | CR32 | 73.78 | Yes | 776 | No | 6626 | Yes |
| 2 | CR36 | 52.59 | Yes | 923 | No | 3552 | No |
| 2 | CR29 | 36.99 | No | 1170 | No | 1822 | No |
| 2 | CR27 | 33.05 | No | 1694 | No | 7320 | No |
| 2 | CR30 | 31.67 | No | 892 | No | 4204 | No |
| 2 | CR35 | 28.02 | No | 631 | No | 3686 | No |
| 2 | CR34 | 26.17 | No | 927 | No | 2962 | No |
| 2 | CR33 | 25.48 | No | 1063 | No | 4246 | No |
| 4 | CR14 | 63.55 | No | 5182 | No | 7238 | No |
| 4 | CR13 | 46.40 | No | 3680 | No | 9056 | No |
| 4 | CR21 | 41.00 | No | 1610 | No | 6906 | No |
| 4 | CR73 | 27.38 | No | 3285 | No | 5680 | No |
| 4 | CR65 | 23.61 | No | 1115 | No | 4608 | No |
| 4 | CR72 | 11.13 | Yes | 1490 | No | 5934 | No |
| CWS | CR64 | 279.00 | Yes | 2204 | Yes | 6428 | No |
| CWS | CR70 | 145.55 | Yes | 1958 | Yes | 2658 | No |
| CWS | CR71 | 34.25 | No | 2696 | No | 12108 | Yes |
| CWS | CR80 | 13.37 | No | 3374 | No | 6550 | No |

### Conclusions

The results of the pre-formulation experiments demonstrated that DMVT-505 had high solubility (>7% w/w) in fixed oils, fatty alcohols, glycols and volatile solvent. Experiments conducted on the stability of DMVT-505 in individual excipients also allowed for the identification of suitable alternative oil and aqueous phase excipients, and this information was used during the formulation development as the basis of solvent system design.

For WS2, oil phase solvent systems were designed and DMVT-505 solubility in such systems was determined, ahead of formulation development where formulations were sequentially developed, tested and optimized using accelerated physical stability data generated primarily via the LUMiSizer platform. This allowed for the development of formulations which theoretically had a similar physical stability profile to CR01 while allowing for the substitution of oil (WS2) phase components for others identified during pre-formulation. A number of emulsifiers and surfactant systems (WS4) were screened in order to achieve a formulation which was, theoretically, as stable as CR01. To do this, WS4 formulations were rationally designed, prepared and tested in a similar fashion to the previous workstreams. Additionally, combined aspects of each workstream (WS2/3/4) were developed with optimal physical stability and to limit the partitioning of the drug between the oil and aqueous phases.

Formulation candidates were then selected and progressed to short-term (2 week) stability testing. Additionally, formulations with alternative antioxidant-systems were also included in the short-term stability experiments. Formulations were assessed for drug content/purity, visual appearance, microscopic appearance with droplet sizing, apparent pH, Brookfield viscosity and LUMiSizer (at 40° C. and at t=0 only). Most formulations remained chemically stable over the 2-week period (purity>99%). However, as a color change was observed in active formulations WS2/3/4 workstream formulations containing PEG 400 and Transcutol P (e.g., CR47) and not corresponding placebo formulation, there may be slight drug degradation which cannot be observed using the current analytical method.

A range of alternative DMVT-505 formulations were developed for use in in vitro permeation and penetration experiments. The data generated during this study demonstrated that alternative oil phases, and aqueous phases and emulsifier systems may be employed in different combinations to result in formulations which are similar to CR01 in terms of physical and chemical stability without a noted difference in formulation physical stability or DMVT-505 chemical stability. However, it was shown that employing alternative emulsifier systems without changing either the original oil or aqueous phase had an impact on formulation stability, when compared to CR01.

Formulations were evaluated using IVPT with ex vivo human skin mounted on flow through diffusion cells (MedFlux-HT®). A total of four donors were used to test all formulations, and CR01 was applied on each donor as a bridge formulation. The intent of this testing was to compare the permeation and penetration of the 35 formulations to CR01, which all contained 1% w/w DMVT-505. Receptor solution (citrate phosphate buffer (pH 4.0)+0.01% Brij O20 w/v+0.1% sodium azide+20 mM ascorbic acid) and extraction fluid (90:10 v/v acetonitrile:water) developed during 386-1807F-03 were employed in this study. Receptor solution was collected every 2 hours for 36 hours and tissue was harvested at the 36-hour timepoint.

A summary table that lists the formulations tested with a designation of whether or not the receptor solution values and/or skin levels were determined to be statistically different from CR01 is included within the main body of the study report.

Overall, the formulations CR28, CR32, CR36, CR38, CR64, CR70, and Benvitimod delivered higher (p<0.05) amounts of DMVT-505 to the receptor solution compared to CR01, and formulation CR72 delivered lower (p<0.05) amounts of DMVT-505 to the receptor solution compared to CR01.

Additionally, the formulations Benvitimod cream, CR64, and CR70 delivered greater (p<0.05) amounts of DMVT-505 to the epidermis compared to CR01. CR32 and CR71 delivered higher (p<0.05) amounts of DMVT-505 to the dermis compared to CR01.

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore, the Examples herein are to be construed as merely illustrative and not a limitation of the scope of the present invention in any way. The embodiments of the invention in which an exclusive property or privilege is claimed are defined as follows.

1.-56. (canceled)

57. A topical pharmaceutical oil-in-water emulsion composition comprising:
about 0.1% to about 5% by weight of 3,5-dihydroxy-4-isopropyl-trans-stilbene or a pharmaceutically acceptable salt thereof,
mineral oil in an amount of about 5% to about 15% by weight, based on the total weight of the composition,
a co-solvent in an amount of about 1% to about 30% by weight, based on the total weight of the composition,
a water phase in an amount of 25% to about 85% by weight, based on the total weight of the composition,
a surfactant in an amount of about 0.1% to about 20% by weight, based on the total weight of the composition, comprising a mixture of polysorbate 80, nonionic emulsifying wax (Polawax NF), Steareth 2, and Steareth 20, and
an antioxidant.

58. The topical pharmaceutical oil-in-water emulsion composition of claim 57, further comprising an additional surfactant selected from the group consisting of non-ionic surfactants, ethoxylated fatty alcohol ethers, PEG castor oils, PEG esters, propylene glycol esters, glyceryl esters and derivatives, polymeric ethers, sorbitan derivatives, fatty alcohols, emulsifying waxes, and mixtures thereof.

59. The topical pharmaceutical oil-in-water emulsion composition of claim 57, wherein the co-solvent is selected from the group consisting of propylene glycol, di ethylene glycol monoethyl ether, transcutol, PEG 400, glycerine, ethanol, isopropanol, t-butyl alcohol, amyl alcohol, benzyl alcohol, cyclohexanedimethanol, diacetone alcohol, hexyl alcohol, tetrahydrofurfuryl alcohol, diethylene glycol monoethyl ether; carboxylic acids such as acetic acid or multi carboxylic acids; dials such as 1,2-hexanediol, butylene glycol, diethylene glycol, di propylene glycol, ethyl hexanediol, ethylene glycol, hexylene glycol, pentylene glycol, propylene glycol, tetraethylene glycol, triethylene glycol, tripropylene glycol and polyethylene glycol; polyols such as butanetriol, glycerol, 1,2,6-hexanetriol, macrogol-15-hydroxystearate, PEG 40, oleyl macrogol 6 glycerides, N-methyl-2-pyrrolidone, propylene glycol monolaurate, esters such as butyl stearate, C12-15 alkyl benzoate, C12-15 alkyl lactate, caprylic/capric triglyceride, cetearyl ethylhexanoate, cetearyl isononanoate, cetyl octanoate, cetyl palmitate, coco-caprylate/caprate, cocoglycerides, decyl oleate, dibutyl adipate, dicaprylyl carbonate, diethylhexyl adipate, di-ethylhexyl succinate, diisopropyl adipate, dioctyl malate, di-PPG-2 myreth-10 adipate, di-PPG-3 myristyl ether adipate, ethyl olcate, ethylhexyl cocoate, ethylhexyl hydroxystearate, ethylhexyl palmitate, ethylhexyl pelargonate, ethylhexyl stearate, hexyl laurate, hexyldecyl laurate, hexyldecyl stearate, isocetyl stearate, isocetyl stearoyl stearate, isodecyl oleate, isopropyl myristate, isopropyl palmitate, isostearyl neopentanoate, isotridecyl isononanoate, lauryl lactate, myristyl lactate, myristyl myristate, octyldodecyl stearoyl stearate, oleyl erucate, oleyl oleate, pentaerythrityl tetracaprylate/caprate, pentaerythrityl tetraisostearate, PPG-2 myristyl ether propionate, propylene glycol dicaprylate/dicaprate, propylene glycol isostearate, propylheptyl caprylate, and stearyl octanoatedimethyl isosorbide, propylene carbonate, hydrogenated castor oil, PPG-15 stearyl ether, octyldodecanol, and combinations and mixtures thereof.

60. The topical pharmaceutical oil-in-water emulsion composition of claim 57, wherein antioxidant is in an amount of about 0.001% to about 5%, and wherein the antioxidant is selected from the group consisting of butylated hydroxytoluene (BHT), butylated hydroxyanisole (BHA), tocopherol, propyl gallate, vitamin E TPGS and tert-Butylhydroquinone (TBHQ), and mixtures thereof.

61. The topical pharmaceutical oil-in-water emulsion composition of claim 57, wherein the composition has a pH of about 4 to about 7, and wherein the composition has a viscosity of about 10,000 mPa·s to about 300,000 mPa·s.

62. The topical pharmaceutical oil-in-water emulsion composition of claim 57, wherein the one or more additional dermatologically acceptable excipients is selected from the group consisting of pH adjusting agent, a chelating agent, a preservative, a co-solvent, a penetration enhancer, a humectant, a thickening agent, a gelling agent, a viscosity building agent, a fragrance, a colorant, and combinations thereof.

63. The topical pharmaceutical oil-in-water emulsion composition of claim 57, wherein the composition has a droplet size having a D90 of about 0.1 μm to about 35 μm.

64. The topical pharmaceutical oil-in-water emulsion composition of claim 57, wherein the composition has a droplet size having a D50 of about 0.1 μm to about 35 μm.

65. The topical pharmaceutical oil-in-water emulsion composition of claim 57, wherein the composition has an in vitro skin permeation profile of about 10 ng/cm² to about 65 ng/cm².

66. The topical pharmaceutical oil-in-water emulsion composition of claim 57, wherein the composition has an in vitro penetration into the epidermis profile of about 500 ng to about 5,200 ng.

67. The topical pharmaceutical oil-in-water emulsion composition of claim 57, wherein the composition has an in vitro penetration into the dermis profile of about 1,800 ng to about 12,000 ng.

68. A method of treating a dermatological condition or disorder in a patient in need thereof, the method comprising administering to said patient the topical pharmaceutical oil-in-water emulsion composition of claim 57.

69. A topical pharmaceutical oil-in-water emulsion composition comprising:
about 0.1% to about 5% by weight of 3,5-dihydroxy-4-isopropyl-trans-stilbene or a pharmaceutically acceptable salt thereof,
an oil phase in an amount of about 5% to about 45% by weight, based on the total weight of the composition,
a water phase in an amount of about 25% to about 85% by weight, based on the total weight of the composition,
a surfactant in an amount of about 0.1% to about 20% by weight, based on the total weight of the composition,
an antioxidant, and
a co-solvent in an amount of greater than 30% by weight, based on the total weight of the composition.

70. The topical pharmaceutical oil-in-water emulsion composition of claim 69, wherein:
the surfactant is selected from the group consisting of non-ionic surfactants, ethoxylated fatty alcohol ethers, PEG castor oils, PEG esters, propylene glycol esters, glyceryl esters and derivatives, polymeric ethers, sorbitan derivatives, fatty alcohols, emulsifying waxes, and mixtures thereof; and
the oil phase is selected from the group consisting of fatty acids, esters, esters of glycerin, fatty alcohols, waxes, sterols, unsaponifiables, siloxanes, silanes, lanolin, hydrocarbons, essential oils, vegetable oils, mineral oils, animal oils, edible oils, and mixtures thereof.

71. A method of treating a dermatological condition or disorder in a patient in need thereof, the method comprising administering to said patient the topical pharmaceutical oil-in-water emulsion composition of claim 69.

72. A topical pharmaceutical oil-in-water emulsion composition comprising:
about 0.1% to about 5% by weight of 3,5-dihydroxy-4-isopropyl-trans-stilbene or a pharmaceutically acceptable salt thereof,
an oil phase in an amount of about 5% to about 35% by weight, based on the total weight of the composition and is selected from the group consisting of cocoyl caprylocaprate, isostearyl isostearate, octyldodecanol, jajoba oil, sesame oil, walnut oil, isopropyl palmitate, isopropyl myristate, safflower oil, mineral oil, olive oil, almond oil, and combinations thereof,
a water phase in an amount of about 25% to about 85% by weight, based on the total weight of the composition,
a surfactant in an amount of about 0.1% to about 20% by weight, based on the total weight of the composition, and
an antioxidant.

73. The topical pharmaceutical oil-in-water emulsion composition of claim 72, further comprising an additional oil phase selected from the group consisting of fatty acids, esters, esters of glycerin, fatty alcohols, waxes, sterols, unsaponifiables, siloxanes, silanes, lanolin, hydrocarbons, essential oils, vegetable oils, mineral oils, animal oils, edible oils, and mixtures thereof.

74. A method of treating a dermatological condition or disorder in a patient in need thereof, the method comprising administering to said patient the topical pharmaceutical oil-in-water emulsion composition of claim 72.

75. A topical pharmaceutical oil-in-water emulsion composition comprising:
about 0.1% to about 5% by weight of 3,5-dihydroxy-4-isopropyl-trans-stilbene or a pharmaceutically acceptable salt thereof,
an oil phase in an amount of about 5% to about 45% by weight, based on the total weight of the composition,
a water phase in an amount of about 25% to about 85% by weight, based on the total weight of the composition,
a surfactant in an amount of about 1% to about 20% by weight, based on the total weight of the composition and is selected from the group consisting of oleoyl polyoxyl-6 glycerides, PEG32 Stearate, PEG 75, PEG 20 cetostearyl ether, ceteareth-12, ceteareth-15, ethylene glycol palmitostearate, cetostearyl alcohol, glycerol monostearate, stearyl alcohol, cetyl alcohol, steareth-2, steareth-20, steareth-21, and combinations thereof, and
an antioxidant.

76. A method of treating a dermatological condition or disorder in a patient in need thereof, the method comprising administering to said patient the topical pharmaceutical oil-in-water emulsion composition of claim 75.
